SEQUENCE LISTING (47 sequences)

SEQ ID NO: 1 - RNA, 19 nt, Artificial Sequence
  Synthetic sequence
ucgaucucgacuucuugag

SEQ ID NO: 2 - RNA, 19 nt, Artificial Sequence
  Synthetic sequence
uauguugaaguguaaugag

SEQ ID NO: 3 - DNA, 20 bp, Artificial Sequence
  Synthetic sequence
cctgaaccttggatcacacc

SEQ ID NO: 4 - DNA, 20 bp, Artificial Sequence
  Synthetic sequence
gcaggtcctgttggaggtaa

SEQ ID NO: 5 - DNA, 19 bp, Artificial Sequence
  Synthetic sequence
tgccatcgccaaggagtag

SEQ ID NO: 6 - DNA, 20 bp, Artificial Sequence
  Synthetic sequence
tgcacagacggtcactcaaa

SEQ ID NO: 7 - DNA, 20 bp, Artificial Sequence
  Synthetic sequence
gaccgctactcaggaggaaa

SEQ ID NO: 8 - DNA, 20 bp, Artificial Sequence
  Synthetic sequence
aggtatgagggcagcagaaa

SEQ ID NO: 9 - DNA, 21 bp, Artificial Sequence
  Synthetic sequence
ggtggttatgaccgctactca

SEQ ID NO: 10 - DNA, 18 bp, Artificial Sequence
  Synthetic sequence
agccatttggaaggacga

SEQ ID NO: 11 - DNA, 22 bp, Artificial Sequence
  Synthetic sequence
gtcgatgttagctcagtgcctc

SEQ ID NO: 12 - DNA, 22 bp, Synthetic sequence
acgaagcatatcttcagttatg

SEQ ID NO: 13 - DNA, 200 bp, Artificial Sequence
  Synthetic sequence
aaagcaagacatacagaaattatgtagttatactcagatcaacaacctactgaagattcattgaagaacctggacttactcttccaaaaggctcaaagatcacacgaagcatatcttcagttatgttgaagtgtaatgagcccacataaagcctcataggtccagcacttcccttttgtaaattgtttgccattgctgca

SEQ ID NO: 14 - protein, 20 aa, Artificial Sequence
  Synthetic sequence
ITEDMLRGIFEPFGRIESIQ

SEQ ID NO: 15 - DNA, 60 bp, Artificial Sequence
  Synthetic sequence
cataactgaagatatgcttcgtgggatctttgagccttttggaagaattgaaagtatcca

SEQ ID NO: 16 - DNA, 60 bp, Artificial Sequence
  Synthetic sequence
cataactgaagatatgcttcgtgtgatctttgagccttttggaagaattgaaagtatcca

SEQ ID NO: 17 - DNA, 60 bp, Artificial Sequence
  Synthetic sequence
cataactgaagatatgcttcgttggatctttgagccttttggaagaattgaaagtatcca

SEQ ID NO: 18 - DNA, 60 bp, Artificial Sequence
  Synthetic sequence
cataactgaagatatgcttcgtgggatctttcagccttttggaagaattgaaagtatcca

SEQ ID NO: 19 - DNA, 60 bp, Artificial Sequence
  Synthetic sequence
cataactgaagatatgcttcgtgggatctttgggccttttggaagaattgaaagtatcca

SEQ ID NO: 20 - DNA, 60 bp, Artificial Sequence
  Synthetic sequence
cataactgaagatttgcttcgtgggatctttgagccttttggaagaattgaaagtatcca

SEQ ID NO: 21 - DNA, 60 bp, Artificial Sequence
  Synthetic sequence
cataactgaagatatgcttcgtgggatctttgagtcttttggaagaattgaaagtatcca

SEQ ID NO: 22 - DNA, 60 bp, Artificial Sequence
  Synthetic sequence
cataactgaagatatgcttcgtaggatctttgagccttttggaagaattgaaagtatcca

SEQ ID NO: 23 - DNA, 60 bp, Artificial Sequence
  Synthetic sequence
cataactgaagatatgcttcgtgagatctttgagccttttggaagaattgaaagtatcca

SEQ ID NO: 24 - DNA, 16 bp, Artificial Sequence
  Synthetic sequence
aaagcaagacatacag

SEQ ID NO: 25 - DNA, 51 bp, Artificial Sequence
  Synthetic sequence
aggctcaaagatcccacgaagcatatcttcagttatgttgaagtgtaatga

SEQ ID NO: 26 - protein, 16 aa, Artificial Sequence
  Synthetic sequence
GTTTGCCATTGCTGCA

SEQ ID NO: 27 - DNA, 16 bp, Artificial Sequence
  Synthetic sequence
tttcgttctgtatgtc

SEQ ID NO: 28 - DNA, 51 bp, Artificial Sequence
  Synthetic sequence
tccgagtttctagggtgcttcgtatagaagtcaatacaacttcacattact

SEQ ID NO: 29 - DNA, 16 bp, Artificial Sequence
  Synthetic sequence
caaacggtaacgacgt

SEQ ID NO: 30 - DNA, 16 bp, Artificial Sequence
  Synthetic sequence
aaagcaagacatacag

SEQ ID NO: 31 - DNA, 51 bp, Artificial Sequence
  Synthetic sequence
aggctcaaagatcacacgaagcatatcttcagttatgttgaagtgtaatga

SEQ ID NO: 32 - DNA, 16 bp, Artificial Sequence
  Synthetic sequence
gtttgccattgctgca

SEQ ID NO: 33 - protein, 14 aa, Artificial Sequence
  Synthetic sequence
TEDMLRGIFEPFGR

SEQ ID NO: 34 - DNA, 42 bp, Artificial Sequence
  Synthetic sequence
actgaagatatgcttcgtgggatctttgagccttttggaaga

SEQ ID NO: 35 - DNA, 42 bp, Artificial Sequence
  Synthetic sequence
actgaagatatgcttcgtgtgatctttgagccttttggaaga

SEQ ID NO: 36 - DNA, 42 bp, Artificial Sequence; identical to SEQ ID NO: 35
  Synthetic sequence

SEQ ID NO: 37 - DNA, 42 bp, Artificial Sequence; identical to SEQ ID NO: 35
  Synthetic sequence

SEQ ID NO: 38 - DNA, 42 bp, Artificial Sequence; identical to SEQ ID NO: 35
  Synthetic sequence

SEQ ID NO: 39 - DNA, 21 bp, Artificial Sequence
  Synthetic sequence
ggctgggggtgcagcggattg

SEQ ID NO: 40 - DNA, 21 bp, Artificial Sequence
  Synthetic sequence
ctctatcatcgccagaagatg

SEQ ID NO: 41 - DNA, 46 bp, Artificial Sequence
  Synthetic sequence
gcggccccagcccgttcttctgggtctcctgagccctcgcccgcca

SEQ ID NO: 42 - DNA, 53 bp, Artificial Sequence
  Synthetic sequence
gcggccccagccttctgggtctcctgagccctgagccctcgcctcgcccgcca

SEQ ID NO: 43 - DNA, 23 bp, Artificial Sequence
  Synthetic sequence
gcggccccagccctcgcccgcca

SEQ ID NO: 44 - DNA, 52 bp, Artificial Sequence
  Synthetic sequence
gaggcggccccagcccgttcttctgggtctcctgagccctcgcccgccattg

SEQ ID NO: 45 - DNA, 51 bp, Artificial Sequence
  Synthetic sequence
gaggcggccccagccgttcttctgggtctcctgagccctcgcccgccattg

SEQ ID NO: 46 - DNA, 28 bp, Artificial Sequence
  Synthetic sequence
ggagacaggtgctctcggttgcacaata

SEQ ID NO: 47 - DNA, 28 bp, Artificial Sequence
  Synthetic sequence
ggagacaggtgctttcggttgcacaata

(19) United States
(12) Patent Application Publication
(10) Pub. No.: US 2018/0140578 A1
(43) Pub. Date: May 24, 2018

(54) METHODS AND COMPOSITIONS FOR THE DIAGNOSIS AND SELECTIVE TREATMENT OF CANCER

(71) Applicant: Board of Regents of the University of Texas System, Austin, TX (US)

(72) Inventors: Deepak Nijhawan, Dallas, TX (US); Ting Han, Dallas, TX (US); Nicholas H. Gaskill, Houston, TX (US)

(73) Assignee: Board of Regents of the University of Texas System, Austin, TX (US)

(21) Appl. No.: 15/821,111

(22) Filed: Nov. 22, 2017

Related U.S. Application Data
(60) Provisional application No. 62/425,732, filed on Nov. 23, 2016.
(60) Provisional application No. 62/470,073, filed on Mar. 10, 2017.

(51) Int. Cl. A61K 31/404; A61K 31/381; A61K 31/498; A61P 35/00; G01N 33/574

(52) U.S. Cl. CPC A61K 31/404; A61K 31/381; G01N 33/57484; A61P 35/00; A61K 31/498

(57) ABSTRACT

In one aspect, provided herein are methods for treating cancer in a subject, comprising: (1) identifying in the subject the presence of a mutation in a splicing factor selected from the group consisting of U2AF1, SF3B1, SRSF2, and ZRSR2; and/or determining in the subject an increased amount of DCAF15 compared to a control, and (2) inhibiting an activity of RBM39 in the subject. In some embodiments, the inhibiting step can include promoting RBM39 degradation, preferably in a DCAF15-dependent manner. Compositions are also provided.

### CROSS-REFERENCE TO RELATED APPLICATIONS

This application claims priority to and the benefit of U.S. Provisional Application Nos. 62/425,732 filed Nov. 23, 2016 and 62/470,073 filed Mar. 10, 2017, the entire disclosures of which applications are incorporated herein by reference.

### SEQUENCE LISTING

The ASCII text file submitted herewith via EFS-Web, entitled “017102Sequence.txt” created on Nov. 22, 2017, having a size of 9,715 bytes, is incorporated herein by reference in its entirety.

### FIELD

The present disclosure relates in general to methods and compositions for the diagnosis and treatment of cancer, in particular using biomarkers and/or aryl sulfonamides.

### BACKGROUND

Genome-wide studies of thousands of tumors have implicated pre-mRNA splicing as a driver of cancer progression, thus prompting active efforts to discover inhibitors of pre-mRNA splicing as a new approach for cancer treatment (1). Many of the proteins important for pre-mRNA splicing, however, have no enzymatic activity and are thus challenging to inhibit via small molecules (2, 3). The discovery of the mechanism of action for the anti-tumor activity of thalidomide, lenalidomide and polamidomide (collectively termed Immunomodulatory Drugs (IMiDs)) has provided a strategy to target otherwise undruggable proteins (4-7). IMiDs bind to Cereblon (CRBN), which is the substrate receptor for the E3 ubiquitin ligase complex CUL4-DDB1-RBX1-CRBN (CUL4-CRBN) (4). Binding to CRBN not only inhibits the endogenous E3 ubiquitin ligase activity of CUL4-CRBN but also repurposes the enzyme to ubiquitinate other proteins as neo-substrates. For example, the clinical activity of IMiDs is the result of ubiquitination and degradation of two transcription factors, IKZF1 (Ikaros) and IKZF3 (Aiolos) in multiple myeloma (6, 7). IMiDs also prompt degradation of the CSNK1A1 (CK1α) protein kinase as a means of treating 5q deletion associated myelodysplatic syndrome (7).

Indisulam (also known as E7070) is an aryl sulfonamide discovered by Eisai Pharmaceuticals in a phenotypic screen for small molecules with anti-cancer activity (FIG. 1A) (8, 9). Treatment of human cancer cell lines with indisulam resulted in cell cycle arrest in the G1 phase followed by cell death (8, 9). In a comprehensive analysis of 42 cancer cell lines, indisulam was found to be toxic to a subset of lines representing multiple different lineages (10). The selectivity of indisulam was recapitulated in pre-clinical efficacy studies using tumors derived from different human cancer cell lines grafted into immune-deficient mice (10). In multiple phase I and phase II trials involving advanced stage patients with solid tumor malignancies, indisulam monotherapy resulted in a modest number of clinical responses and stable disease in 17-36% of patients (11-18). The biologic basis for indisulam sensitivity is unknown, and there are no biomarkers available capable of predicting which patients might be more likely to respond to indisulam.

Thus, a need exists for understanding the mechanism of action of indisulam, as well as identifying predictive biomarkers that can guide the use of indisulam and other aryl sulfonamides.

### SUMMARY

In one aspect, provided herein is a method for treating cancer in a subject, comprising: (1) identifying in the subject the presence of a mutation in a splicing factor selected from the group consisting of U2AF1, SF3B1, SRSF2, and ZRSR2; and/or determining in the subject an increased amount of DCAF15 compared to a control; and (2) inhibiting an activity of RBM39 in the subject.

In some embodiments, the inhibiting step can include promoting RBM39 degradation, preferably in a DCAF15-dependent manner. The promoting step can include administering an effective amount of a compound that targets RBM39, preferably an aryl sulfonamide selected from the group consisting of indisulam, tasisulam, chloroquinoxaline sulfonamide, and analogues of each of the foregoing.

Also provided herein is a method for determining whether or not a cancer patient is likely to respond to treatment, comprising determining whether the patient's cancer cells have (1) a mutation in a splicing factor selected from the group consisting of U2AF1, SF3B1, SRSF2, and ZRSR2; and/or (2) an increased amount of DCAF15 compared to a control, wherein the mutation or increased amount indicates that the patient is likely to respond to treatment with a compound that targets RBM39, preferably an aryl sulfonamide selected from the group consisting of indisulam, tasisulam, chloroquinoxaline sulfonamide, and analogues of each of the foregoing.

Another aspect relates to a method for selectively treating a patient with cancer, comprising: identifying a patient having (1) a mutation in a splicing factor selected from the group consisting of U2AF1, SF3B1, SRSF2, and ZRSR2; and/or (2) an increased amount of DCAF15 in the patient's cancer cells compared to a control; and administering to the patient a therapeutically effective amount of a compound that targets RBM39, preferably an aryl sulfonamide selected from the group consisting of indisulam, tasisulam, chloroquinoxaline sulfonamide, and analogues of each of the foregoing.

A further aspect relates to a diagnostic kit comprising one or more reagent for determining (1) a mutation in a splicing factor selected from the group consisting of U2AF1, SF3B1, SRSF2, and ZRSR2; and/or (2) a level of DCAF15 in a sample from a cancer patient, wherein the presence of the mutation and/or an increased amount of DCAF15 compared to a control indicates responsiveness to treatment with a compound that targets RBM39, preferably an aryl sulfonamide selected from the group consisting of indisulam, tasisulam, chloroquinoxaline sulfonamide, and analogues of each of the foregoing.

In various embodiments in connection with the methods or kits disclosed herein, the mutation can be a point mutation, deletion or insertion, wherein preferably the mutation is detected by sequencing. In some embodiments, the increased amount of DCAF15 is an increase in gene copy number and/or nucleic acid expression and is determined using one or more of real-time (RT)-PCR, RNA sequencing (RNA-seq), microarray analysis, serial analysis of gene expression (SAGE), MassARRAY® technique (by Agena Bioscience), immunohistochemistry and fluorescence in situ hybridization (FISH). In certain embodiments, the control is from a non-cancerous sample of the patient.

In various embodiments, the cancer can be carcinoma, lymphoma, blastoma (including medulloblastoma and retinoblastoma), sarcoma (including liposarcoma and synovial cell sarcoma), neuroendocrine tumors (including carcinoid tumors, gastrinoma, and islet cell cancer), mesothelioma, schwannoma (including acoustic neuroma), meningioma, adenocarcinoma, melanoma, and leukemia or lymphoid malignancies. More particular examples of such cancers include squamous cell cancer (e.g., epithelial squamous cell cancer), lung cancer including small-cell lung cancer (SCLC), non-small cell lung cancer (NSCLC), adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer (including metastatic breast cancer), colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, testicular cancer, esophageal cancer, tumors of the biliary tract, as well as head and neck cancer. In some embodiments, the cancer is triple-negative metastatic breast cancer, including any histologically confirmed triple-negative (ER-, PR-, HER2-) adenocarcinoma of the breast with locally recurrent or metastatic disease (where the locally recurrent disease is not amenable to resection with curative intent). In some embodiments, the cancer is leukemia or lymphoma.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1. Mutations in RBM39 confer resistance to indisulam.

(A) Chemical structure of indisulam.
(B) Indisulam dose response curves of parental and six indisulam resistant clones.
(C) Gene mutation frequency in indisulam resistant clones. Three genes (RBM39, CD44, and NNT) are mutated in three out of six clones.
(D) Domain organization of RBM39. Indisulam-resistant mutations are clustered in the RRM2 domain of RBM39.
(E) Four mutated residues are highlighted in green on the NMR structure of the RRM2 domain of RBM39 (PDB: 2jrs).
(F) Transient transfection and expression of RBM39 with any one of the eight identified mutations protect cells from indisulam toxicity. Cells were treated with 2 μM indisulam and stained by crystal violet.
(G) Indisulam dose response curves of parental HCT-116 cells and RBM39 G268V knock-in cells.
(H) Tumor xenografts of HCT-116 cells in Nod-Scid IL-2Rγ KO mice regress following intravenous dosing of indisulam. Xenografts of HCT-116 cells expressing RBM39 G268V are resistant to indisulam (n=8 per group, ***p<0.0001, unpaired T test, two-tailed, GraphPad QuickCalcs). Error bars represent standard deviations (SD).

FIG. 2. Indisulam promotes proteasomal degradation of RBM39 via a Cullin RING type E3 ubiquitin Ligase (CRL).

(A) Indisulam treatment (2 μM for 8 hr) triggers the degradation of RBM39 in parental HCT-116 cells. RBM39 mutations identified in indisulam resistant clones abrogate RBM39 degradation following indisulam exposure.
(B) RBM39-AID is degraded in the presence of TIR1 and IAA in HCT-116 cells.
(C) IAA and TIR1 dependent degradation of RBM39-AID results in cell death. Error bars represent standard errors of the mean (SEM) (n=3).
(D) Indisulam dependent degradation of RBM39 can be blocked by bortezomib, a proteasome inhibitor. Cells were pretreated with indicated concentrations of bortezomib for 2 hours, followed by 6 hours of treatment with 2 μM indisulam. The effect of bortezomib is attenuated in a bortezomib resistant cell line.
(E) Indisulam dependent degradation of RBM39 can be blocked by MLN4924, a neddylation inhibitor. Cells were pretreated with indicated concentrations of MLN4924 for 2 hours, followed by 6 hours of treatment with 2 μM indisulam. The effect of MLN4924 is abrogated in an MLN4924 resistant cell line.
(F) Total peptide counts of CRL components from mass spec analysis of RBM39-3xFLAG complex immunopurified from untreated or indisulam treated H1155 cells.

FIG. 3. CUL4-DDB1-DDA1-DCAF15 is required for RBM39 degradation and indisulam sensitivity.

(A) Expression of dominant negative N-terminal fragments of Cullins in 293T cells and their impact on RBM39 degradation.
(B) CRISPR inactivation of DCAF15, DDB1, or DDA1 blocks indisulam dependent RBM39 degradation in H1155 cells expressing RBM39-AcGFP.
(C) Indisulam dose response curves of parental and DCAF15−/− H1155 cells. Errors bars represent SEM (n=3).
(D) Indisualm dose response curves of H1155 cells lentivirally transduced with vector or DCAF15-3xFLAG cDNA. Errors bars represent SEM (n=3).
(E) RBM39-3xFLAG co-immunoprecipitates CUL4A, CUL4B, DDB1, and DDA1 from HCT-116 cells treated with 10 μM indisulam. RBM39 G268V-3xFLAG fails to co-immunoprecipitate these proteins.
(F) Co-transfection of His-ubiquitin, RBM39-3xFLAG, and DCAF15-V5 cDNAs into 293 cells and purification of His-ubiquitin conjugated proteins reveals that indisulam induces in vivo polyubiquitination of RBM39. RBM39 G268V does not undergo indisulam dependent polyubiqutination.

FIG. 4. DCAF15-RBM39 is the target of indisulam.

(A) RBM39-3xFLAG co-immunoprecipitates CUL4A, DDB1, and DDA1 from HCT-116 lysates in an indisulam dose dependent fashion. RBM39 G268V-3xFLAG fails to associate with these proteins in vitro even with 10 μM indisulam.
(B) DCAF15-3xFLAG purified from HCT-116 cells, co-purifies recombinant RBM39Δ150 in vitro in the presence of indisulam. CUL4A-3xFLAG, CUL4B-3xFLAG, and DDB1-3xFLAG were incapable of purifiying RBM39Δ150.
(C) DCAF15-3xFLAG purified from 293T cells pulls down RBM39Δ150 in an indisulam dose dependent manner. M265L, E271Q, or P272S mutations impede RBM39Δ150 interaction with DCAF15-3xFLAG.
(D) Mass spec quantification of indisulam copurifying with indicated proteins. Error bars represent SD (n=3).
(E) A model of indisulam's mechanism of action.

FIG. 5. Tasisulam and CQS target DCAF15-RBM39.

(A) Chemical structures of CQS and tasisulam.
(B) DCAF15-3xFLAG copurifies with RBM39Δ150 in CQS and tasisulam dose dependent manner. M265L, E271Q, or P272S mutations compromise RBM39Δ150 interaction with DCAF15-3xFLAG.
(C) Indisulam, CQS, and tasisulam promote degradation of RBM39-Nluc in a concentration dependent manner. Errors bars represent SEM (n=3).
(D) CQS dose responses in parental HCT-116 cells and RBM39 G268V knock-in cells. Errors bars represent SEM (n=3).
(E) Tasisulam dose responses in parental HCT-116 cells and RBM39 G268V knock-in cells. Errors bars represent SEM (n=3).

FIG. 6. Indisulam treatment leads to splicing defects and DCAF15 expression is correlated with indisulam sensitivity.

(A) 2 μM indisulam treatment for 6 and 12 hours results in abundant intron retention and exon skipping events in HCT-116 cells.
(B) An example of exon skippings found in TRIM27. Red arrows indicate skipped exons.
(C) An example of intron retentions observed in EZH2.
(D) Cell lines of the hematopoietic and lymphoid (HL) origin are more sensitive to indisulam (p<0.0001 by Mann Whitney U-Test).
(E) DCAF15 expression is negatively correlated with indisulam AUC in HL cell lines.
(F) DCAF15 copy number is negatively correlated with indisulam AUC in HL cell lines.

FIG. 7. Carbonic anhydrase inhibitors are not toxic to HCT-116 cells.

(A) Structures of two canonical carbonic anhydrases, topiramate and acetazolamide.
(B) Dose response curves of indisulam, topiramate, and acetazolamide in HCT-116 cells.

FIG. 8. Characterization of indisulam resistant clones.

(A) indisulam resistant clones are not resistant to paclitaxel.
(B) Sanger sequencing of cDNA confirms RBM39 mutations (from top to bottom, SEQ ID NOs: 14-23).
(C) RBM39 mutations identified in a collection of 19 clones.

FIG. 9. RBM39 G268V knock-in using CRISPR/Cas9 technology.

(A) Genomic target (+strand, from left to right: SEQ ID NOs: 24-26;—strand, from left to right: ID NOs: 27-29) and ssODN repair template sequences (from left to right: SEQ ID NOs: 30-32).
(B) Including the ssODN repair template increases rates of indisulam resistance, visualized by crystal violet staining.
(C) Confirmation of G268V genomic conversion in isolated clones by Sanger sequencing (from top to bottom: SEQ ID NOs: 33-38).
(D) Indisulam IC50 measurements of parental and RBM39 G268V knock-in H1155 cells.

FIG. 10. Characterization of indisulam dependent degradation of RBM39

(A) Dose dependent degradation of RBM39 in parental HCT-116 cells, but not in cells harboring the RBM39 G268V mutation. Cells were treated with indicated concentrations of indisulam for 12 hours before being lysed for western blotting.
(B) Time course of indisulam dependent RBM39 degradation. Cells were treated with 2 μM indisulam for the indicated time before being lysed for western blotting.
(C) qPCR quantification of RBM39 mRNA levels in HCT-116 cells exposed to different concentrations of indisulam.
(D) Homology directed repair facilitates tagging of endogenous RBM39 by AID, 3xFLAG, AcGFP, or Nanoluciferase

FIG. 11. Characterization of resistant cell lines to bortezomib and MLN4924.

(A) Bortezomib dose response curves of parental HCT-116 cells and a bortezomib resistant HCT-116 clone.
(B) Sanger sequencing confirms the presence of an A108T mutation in PSMB5 in the bortezomib resistant HCT-116 clone (SEQ ID NO: 39).
(C) MLN4924 dose response curves of parental HCT-116 cells and an MLN4924 resistant HCT-116 clone.
(D) Sanger sequencing confirms the presence of an A171T mutation in UBA3 in the MLN4924 resistant HCT-116 clone (SEQ ID NO: 40).

FIG. 12. Characterization of C-terminally tagged RBM39.

(A) 3xFLAG tag preserves indisulam dependent degradation of RBM39 in HCT-116 cells. RBM39 G268V-3xFLAG is not degraded following indisulam treatment.
(B) AcGFP tagging results in an increase in molecular weight of RBM39, and preserves indisulam dependent degradation of RBM39 in H1155 cells.
(C) NanoLuc tagging results in an increase in molecular weight of RBM39 in H1155 cells.

FIG. 13. Characterization of DCAF15−/− cells.

(A) Sanger sequencing confirms inactivation of DCAF15 (from top to bottom: SEQ ID NOs: 41-45).
(B) Expression of DCAF15-3xFLAG cDNA restores indisulam dependent degradation of RBM39 in DCAF15−/− cells.
(C) Expression of DCAF15-3xFLAG cDNA restores indisulam sensitivity in DCAF15−/− cells.

FIG. 14. Staining of purified proteins.

(A) Coomassie blue staining of RBM39Δ150 expressed and purified from Sf9 cells.
(B) Silver staining of DDB1/DCAF15-3xFLAG expressed and purified from 293T cells.
(C) Silver staining of RBM39-3xFLAG expressed and purified from HCT-116 cells.

FIG. 15. Indisulam dependent formation of a soluble DDB1/DCAF15/RBM39A150 complex.

(A) DCAF15-GST/DDB1 and RBM39 migrate as separate gel filtration peaks.
(B) RBM39Δ150 co-migrates with DCAF15-GST/DDB1 in the presence of indisulam.
(C) Indisulam does not change the migration pattern of DDB1/DCAF15-GST.
(D) Indisulam does not change the migration pattern of RBM39Δ150.

FIG. 16. RBM39 interacts with splicing factors.

(A) Silver staining of proteins purified by Anti-Flag antibodies from lysate collected from either H1155 parental or H1155 RBM39-3XFLAG expressing cells.
(B) RBM39 interacting proteins with greater than 20 peptide counts discovered by mass spectrometry.

FIG. 17. An illustration of how Fisher's exact test was used to identify exon skipping, intron retention, and alternative splice sites.

FIG. 18. Characterization of splicing changes by PCR.

(A) Exon skipping of TRIM27 in an indisulam dose dependent manner. Black arrow indicates full length PCR product with all exons present, and red arrows indicate PCR products with 1 or 2 skipped exons.
(B) Two independent siRNAs efficiently knock down RBM39.
(C) Exon skipping of TRIM27 observed in RBM39 siRNA treated cells.
(D) Intron retention observed in RBM3.
(E) Intron retention in RBM3 in an indisulam dose dependent manner. In comparison, spliced RBM3 RNAs levels are not affected.
(F) Intron retention in RBM3 observed in RBM39 siRNA treated cells.

FIG. 19. Sanger sequencing of a RT-PCR product from both HCC78 (SEQ ID NO: 46) and H1373 (SEQ ID NO: 47) cell lines show a heterozygous mutation (C to T) in U2AF1 leading to a Serine to Phenylalanine mutation at position 34.

FIG. 20. Viability assay show cancer cell lines harboring a mutation in U2AF1 are sensitive to Indisulam.

FIG. 21. IC50 assay showing cancer cell lines that express U2AF1 S34F mutations are significantly more sensitive to indisulam than cell lines that express U2AF1 wild type

Table 1. Proteins identified in RBM39 complex+/−indisulam.
Table 2. Proteins identified in RBM39 complex vs. control.
Table 3. Correlations between gene expression and indisulam sensitivity in hematopoetic and lymphoid cancer cell lines.
Table 4. Correlations between gene copy number and indisulam sensitivity in hematopoetic and lymphoid cancer cell lines.
Table 5. IC50 data for WT non-small cell lung cancer cell lines or non-small cell lung cancer cell lines with U2AF1 mutations.

### DETAILED DESCRIPTION

The present disclosure, in certain embodiments, is based on the surprising discovery that clinically active aryl sulfonamides (e.g., indisulam, tasisulam and chloroquinoxaline sulfonamide (CQS)) target pre-mRNA splicing in cancer through a mechanism of action analogous to IMiDs. These sulfonamides target the pre-mRNA splicing factor RBM39 for proteasomal degradation by recruiting CUL4-DCAF15. The anti-cancer activity of clinically tested sulfonamides targets an essential splicing factor RBM39 for proteasomal degradation by recruiting the E3 ubiquitin ligase receptor DCAF15. Thus, DCAF15 can be used as a predicative biomarker for guiding the use of indisulam and other aryl sulfonamides. Furthermore, mutations in splicing factors such as U2AF1, SF3B1, SRSF2, and ZRSR2 can also be used as a cancer biomarker, e.g., together with the DCAF15 biomarker.

### Definitions

Unless defined otherwise, all technical and scientific terms used herein have the meaning commonly understood by one of ordinary skill in the art to which the disclosure pertains.

As used in the specification and the appended claims, the singular forms “a,” “an,” and “the” include plural referents unless the context clearly dictates otherwise. Thus, for example, references to “the method” includes one or more methods, and/or steps of the type described herein and/or which will become apparent to those persons skilled in the art upon reading this disclosure and so forth.

As used herein, the term “about” means within 20%, more preferably within 10% and most preferably within 5%.

An “analogue” of, e.g., indisulam, tasisulam and CQS, refers to a chemical species that retains substantially the same pharmacophore (a description of the main molecular features necessary for biological activity and their relative positions in space) as a lead compound. One or ordinary skill in the medicinal chemistry would be able to perform routine structure-activity relationship analysis to identify the pharmacophore and design suitable analogues. See, e.g., Drug Discov Today. 2006 April; 11(7-8):348-54 and Toxicol. Sci. (2000) 56 (1): 8-17, both incorporated herein by reference in their entirety.

The terms “cancer” and “cancerous” refer to or describe the physiological condition in mammals that is typically characterized by unregulated cell growth. Included in this definition are benign and malignant cancers. Examples of cancer include, but are not limited to, carcinoma, lymphoma, blastoma (including medulloblastoma and retinoblastoma), sarcoma (including liposarcoma and synovial cell sarcoma), neuroendocrine tumors (including carcinoid tumors, gastrinoma, and islet cell cancer), mesothelioma, schwannoma (including acoustic neuroma), meningioma, adenocarcinoma, melanoma, and leukemia or lymphoid malignancies. More particular examples of such cancers include squamous cell cancer (e.g., epithelial squamous cell cancer), lung cancer including small-cell lung cancer (SCLC), non-small cell lung cancer (NSCLC), adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer (including metastatic breast cancer), colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, testicular cancer, esophageal cancer, tumors of the biliary tract, as well as head and neck cancer. In some embodiments, “cancer” can be leukemias and lymphomas having the highest expression of DCAF15. In certain embodiments, the cancer can be myelodysplastic syndrome, chronic lymphocytic leukemia, and acute myeloid leukemia.

As used herein the term “comprising” or “comprises” is used in reference to compositions, methods, and respective component(s) thereof, that are present in a given embodiment, yet open to the inclusion of unspecified elements.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

DCAF15 refers to DDB1- and CUL4-Associated Factor 15, a substrate-specific receptor. The CUL4-DDB1 ubiquitin ligase regulates cell proliferation, survival, DNA repair, and genomic, integrity through targeted ubiquitination of key regulators. In some embodiments, the DCAF15 is from Homo sapiens, located on Chromosome 19, NC_000019.10 (13952507.13961444) and having RefSeq accession number NM_138353 (mRNA) or NP_612362 (protein).

RBM39 refers to RNA binding motif protein 39. In some embodiments, the RBM39 is from Homo sapiens, having RefSeq accession number NG_029955 (gene), NM_004902 (mRNA) or NP_004893 (protein).

An “effective amount” or “therapeutically effective amount” refers to an amount of a compound that confers a therapeutic effect (e.g., treats, controls, relieves, ameliorates, alleviates, slows the progression of, prevents, or delays the onset of or reduces the risk of developing, a disease, disorder, or condition or symptoms thereof) on the treated subject. The therapeutic effect may be objective (i.e., measurable by some test or marker) or subjective (i.e., subject gives an indication of or feels an effect). An effective amount of the compound described herein may range from about 0.0001 mg/kg to about 1000 mg/kg (e.g., from about 0.001 mg/kg to about 100 mg/kg, from about 0.01 mg/kg to about 100 mg/kg). Effective doses will also vary depending on route of administration, as well as the possibility of co-usage with other agents.

As used herein, the term “patient” or “individual” or “subject” refers to any person or mammalian subject for whom or which therapy is desired, and generally refers to the recipient of the therapy to be practiced according to the disclosure.

By “pharmaceutically acceptable” is meant a material that is not biologically or otherwise undesirable, i.e., the material may be incorporated into a pharmaceutical composition administered to a patient without causing any undesirable biological effects or interacting in a deleterious manner with any of the other components of the composition in which it is contained. When the term “pharmaceutically acceptable” is used to refer to a pharmaceutical carrier or excipient, it is implied that the carrier or excipient has met the required standards of toxicological and manufacturing testing and/or that it is included on the Inactive Ingredient Guide prepared by the U.S. Food and Drug administration.

The term “pro-drug” or “prodrug” refers to a compound that requires chemical conversion in tissue, plasma or tumor to be converted into an active drug. This process can be medated by an enzyme, for example a cytochrome P450 enzyme, and could involve addition of oxygen atoms or the cleavage of certain groups.

“Sample” or “biological sample” includes sections of tissues such as biopsy and autopsy samples, and frozen sections taken for histologic purposes. Such samples include blood, sputum, tissue, lysed cells, brain biopsy, cultured cells, e.g., primary cultures, explants, and transformed cells, stool, urine, etc. A biological sample is typically obtained from a eukaryotic organism, most preferably a mammal such as a primate, e.g., chimpanzee or human; cow; dog; cat; a rodent, e.g., guinea pig, rat, mouse; rabbit; or a bird; reptile; or fish. For the purpose of diagnosing cancer or predicting responsiveness of cancer to a treatment, the sample may or may not contain cancerous cells. A “control sample” is typically a sample which does not contain cancerous cells (e.g., a sample from benign tissues), or a sample which does not exhibit elevated DCAF15 levels or activity (including samples from benign or cancerous tissues, or histologically normal tissue adjacent but outside the margin of tumors). Non-limiting examples of control samples for use in the current disclosure include, non-cancerous tissue extracts, surgical margins extracted from the subject, isolated cells known to have normal DCAF15 levels, obtained from the subject under examination or other healthy individuals. In one aspect, the control sample of the present disclosure is benign tissue. In one embodiment of the current disclosure, the amount of DCAF15 in a sample is compared to either a standard amount of DCAF15 present in a normal cell or a non-cancerous cell, or to the amount of DCAF15 in a control sample. The comparison can be done by any method known to a skilled artisan.

As used herein, “selective” or “selectivity” generally means that a compound is selectively toxic towards some, but not all, cancer cells, cancer cell lines and/or cancer types. In some embodiments, the aryl sulfonamide compounds (or pharmaceutically acceptable salts or prodrugs thereof) described herein can be used to target specific anomalies (e.g., genetic, epigenetic and/or metabolic) that lead to DCAF15 gene amplification and/or mRNA/protein overexpression. Selectivity may be measured by the half maximal inhibitory concentration (IC₅₀), which, as used herein, is a measure of the effectiveness of a compound in killing cells. This quantitative measure indicates how much of a particular compound is needed to kill a particular cell population by half (e.g., as indicated by the amount of ATP or cell survival). In other words, it is the half maximal (50%) inhibitory concentration (IC) of a substance (50% IC, or IC₅₀). The IC₅₀ can be determined by constructing a dose-response curve. The lower the IC₅₀, the higher the potency, and the greater the difference between IC₅₀ values for different cells or cell lines (e.g., sensitive lines vs. insensitive lines, and sensitive lines vs. normal lines), the greater the selectivity. Generally, IC₅₀ lower than 5 μM (e.g., in the range of 1-100 nM) indicates high selectivity.

“Splicing factor” refers to the protein factors involved in the splicing of pre-mRNA taking place on a large ribonucleoprotein particle called the spliceosome, which comprises five snRNAs (small nuclear RNAs), U1, U2, U4, U5 and U6, and many other protein factors. Certain splicing factors are reviewed by Chen et al., Biosci Rep. 2012 Aug. 1; 32(Pt 4): 345-359, incorporated herein by reference in its entirety. Exemplary splicing factors include U2AF1 (U2 small nuclear RNA auxillary factor 1), a component of the U2 snRNP complex of the spliceosome; SF3B1 (splicing factor 3b subunit 1), SRSF2 (serine and arginine rich splicing factor 2), and ZRSR2 (U2 small nuclear ribonucleoprotein auxiliary factor 35 kDa subunit-related protein 2). In some embodiments, mutations in splicing factors can be used as biomarkers, in a DCAF15 independent manner or together with the DCAF15 biomarker, for the diagnosis, prognosis and/or selective treatment (e.g., using aryl sulfonamides) of cancer.

The terms “treating” and “treatment” as used herein refer to reduction in severity and/or frequency of symptoms, elimination of symptoms and/or underlying cause, and improvement or remediation of damage.

Other terms used in the fields of medicinal chemistry, recombinant nucleic acid technology, microbiology, immunology, antibody engineering, and molecular and cell biology as used herein will be generally understood by one of ordinary skill in the applicable arts. For example, conventional techniques may be used for preparing recombinant DNA, performing oligonucleotide synthesis, and practicing tissue culture and transformation (e.g., electroporation, transfection or lipofection). Enzymatic reactions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification. See, e.g., Sambrook et al., 2001, Molecular Cloning: A Laboratory Manual, 3rd ed., Cold Spring Harbor Laboratory Press, Cold Spring Harbor, N.Y., which is incorporated herein by reference for any purpose. Unless specific definitions are provided, the nomenclature utilized in connection with, and the laboratory procedures and techniques of, analytical chemistry, synthetic organic chemistry, and medicinal and pharmaceutical chemistry described herein are those well-known and commonly used in the art. Standard techniques may be used for chemical syntheses, chemical analyses, pharmaceutical preparation, formulation, and delivery, and treatment of patients.

Various aspects and embodiments are described in further detail in the following subsections.

### Compounds, Forms and Salts

Exemplary compounds of the present disclosure include aryl sulfonamides (e.g., indisulam, tasisulam and chloroquinoxaline sulfonamide (CQS)). Also included herein are analogues of indisulam, tasisulam and CQS. For example, one or ordinary skill in the medicinal chemistry would be able to perform routine structure-activity relationship analysis, thereby identifying the pharmacophore therein and designing suitable analogues thereof. More specific examples of indisulam analogues are disclosed in PCT Publication Nos. WO1995007276A1, WO2002042493A1, and WO2006036025A1; exemplary tasisulam analogues are disclosed in U.S. Pat. Nos. 5,302,724, 7,084,170 and 7,250,430; and exemplary CQS analogues are disclosed in U.S. Pat. Nos. 4,931,433, 5,529,999, and 6,787,534; all of the foregoing publications are hereby incorporated herein by reference in their entirety.

The compounds described herein may contain one or more asymmetric centers and thus occur as racemates and racemic mixtures, enantiomerically enriched mixtures, single enantiomers, individual diastereomers and diastereomeric mixtures. All such isomeric forms of these compounds are expressly included in the present disclosure. The compounds of the present disclosure may also contain linkages (e.g., carbon-carbon bonds, carbon-nitrogen bonds such as amide bonds) wherein bond rotation is restricted about that particular linkage, e.g. restriction resulting from the presence of a ring or double bond. Accordingly, all cis/trans and E/Z isomers and rotational isomers are expressly included in the present disclosure. The compounds of the present disclosure may also be represented in multiple tautomeric forms, in such instances, the present disclosure expressly include all tautomeric forms of the compounds described herein, even though only a single tautomeric form may be represented. All such isomeric forms of such compounds are expressly included in the present disclosure.

Optical isomers can be obtained in pure form by standard procedures known to those skilled in the art, and include, but are not limited to, diastereomeric salt formation, kinetic resolution, and asymmetric synthesis. See, for example, Jacques, et al., Enantiomers, Racemates and Resolutions (Wiley Interscience, New York, 1981); Wilen, S. H., et al., Tetrahedron 33:2725 (1977); Eliel, E. L. Stereochemistry of Carbon Compounds (McGraw-Hill, N Y, 1962); Wilen, S. H. Tables of Resolving Agents and Optical Resolutions p. 268 (E. L. Eliel, Ed., Univ. of Notre Dame Press, Notre Dame, Ind. 1972), each of which is incorporated herein by reference in their entireties. It is also understood that the present disclosure encompass all possible regioisomers, and mixtures thereof, which can be obtained in pure form by standard separation procedures known to those skilled in the art, and include, but are not limited to, column chromatography, thin-layer chromatography, and high-performance liquid chromatography.

The compounds of the present disclosure include the compounds themselves, as well as their salts and their prodrugs, if applicable. A salt, for example, can be formed between an anion and a positively charged substituent (e.g., amino) on a compound described herein. Suitable anions include chloride, bromide, iodide, sulfate, nitrate, phosphate, citrate, methanesulfonate, trifluoroacetate, and acetate. Likewise, a salt can also be formed between a cation and a negatively charged substituent (e.g., carboxylate) on a compound described herein. Suitable cations include sodium ion, potassium ion, magnesium ion, calcium ion, and an ammonium cation such as tetramethylammonium ion. Examples of prodrugs include C₁₋₆ alkyl esters of carboxylic acid groups, which, upon administration to a subject, are capable of providing active compounds.

Pharmaceutically acceptable salts of the compounds of the present disclosure include those derived from pharmaceutically acceptable inorganic and organic acids and bases. As used herein, the term “pharmaceutically acceptable salt” refers to a salt formed by the addition of a pharmaceutically acceptable acid or base to a compound disclosed herein. As used herein, the phrase “pharmaceutically acceptable” refers to a substance that is acceptable for use in pharmaceutical applications from a toxicological perspective and does not adversely interact with the active ingredient.

Examples of suitable acid salts include acetate, adipate, alginate, aspartate, benzoate, benzenesulfonate, bisulfate, butyrate, citrate, camphorate, camphorsulfonate, digluconate, dodecylsulfate, ethanesulfonate, formate, fumarate, glucoheptanoate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, palmoate, pectinate, persulfate, 3-phenylpropionate, phosphate, picrate, pivalate, propionate, salicylate, succinate, sulfate, tartrate, thiocyanate, tosylate and undecanoate. Other acids, such as oxalic, while not in themselves pharmaceutically acceptable, may be employed in the preparation of salts useful as intermediates in obtaining the compounds of the present disclosure and their pharmaceutically acceptable acid addition salts. Salts derived from appropriate bases include alkali metal (e.g., sodium), alkaline earth metal (e.g., magnesium), ammonium and N-(alkyl)₄⁺ salts. The present disclosure also envisions the quaternization of any basic nitrogen-containing groups of the compounds disclosed herein. Water or oil-soluble or dispersible products may be obtained by such quaternization. Salt forms of the compounds of any of the formulae herein can be amino acid salts of carboxyl groups (e.g. L-arginine, -lysine, -histidine salts).

Lists of suitable salts are found in Remington's Pharmaceutical Sciences, 17th ed., Mack Publishing Company, Easton, Pa., 1985, p. 1418; Journal of Pharmaceutical Science, 66, 2 (1977); and “Pharmaceutical Salts: Properties, Selection, and Use A Handbook; Wermuth, C. G. and Stahl, P. H. (eds.) Verlag Helvetica Chimica Acta, Zurich, 2002 [ISBN 3-906390-26-8] each of which is incorporated herein by reference in their entireties.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present disclosure.

In addition to salt forms, the present disclosure provides compounds which are in a prodrug form. Prodrugs of the compounds described herein are those compounds that undergo chemical changes under physiological conditions to provide the compounds of the present disclosure. Additionally, prodrugs can be converted to the compounds of the present disclosure by chemical or biochemical methods in an ex vivo environment. For example, prodrugs can be slowly converted to the compounds of the present disclosure when placed in a transdermal patch reservoir with a suitable enzyme or chemical reagent. Prodrugs are often useful because, in some situations, they may be easier to administer than the parent drug. They may, for instance, be more bioavailable by oral administration than the parent drug. The prodrug may also have improved solubility in pharmacological compositions over the parent drug. A wide variety of prodrug derivatives are known in the art, such as those that rely on hydrolytic cleavage or oxidative activation of the prodrug. An example, without limitation, of a prodrug would be a compound of the present disclosure which is administered as an ester (the “prodrug”), but then is metabolically hydrolyzed to the carboxylic acid, the active entity. Additional examples include peptidyl derivatives of a compound of the present disclosure.

The present disclosure also includes various hydrate and solvate forms of the compounds.

The compounds of the present disclosure may also contain unnatural proportions of atomic isotopes at one or more of the atoms that constitute such compounds. For example, the compounds may be radiolabeled with radioactive isotopes, for example tritium (³H), iodine-125 (¹²⁵I) or carbon-14 (¹⁴C). All isotopic variations of the compounds of the present disclosure, whether radioactive or not, are intended to be encompassed within the scope of the present disclosure.

### Pharmaceutical Compositions

The term “pharmaceutically acceptable carrier” refers to a carrier or adjuvant that may be administered to a subject (e.g., a patient), together with a compound of the present disclosure, and which does not destroy the pharmacological activity thereof and is nontoxic when administered in doses sufficient to deliver a therapeutic amount of the compound.

Pharmaceutically acceptable carriers, adjuvants and vehicles that may be used in the compositions of the present disclosure include, but are not limited to, ion exchangers, alumina, aluminum stearate, lecithin, self-emulsifying drug delivery systems (SEDDS) such as d-α-tocopherol polyethyleneglycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts, or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as α-, β-, and γ-cyclodextrin, or chemically modified derivatives such as hydroxyalkylcyclodextrins, including 2- and 3-hydroxypropyl-β-cyclodextrins, or other solubilized derivatives may also be advantageously used to enhance delivery of compounds of the formulae described herein.

The compositions for administration can take the form of bulk liquid solutions or suspensions, or bulk powders. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules, losenges or the like in the case of solid compositions. In such compositions, the compound is usually a minor component (from about 0.1 to about 50% by weight or preferably from about 1 to about 40% by weight) with the remainder being various vehicles or carriers and processing aids helpful for forming the desired dosing form.

The pharmaceutical composition of the present invention can be prepared by blending the compound of the present invention as an active ingredient, a pharmaceutically acceptable carrier and if needed an additive, and formulated into a dosage form. Specific examples of the dosage form include oral preparations such as tablets, coated tablets, pills, powders, granules, capsules, solutions, suspensions and emulsions; and parenteral preparations such as injections, infusions, suppositories, ointments and patches. The blending ratio of the carrier or the additive is appropriately determined based on the range of the blending ratio conventionally adopted in the pharmaceutical field. The carrier or the additive that can be blended is not particularly limited, and examples thereof include water, physiological saline and other aqueous solvents; various carriers such as aqueous bases and oily bases; and various additives such as excipients, binders, pH adjusters, disintegrants, absorption enhancers, lubricants, colorants, corrigents and fragrances.

Examples of the additive that can be blended into tablets, capsules and the like include binders such as gelatin, cornstarch, tragacanth and gum arabic; excipients such as crystalline cellulose; bulking agents such as cornstarch, gelatin and alginic acid; lubricants such as magnesium stearate; sweeteners such as sucrose, lactose and saccharin; and flavors such as peppermint, Gaultheria adenothrix oil and cherry. In the case where the unit dosage form is a capsule, a liquid carrier such as fats and oils can be further blended in addition to the above-mentioned materials. A sterile composition for injection can be prepared according to an ordinary pharmaceutical formulation practice, for example, by dissolving or suspending an active substance in a vehicle such as water for injection and a natural vegetable oil (such as sesame oil and coconut oil). As an aqueous liquid for injection, for example, physiological saline, an isotonic solution containing glucose and an auxiliary substance (for example, D-sorbitol, D-mannitol, sodium chloride, etc.), or the like can be used, optionally together with a suitable solubilizer such as alcohols (for example, ethanol), polyalcohols (for example, propylene glycol, polyethylene glycol) and nonionic surfactants (for example, polysorbate 80™, HCO-50). As an oily liquid, for example, sesame oil, soybean oil or the like can be used, optionally together with a solubilizer such as benzyl benzoate and benzyl alcohol. Further, a buffering agent (for example, a phosphate buffer, a sodium acetate buffer), a soothing agent (for example, benzalkonium chloride, procaine hydrochloride, etc.), a stabilizer (for example, human serum albumin, polyethylene glycol, etc.), a preservative (for example, benzyl alcohol, phenol, etc.), an antioxidant etc. may also be blended.

The pharmaceutical preparation that can be obtained in the above manner is safe and less toxic, and therefore can be administered to, for example, humans and other mammals (rats, mice, rabbits, sheep, pigs, cows, cats, dogs, monkeys, etc.).

### Administration

The compounds and compositions described herein can, for example, be administered orally, parenterally (e.g., subcutaneously, intracutaneously, intravenously, intramuscularly, intraarticularly, intraarterially, intrasynovially, intrasternally, intrathecally, intralesionally and by intracranial injection or infusion techniques), by inhalation spray, topically, rectally, nasally, buccally, vaginally, via an implanted reservoir, by injection, subdermally, intraperitoneally, transmucosally, or in an ophthalmic preparation, with a dosage ranging from about 0.01 mg/kg to about 1000 mg/kg, (e.g., from about 0.01 to about 100 mg/kg, from about 0.1 to about 100 mg/kg, from about 1 to about 100 mg/kg, from about 1 to about 10 mg/kg) every 4 to 120 hours, or according to the requirements of the particular drug. The interrelationship of dosages for animals and humans (based on milligrams per meter squared of body surface) is described by Freireich et al., Cancer Chemother. Rep. 50, 219 (1966). Body surface area may be approximately determined from height and weight of the patient. See, e.g., Scientific Tables, Geigy Pharmaceuticals, Ardsley, N.Y., 537 (1970). In certain embodiments, the compositions are administered by oral administration or administration by injection. The methods herein contemplate administration of an effective amount of compound or compound composition to achieve the desired or stated effect. Typically, the pharmaceutical compositions of the present disclosure will be administered from about 1 to about 6 times per day or alternatively, as a continuous infusion. Such administration can be used as a chronic or acute therapy.

Lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

Upon improvement of a patient's condition, a maintenance dose of a compound, composition or combination of the present disclosure may be administered, if necessary. Subsequently, the dosage or frequency of administration, or both, may be reduced, as a function of the symptoms, to a level at which the improved condition is retained when the symptoms have been alleviated to the desired level. Patients may, however, require intermittent treatment on a long-term basis upon any recurrence of disease symptoms.

The amount administered depends on the compound formulation, route of administration, etc. and is generally empirically determined in routine trials, and variations will necessarily occur depending on the target, the host, and the route of administration, etc. Generally, the quantity of active compound in a unit dose of preparation may be varied or adjusted from about 1, 3, 10 or 30 to about 30, 100, 300 or 1000 mg, according to the particular application. In a particular embodiment, unit dosage forms are packaged in a multipack adapted for sequential use, such as blisterpack, comprising sheets of at least 6, 9 or 12 unit dosage forms. The actual dosage employed may be varied depending upon the requirements of the patient and the severity of the condition being treated. Determination of the proper dosage for a particular situation is within the skill of the art. Generally, treatment is initiated with smaller dosages which are less than the optimum dose of the compound. Thereafter, the dosage is increased by small amounts until the optimum effect under the circumstances is reached. For convenience, the total daily dosage may be divided and administered in portions during the day if desired.

The dose may vary depending on patient's state, the cancer type, the condition, the administration method and the like, but in general, the daily oral dose for a human weighing about 60 kg is, for example, about 0.1 to 1000 mg, preferably about 1.0 to 500 mg, and more preferably about 3.0 to 200 mg in terms of the active ingredient. As for the parenteral dose, the amount for one dose may vary depending on patient's state, the cancer type, the condition, the administration method and the like, but for example in the case of injections, it is usually advantageous that the active ingredient is intravenously administered in an amount of, for example, about 0.01 to 100 mg, preferably about 0.01 to 50 mg, and more preferably about 0.01 to 20 mg per kg body weight. The daily total dose may be a single dose or divided into several portions.

In some embodiments, the compounds described herein can be coadministered with one or more other therapeutic agents. In certain embodiments, the additional agents may be administered separately, as part of a multiple dose regimen, from the compounds of the present disclosure (e.g., sequentially, e.g., on different overlapping schedules with the administration of one or more compounds of formula (I) (including any subgenera or specific compounds thereof)). In other embodiments, these agents may be part of a single dosage form, mixed together with the compounds of the present disclosure in a single composition. In still another embodiment, these agents can be given as a separate dose that is administered at about the same time that one or more compounds of formula (I) (including any subgenera or specific compounds thereof) are administered (e.g., simultaneously with the administration of one or more compounds of formula (I) (including any subgenera or specific compounds thereof)). When the compositions of the present disclosure include a combination of a compound of the formulae described herein and one or more additional therapeutic or prophylactic agents, both the compound and the additional agent can be present at dosage levels of between about 1 to 100%, and more preferably between about 5 to 95% of the dosage normally administered in a monotherapy regimen.

According to the present invention, the medicament for cancer treatment can be used in combination with another cancer therapeutic drug. Such another cancer therapeutic drug is not particularly limited, but preferred is a chemotherapeutic drug, an immunotherapeutic drug or a hormone therapy drug, for example. According to the present invention, the medicament for cancer treatment can also be used in combination with radiotherapy.

The chemotherapeutic drug is not particularly limited and examples thereof include: alkylating agents such as nitrogen mustard, nitrogen mustard N-oxide hydrochloride, chlorambucil, cyclophosphamide, ifosfamide, thiotepa, carboquone, improsulfan tosilate, busulfan, nimustine hydrochloride, mitobronitol, melphalan, dacarbazine, ranimustine, estramustine phosphate sodium, triethylenemelamine, carmustine, lomustine, streptozocin, pipobroman, ethoglucid, carboplatin, cisplatin, miboplatin, nedaplatin, oxaliplatin, altretamine, ambamustine, dibrospidium chloride, fotemustine, prednimustine, pumitepa, Ribomustin, temozolomide, treosulfan, trofosfamide, zinostatin stimalamer, adozelesin, cystemustine and bizelesin; antimetabolites such as mercaptopurine, 6-mercaptopurine riboside, thioinosine, methotrexate, pemetrexed, enocitabine, cytarabine, cytarabine ocfosfate, ancitabine hydrochloride, 5-FU and its derivatives (for example, fluorouracil, tegafur, UFT, doxifluridine, carmofur, galocitabine, emitefur, capecitabine, etc.), aminopterin, nelzarabine, leucovorin calcium, Tabloid, butocin, calcium folinate, calcium levofolinate, cladribine, emitefur, fludarabine, gemcitabine, hydroxycarbamide, pentostatin, piritrexim, idoxuridine, mitoguazone, tiazofurin, ambamustine and bendamustine; anticancer antibiotics such as actinomycin D, actinomycin C, mitomycin C, chromomycin A3, bleomycin hydrochloride, bleomycin sulfate, peplomycin sulfate, daunorubicin hydrochloride, doxorubicin hydrochloride, aclarubicin hydrochloride, pirarubicin hydrochloride, epirubicin hydrochloride, neocarzinostatin, mithramycin, sarkomycin, carzinophilin, mitotane, zorubicin hydrochloride, mitoxantrone hydrochloride and idarubicin hydrochloride; and plant-derived anticancer drugs such as etoposide, etoposide phosphate, vinblastine sulfate, vincristine sulfate, vindesine sulfate, teniposide, paclitaxel, docetaxel and vinorelbine.

The immunotherapeutic drug is not particularly limited and examples thereof include picibanil, Krestin, sizofuran, lentinan, ubenimex, interferons, interleukins, macrophage colony-stimulating factor, granulocyte colony-stimulating factor, erythropoietin, lymphotoxins, BCG vaccine, Corynebacterium parvum, levamisole, polysaccharide K, procodazole, anti-PD1 antibody, anti-PD-L1 antibody, anti-EGFR antibody, and anti-CTLA4 antibody.

The hormone therapy drug is not particularly limited and examples thereof include fosfestrol, diethylstilbestrol, chlorotrianisene, medroxyprogesterone acetate, megestrol acetate, chlormadinone acetate, cyproterone acetate, danazol, allylestrenol, gestrinone, mepartricin, raloxifene, ormeloxifene, levormeloxifene, antiestrogens (for example, tamoxifen citrate, toremifene citrate, etc.), birth-control pills, mepitiostane, testololactone, aminoglutethimide, LH-RH agonists (for example, goserelin acetate, buserelin, leuprorelin, etc.), droloxifene, epitiostanol, ethinylestradiol sulfonate, aromatase inhibitors (for example, fadrozole hydrochloride, anastrozole, letrozole, exemestane, vorozole, formestane, etc.), antiandrogens (for example, flutamide, bicalutamide, nilutamide, etc.), 5α-reductase inhibitors (for example, finasteride, epristeride, etc.), corticosteroids (for example, dexamethasone, prednisolone, betamethasone, triamcinolone, etc.) and androgen synthesis inhibitors (for example, abiraterone, etc.).

The combined use of the medicament for cancer treatment, with another cancer therapeutic drug or radiotherapy, can provide the following effects without any limitation: (1) synergistic effect is obtainable; (2) the dose is reducible; (3) prolonged treatment period is selectable; and (4) persistent therapeutic effect can be expected.

In the case where the medicament for cancer treatment and another cancer therapeutic drug are used in combination, they may be simultaneously administered to a subject, or separately administered thereto at some interval. The dose of the drug in combined use can be determined based on its clinical dose and is appropriately selected depending on the subject, the age and body weight of the subject, the condition, the administration time, the dosage form, the administration method, the combination of drugs, etc.

The compositions of the present disclosure may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form.

The compositions may be in the form of a sterile injectable preparation, for example, as a sterile injectable aqueous or oleaginous suspension. This suspension may be formulated according to techniques known in the art using suitable dispersing or wetting agents (for example, Tween 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens or Spans and/or other similar emulsifying agents or bioavailability enhancers which are commonly used in the manufacture of pharmaceutically acceptable solid, liquid, or other dosage forms may also be used for the purposes of formulation.

The compositions of the present disclosure may be orally administered in any orally acceptable dosage form including, but not limited to, capsules, tablets, emulsions and aqueous suspensions, dispersions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions and/or emulsions are administered orally, the active ingredient may be suspended or dissolved in an oily phase is combined with emulsifying and/or suspending agents. If desired, certain sweetening and/or flavoring and/or coloring agents may be added.

The compositions of the present disclosure may also be administered in the form of suppositories for rectal administration. These compositions can be prepared by mixing a compound of the present disclosure with a suitable non-irritating excipient which is solid at room temperature but liquid at the rectal temperature and therefore will melt in the rectum to release the active components. Such materials include, but are not limited to, cocoa butter, beeswax and polyethylene glycols.

Topical administration of the compositions of the present disclosure is useful when the desired treatment involves areas or organs readily accessible by topical application. For application topically to the skin, the composition should be formulated with a suitable ointment containing the active components suspended or dissolved in a carrier. Carriers for topical administration of the compounds of the present disclosure include, but are not limited to, mineral oil, liquid petroleum, white petroleum, propylene glycol, polyoxyethylene polyoxypropylene compound, emulsifying wax and water. Alternatively, the composition can be formulated with a suitable lotion or cream containing the active compound suspended or dissolved in a carrier with suitable emulsifying agents. Suitable carriers include, but are not limited to, mineral oil, sorbitan monostearate, polysorbate 60, cetyl esters wax, cetearyl alcohol, 2-octyldodecanol, benzyl alcohol and water. The compositions of the present disclosure may also be topically applied to the lower intestinal tract by rectal suppository formulation or in a suitable enema formulation.

In some embodiments, topical administration of the compounds and compositions described herein may be presented in the form of an aerosol, a semi-solid pharmaceutical composition, a powder, or a solution. By the term “a semi-solid composition” is meant an ointment, cream, salve, jelly, or other pharmaceutical composition of substantially similar consistency suitable for application to the skin. Examples of semi-solid compositions are given in Chapter 17 of The Theory and Practice of Industrial Pharmacy, Lachman, Lieberman and Kanig, published by Lea and Febiger (1970) and in Remington's Pharmaceutical Sciences, 21st Edition (2005) published by Mack Publishing Company, which is incorporated herein by reference in its entirety.

Topically-transdermal patches are also included in the present disclosure. Also within the present disclosure is a patch to deliver active chemotherapeutic combinations herein. A patch includes a material layer (e.g., polymeric, cloth, gauze, bandage) and the compound of the formulae herein as delineated herein. One side of the material layer can have a protective layer adhered to it to resist passage of the compounds or compositions. The patch can additionally include an adhesive to hold the patch in place on a subject. An adhesive is a composition, including those of either natural or synthetic origin, that when contacted with the skin of a subject, temporarily adheres to the skin. It can be water resistant. The adhesive can be placed on the patch to hold it in contact with the skin of the subject for an extended period of time. The adhesive can be made of a tackiness, or adhesive strength, such that it holds the device in place subject to incidental contact, however, upon an affirmative act (e.g., ripping, peeling, or other intentional removal) the adhesive gives way to the external pressure placed on the device or the adhesive itself, and allows for breaking of the adhesion contact. The adhesive can be pressure sensitive, that is, it can allow for positioning of the adhesive (and the device to be adhered to the skin) against the skin by the application of pressure (e.g., pushing, rubbing) on the adhesive or device.

The compositions of the present disclosure may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

A composition having the compound of the formulae herein and an additional agent (e.g., a therapeutic agent) can be administered using any of the routes of administration described herein. In some embodiments, a composition having the compound of the formulae herein and an additional agent (e.g., a therapeutic agent) can be administered using an implantable device. Implantable devices and related technology are known in the art and are useful as delivery systems where a continuous, or timed-release delivery of compounds or compositions delineated herein is desired. Additionally, the implantable device delivery system is useful for targeting specific points of compound or composition delivery (e.g., localized sites, organs). Negrin et al., Biomaterials, 22(6):563 (2001). Timed-release technology involving alternate delivery methods can also be used in the present disclosure. For example, timed-release formulations based on polymer technologies, sustained-release techniques and encapsulation techniques (e.g., polymeric, liposomal) can also be used for delivery of the compounds and compositions delineated herein.

### EXAMPLES

The present disclosure will now be illustrated by reference to the following examples which set forth particularly embodiments. However, it should be noted that these embodiments are illustrative and are not to be construed as restricting the disclosure in any way.

Indisulam is an aryl sulfonamide drug with selective anti-cancer activity. Its mechanism of action and the basis for its selectivity are unknown. Here we show that indisulam promotes the recruitment of RBM39 (RNA binding motif protein 39) to the CUL4-DCAF15 E3 ubiquitin ligase, leading to RBM39 polyubiquitination and proteasomal degradation. Mutations in RBM39 that prevent its recruitment to CUL4-DCAF15 increase RBM39 stability and confer resistance to indisulam's cytotoxicity. RBM39 associates with pre-mRNA splicing factors, and inactivation of RBM39 by indisulam causes aberrant pre-mRNA splicing. Many cancer cell lines derived from hematopoietic and lymphoid lineages are sensitive to indisulam and their sensitivity correlates with DCAF15 expression levels. Two other clinically tested sulfanomides, tasisulam and CQS, share the same mechanism of action as indisulam. We propose that DCAF15 expression can be a useful biomarker to guide clinical trials of this class of drugs, which we refer to as SPLAMs (SPLicing inhibitor sulfonAMides).

Genomic analyses of human tumors have revealed that alterations in pre-mRNA splicing can contribute to cancer progression, suggesting that drugs targeting this process may be a valuable approach for cancer treatment (1). Many of the proteins important for pre-mRNA splicing have no enzymatic activity, however, and are thus challenging to target with small molecules (2). The discovery of the mechanism underlying the anti-tumor activity of thalidomide, lenalidomide and pomalidomide (collectively termed Immunomodulatory Drugs (IMiDs)) has provided a strategy to target otherwise undruggable proteins (3-6). IMiDs bind to Cereblon (CRBN), which is the substrate receptor for the E3 ubiquitin ligase complex CUL4-DDB1-RBX1-CRBN (CUL4-CRBN) (3). Binding to CRBN not only inhibits the endogenous E3 ubiquitin ligase activity of CUL4-CRBN but also repurposes the enzyme to ubiquitinate other proteins as neo-substrates. For example, the clinical activity of IMiDs in multiple myeloma is the result of ubiquitination and degradation of two transcription factors, IKZF1 (Ikaros) and IKZF3 (Aiolos) (5, 6). IMiDs are also used clinically for treatment of 5q deletion associated myelodysplastic syndrome and in that setting their efficacy is due to induced degradation of the CSNK1A1 (CK1α) protein kinase (6). Here, we have discovered that cancer drugs called aryl sulfonamides, which have shown efficacy in a subset of cancer patients, act by targeting pre-mRNA splicing through a mechanism analogous to that of IMiDs. These sulfonamides target the pre-mRNA splicing factor RBM39 for proteasomal degradation by recruiting it to CUL4-DCAF15 E3 ubiquitin ligase.

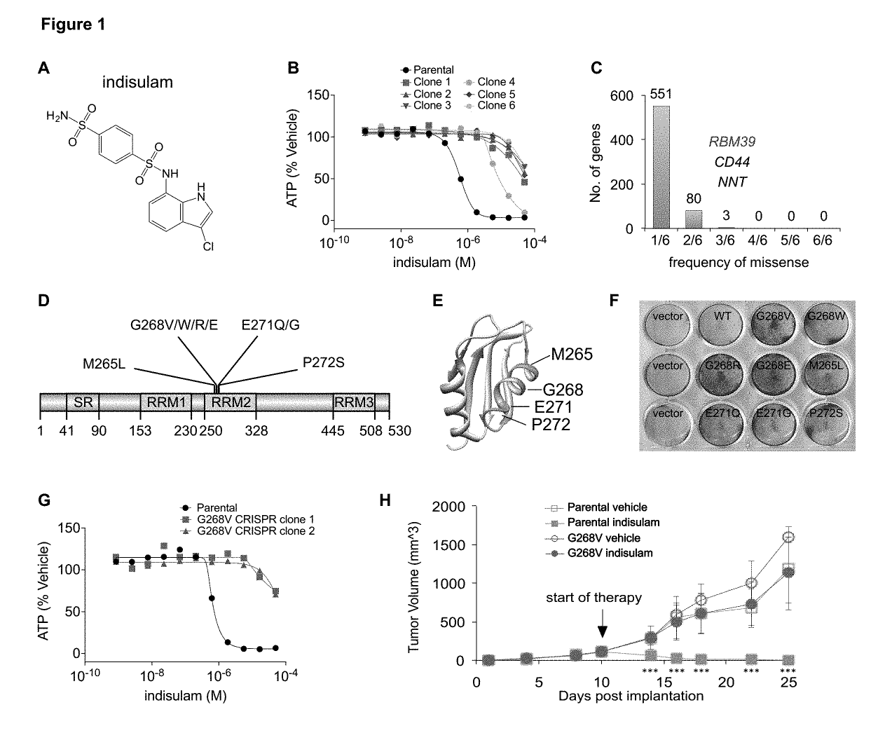

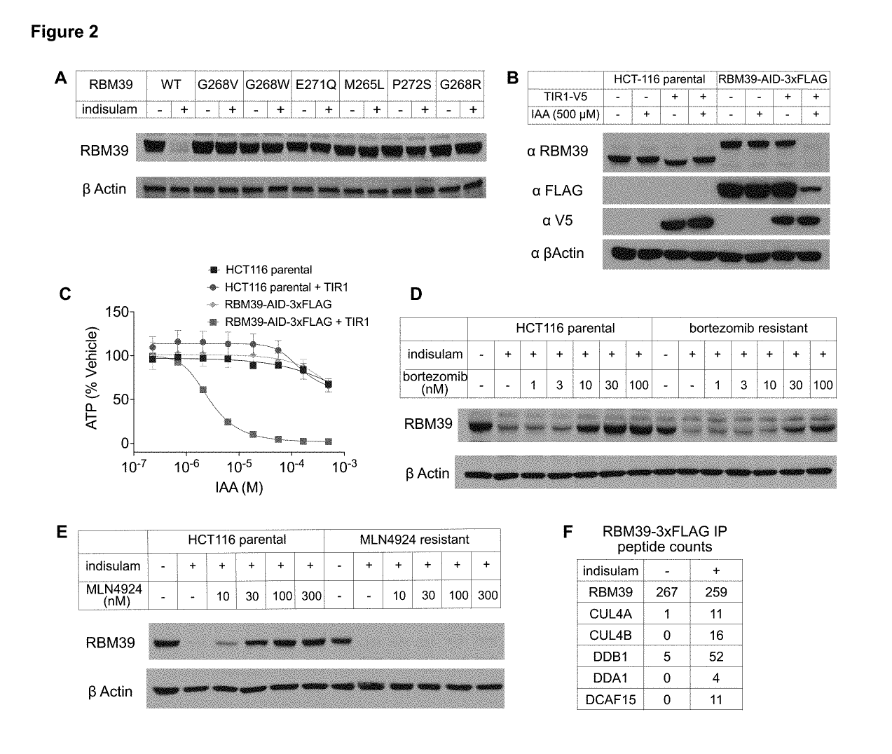

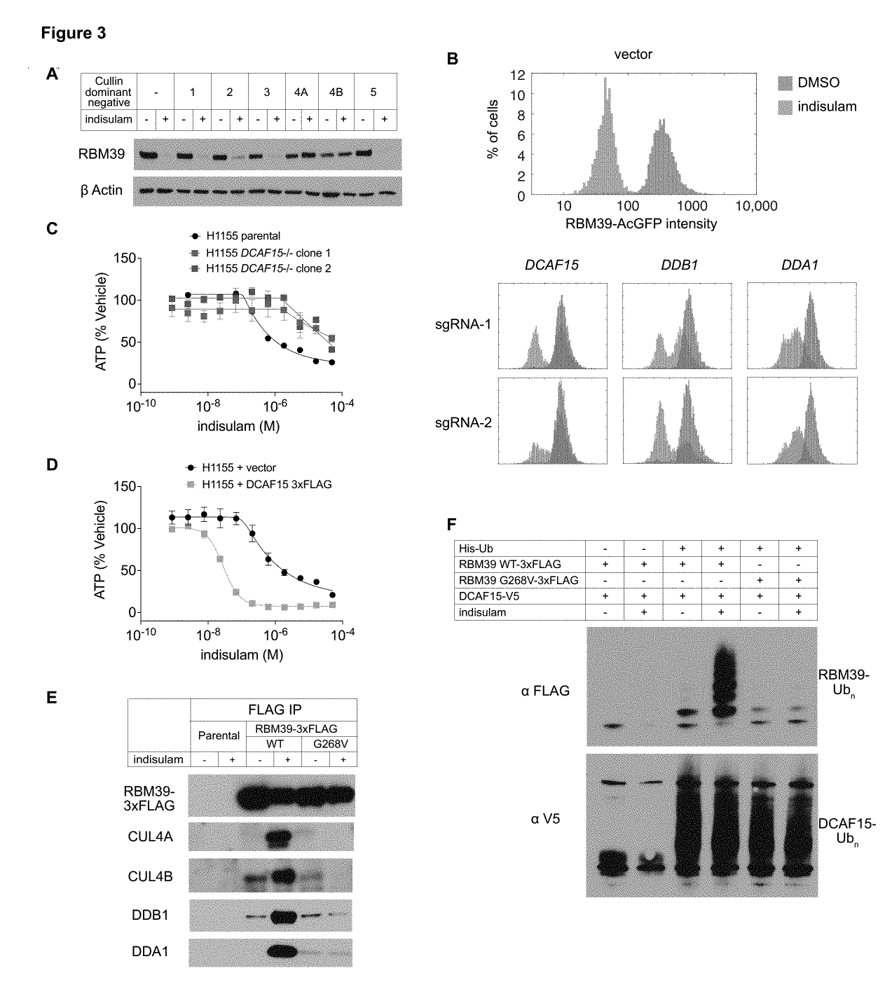

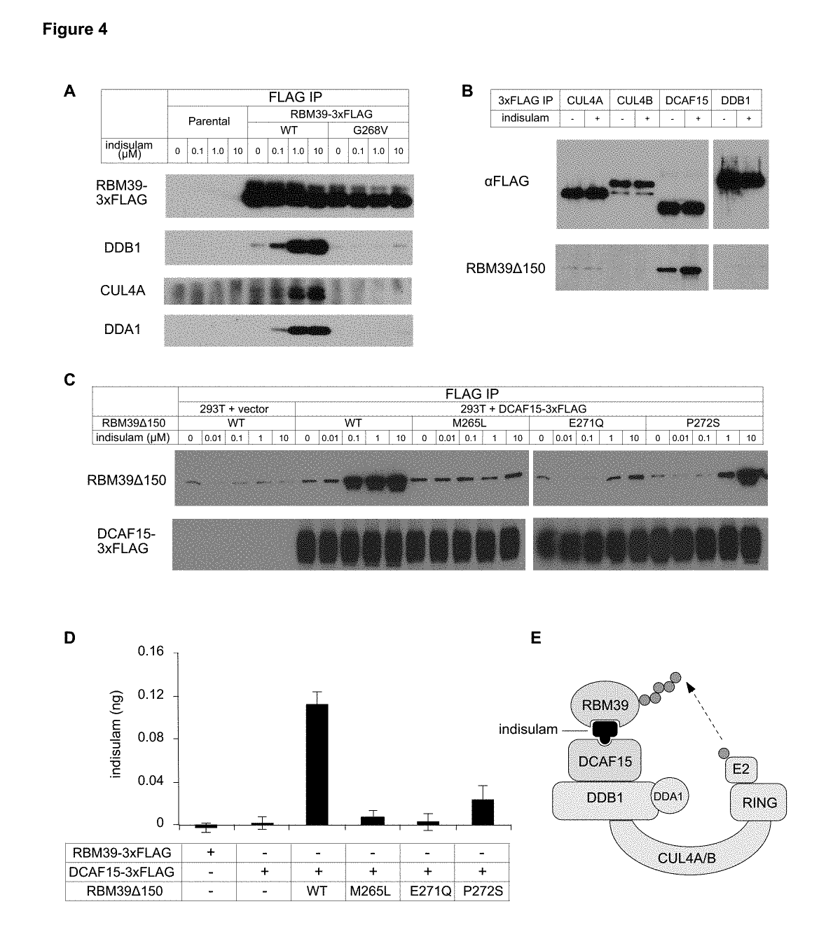

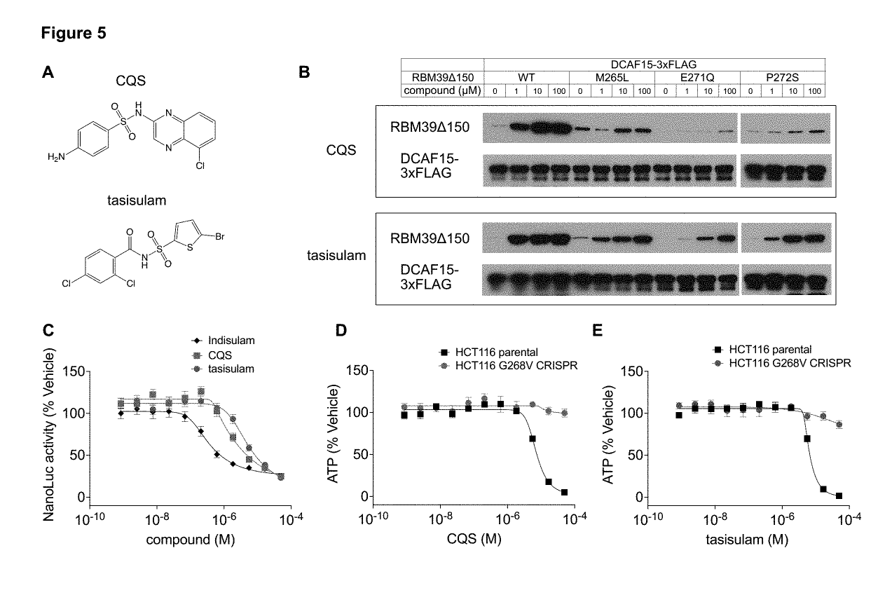

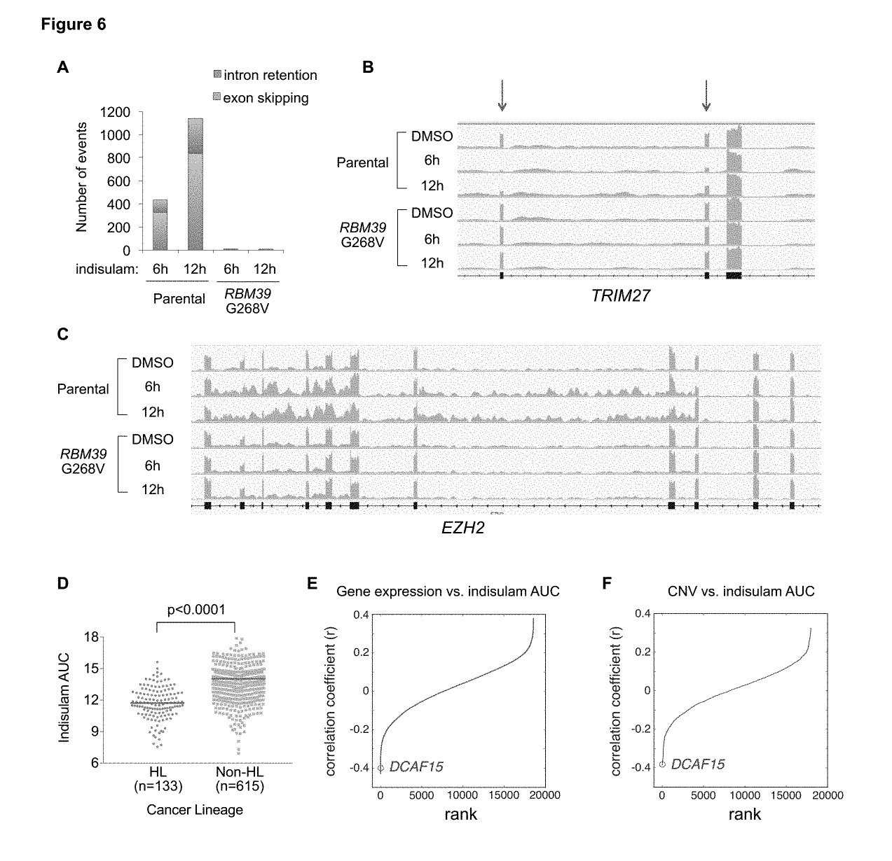

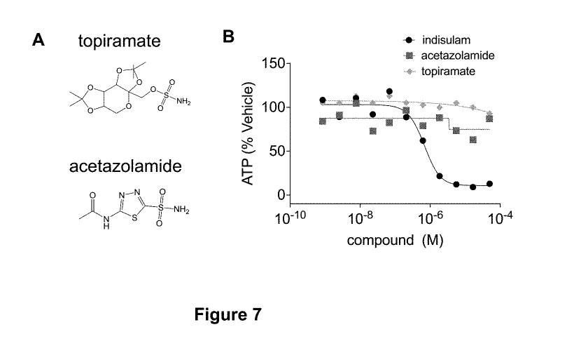

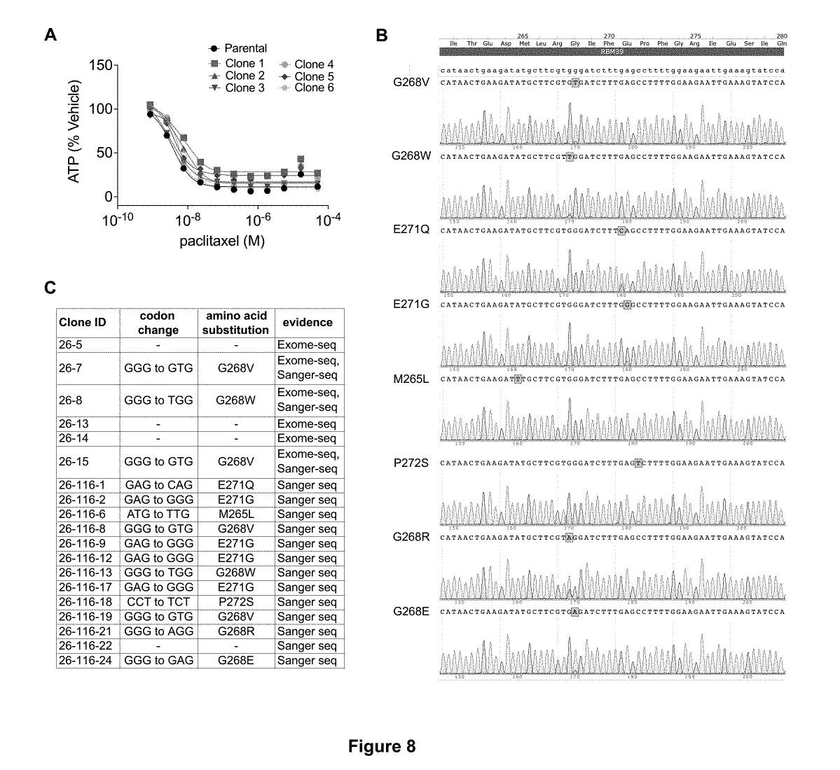

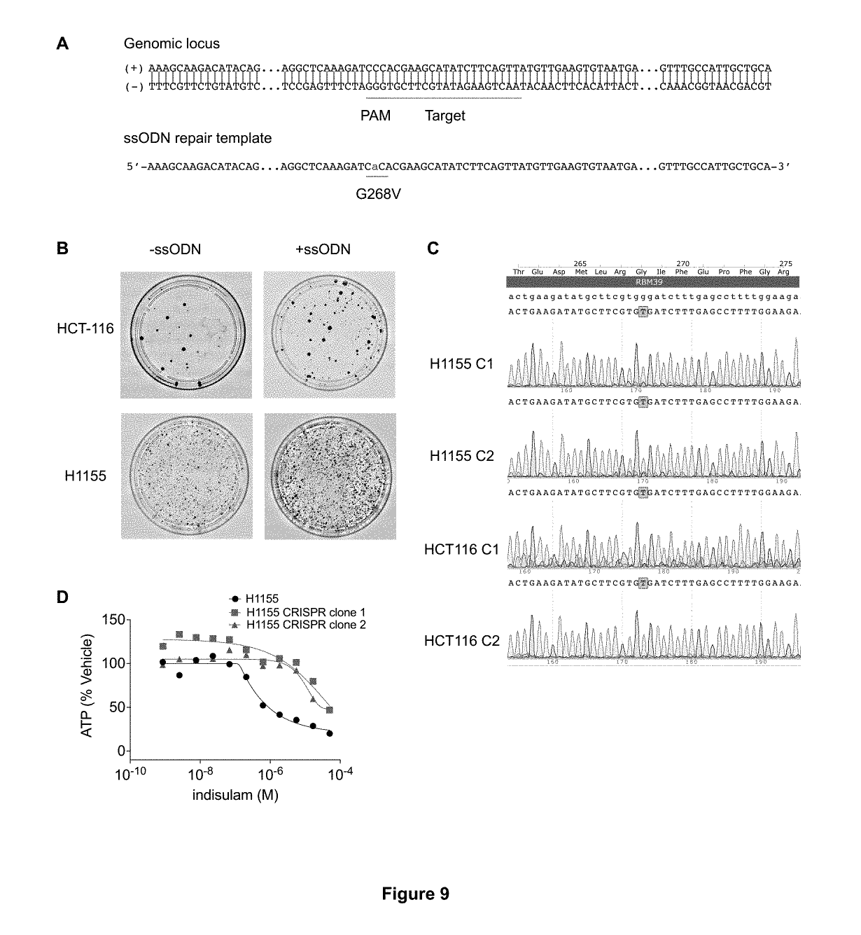

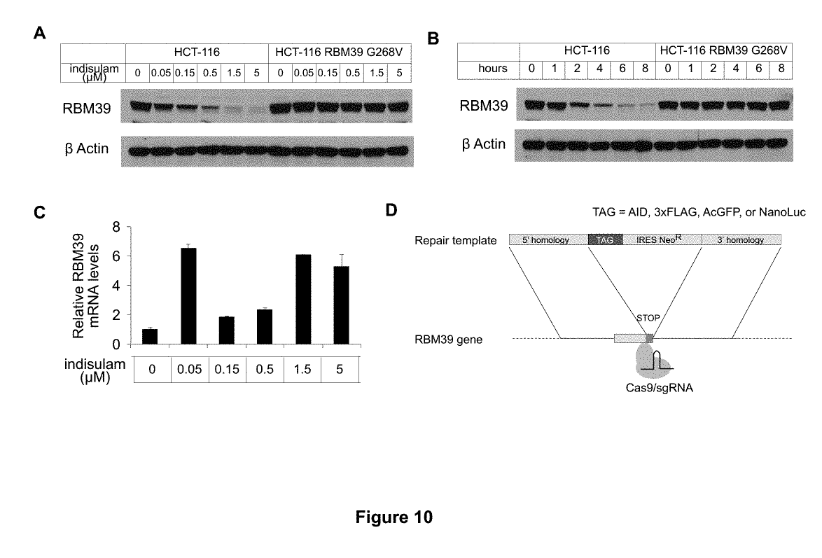

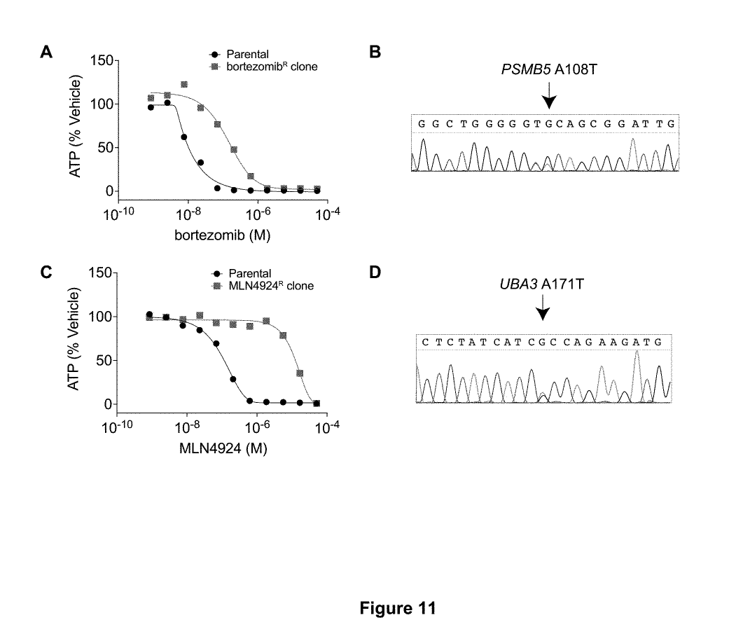

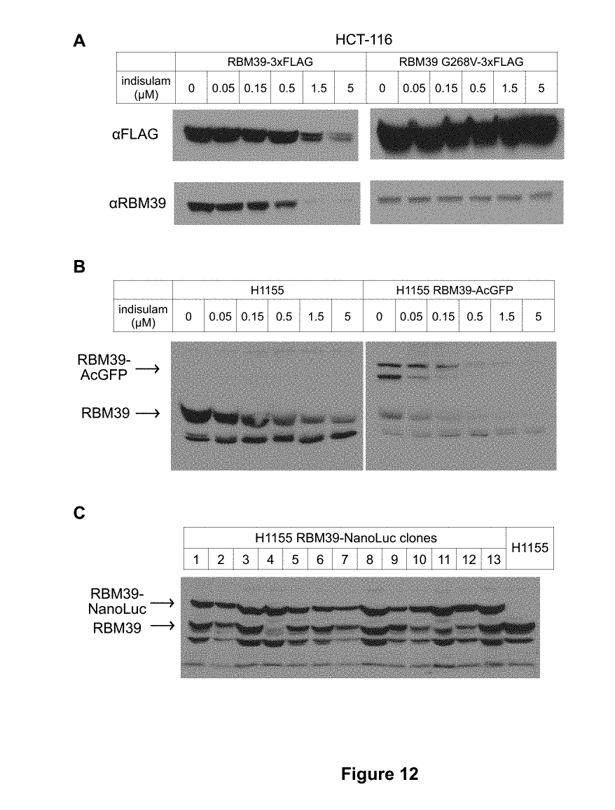

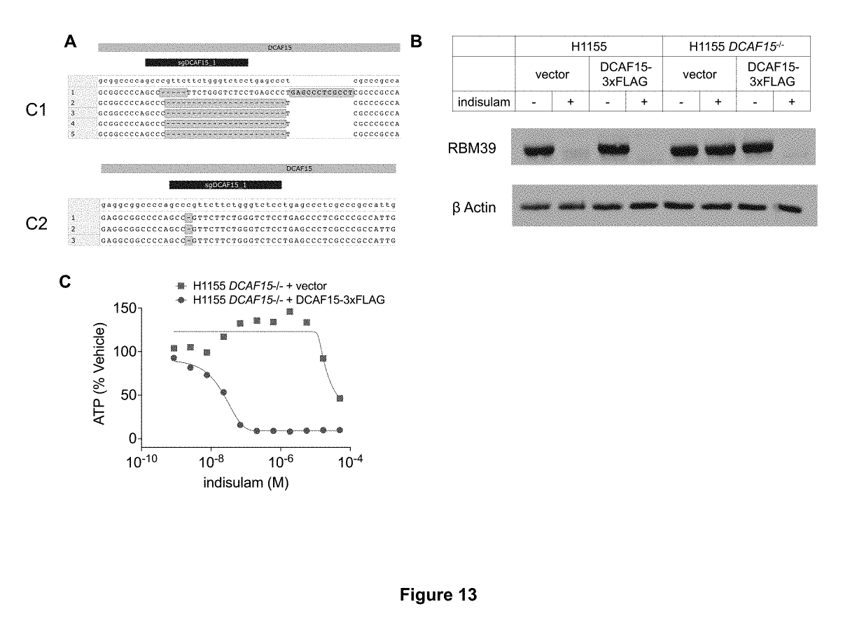

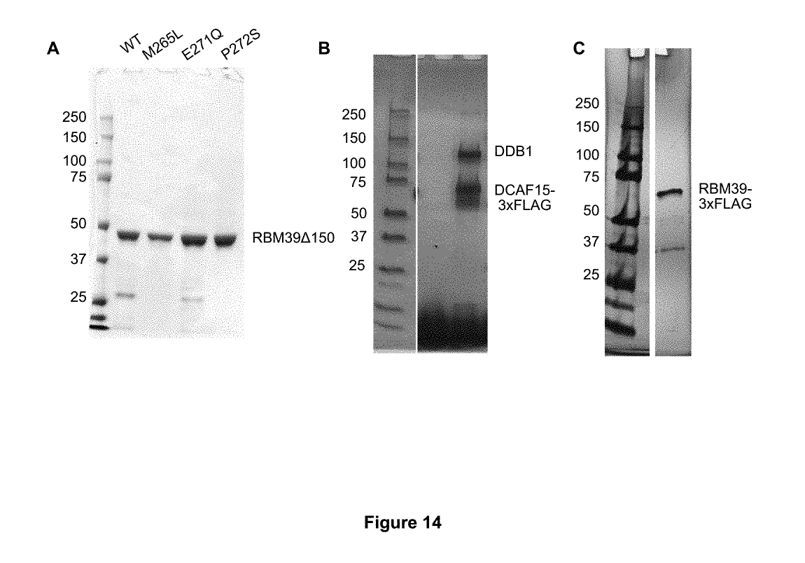

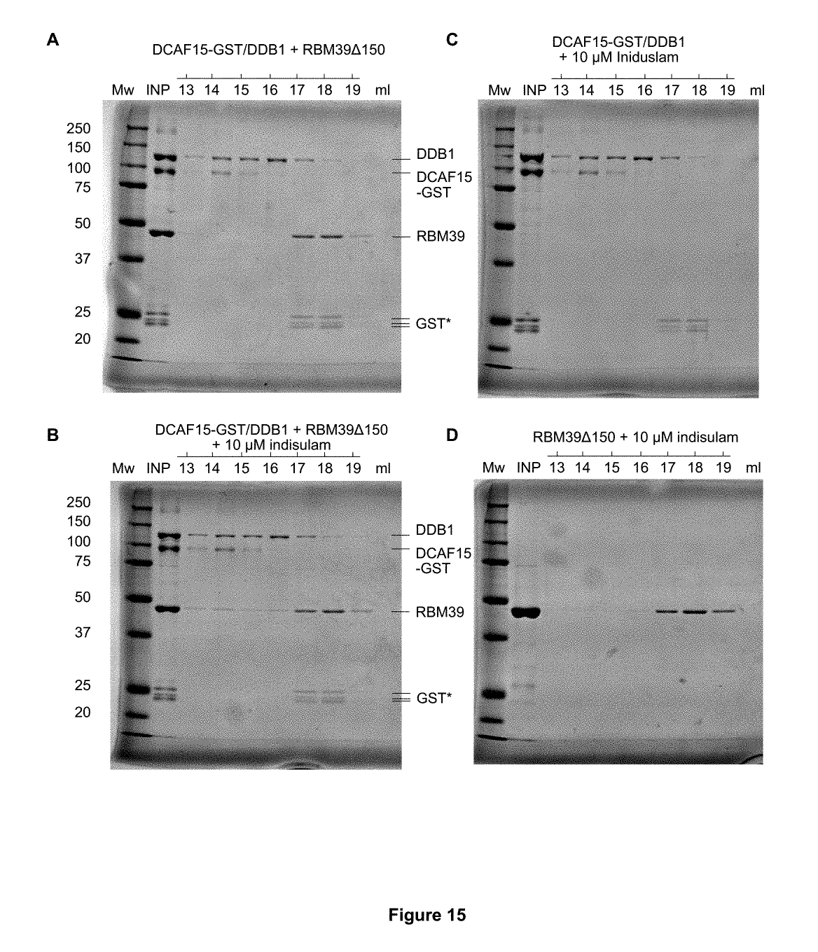

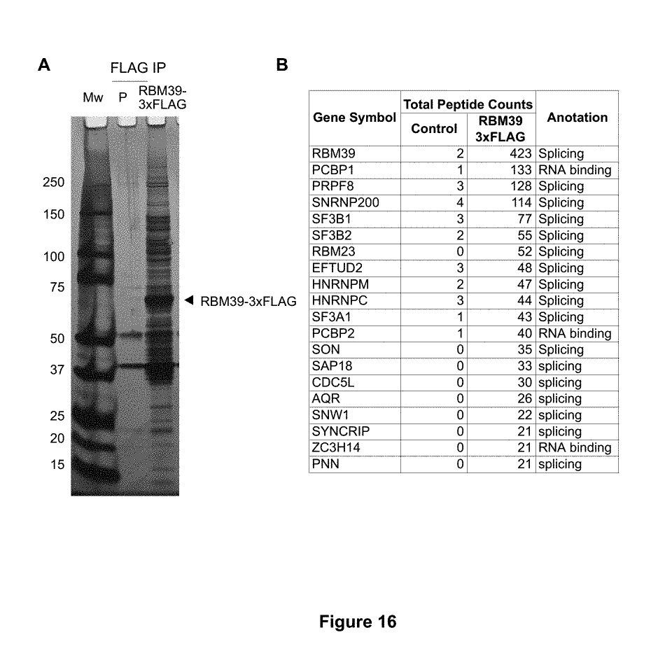

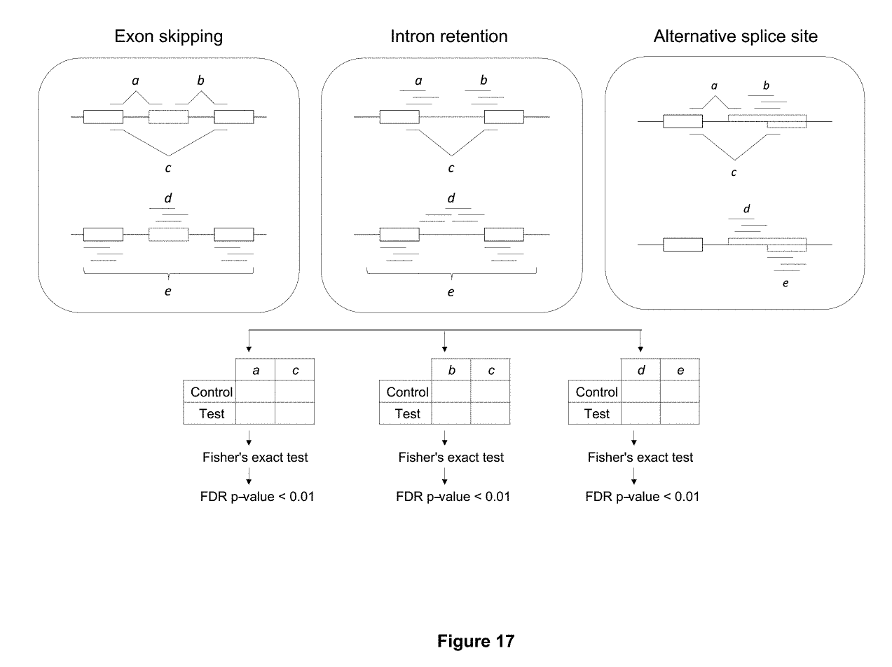

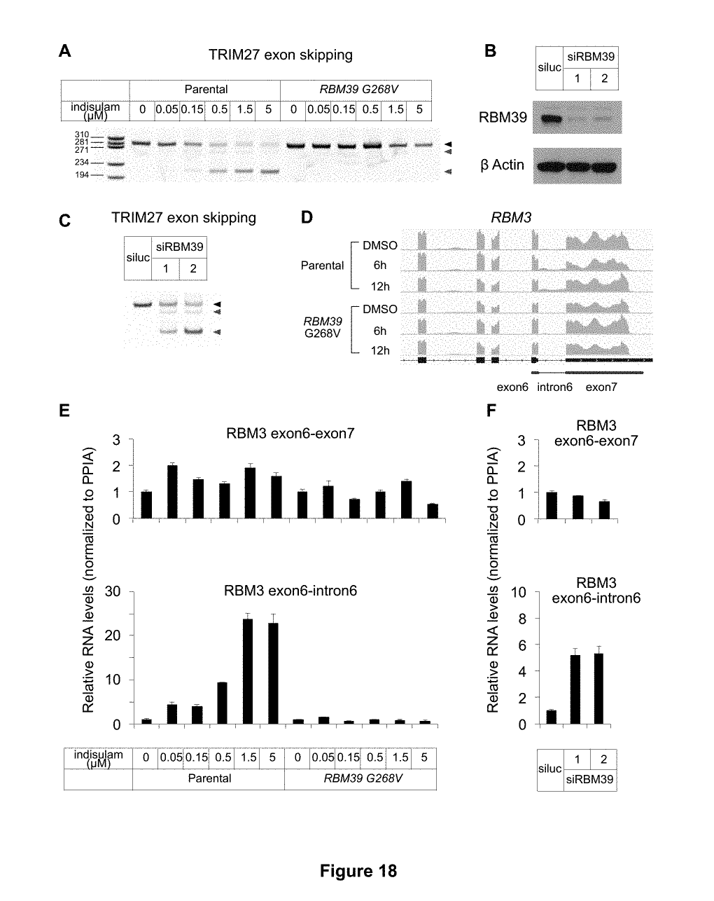

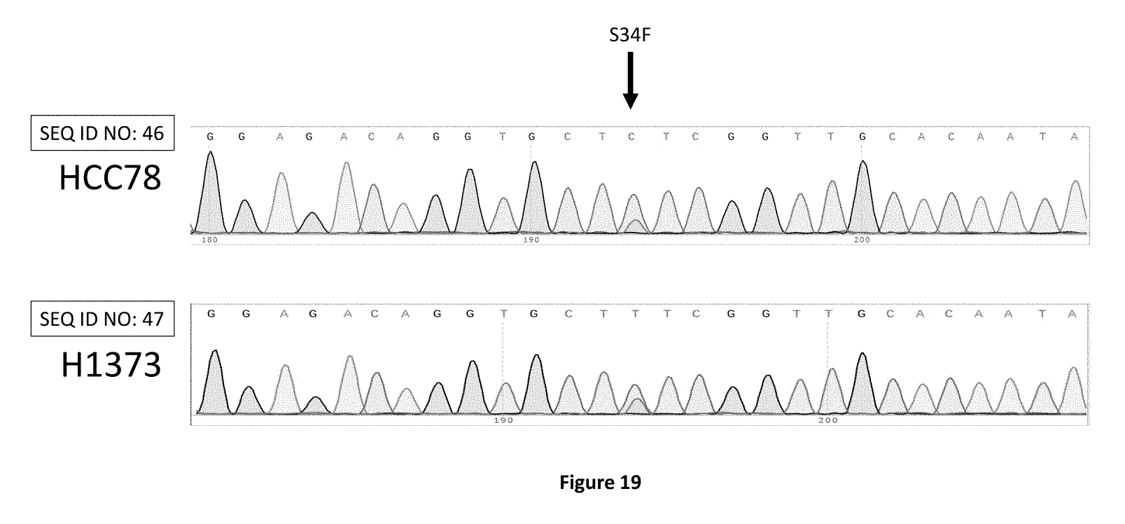

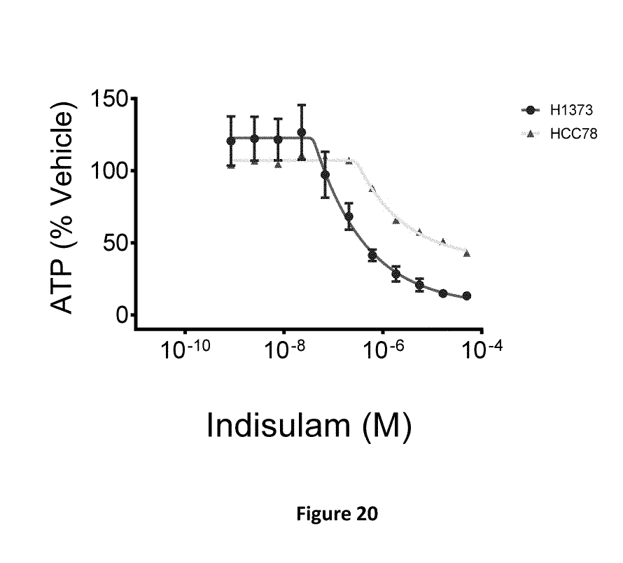

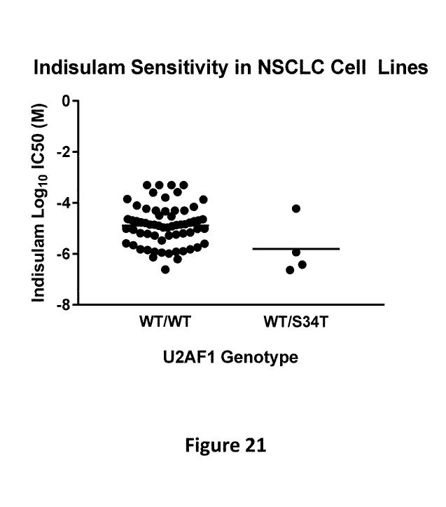

Indisulam (also known as E7070) is an aryl sulfonamide discovered by Eisai pharmaceuticals in a phenotypic screen for small molecules with anti-cancer activity (FIG. 1, panel A) (7, 8). Treatment of human cancer cell lines with indisulam resulted in cell cycle arrest in the G1 phase followed by cell death (7, 8). In a comprehensive analysis of 42 cancer cell lines, indisulam was found to be toxic to a subset of cell lines representing multiple different lineages (9). The selectivity of indisulam was recapitulated in pre-clinical efficacy studies using tumors derived from different human cancer cell lines grafted into immune-deficient mice (9). In multiple phase I and phase II trials involving advanced stage patients with solid tumors, indisulam monotherapy resulted in a modest number of clinical responses and stable disease in 17-36% of patients (10-17). The biologic basis for indisulam sensitivity is unknown and there are currently no biomarkers that can predict which patients might be more likely to respond to indisulam.

Indisulam has been reported to be a potent inhibitor of carbonic anhydrase isoforms (18). We compared the sensitivity of the colorectal cancer cell line, HCT-116, to indisulam and two chemically distinct carbonic anhydrase inhibitors with comparable potency, acetazolamide and topirimate (FIG. 7, panel A). The concentration of indisulam required to reduce the viability of HCT-116 cells by 50% (IC50) was 0.56 μM. In contrast, acetazolamide and topirimate were non-toxic even at concentrations as high as 50 μM (FIG. 7, panel B). We therefore hypothesized that the anti-cancer activity of indisulam might be due to its effects on targets other than carbonic anhydrase.

### Example 1: RBM39 Mutations Cause Indisulam Resistance

A powerful method for discovering the functional targets of anti-cancer small molecules is to identify mutations that render cells resistant to the toxin. Kapoor and colleagues have described a strategy that uses HCT-116 human colorectal carcinoma cells as a tool to discover such compound resistant alleles (19). These cells are defective in mismatch repair and consequently are predisposed to develop resistance through nucleotide substitutions. Previously, we selected for clonal resistance amongst a population of barcoded HCT-116 cells to different anti-tumor toxins (20). Six of these clones were isolated following selection with lethal doses of indisulam. In comparison to the parental HCT-116 cells (IC50=0.56 μM), all six clones were resistant to indisulam (FIG. 1, panel B). Five of the clones were insensitive to indisulam at concentrations as high as 50 and the sixth clone was 17 times less sensitive than the parental HCT-116 cells (IC50=9.3 μM). None of the clones were resistant to the chemically unrelated toxin, paclitaxel, reducing the likelihood that indisulam resistance was the result of a non-specific increase in compound metabolism or efflux (FIG. 8, panel A).

Using exome sequencing data, we identified 634 missense mutations, which were exclusively present in at least one indisulam resistant clone (FIG. 1, panel C). Because mutations that cause indisulam resistance are more likely to recur in multiple clones, we categorized genes by the number of indisulam resistant clones in which they were mutated (FIG. 1, panel C). No gene was mutated in more than three clones, and three genes were mutated in three out of the six clones sequenced. Of these three genes, RBM39 was exceptional because all of the identified mutations affected the same codon. The glycine residue at position 268 of RBM39 was mutated to valine in two independent clones and tryptophan in the third.

To further explore the association of RBM39 mutations with indisulam resistance, we applied parallel selections on 13 clonally derived HCT-116 cell lines, isolated one resistant clone from each selection, and sequenced the RBM39 cDNA in each of them. Within the full complement of 19 indisulam-resistant clones, 15 clones harbored a missense mutation in RBM39. All of these mutations affected one of four amino acid residues located between codons 265 and 272. The glycine residue at position 268 was mutated to valine (n=4), tryptophan (n=2), glutamate (n=1), arginine (n=1). The methionine residue at position 265 was mutated to leucine (n=1); Glutamate residue 271 was mutated to either glycine (n=4) or glutamine (n=1); and proline residue 272 was mutated to serine (n=1) (FIG. 1, panel D; FIG. 8, panels B and C).

The RBM39 protein is composed of an arginine-serine (RS) domain at the N-terminus followed by three predicted RNA recognition motifs (RRM). Mutations in RBM39 that coincide with indisulam resistance clustered in the second RNA recognition motif (RRM2) (FIG. 1, panel D). Using a previously reported NMR structure of the RBM39 RRM2 domain, positions 265, 268, 271, and 272 all map to the same external face of an α helix (FIG. 1, panel E). Based on these observations, we reasoned that the toxic effects of indisulam may require interactions (either protein-protein or protein-compound) involving the surface of this a helix in the RRM2 domain of RBM39.

We next performed experiments to test whether RBM39 mutations specify indisulam resistance. We treated HCT-116 cells that transiently expressed either wild type or mutant RBM39 with indisulam and assessed cell viability (FIG. 1, panel F). Mock-transfected cells and cells transfected with wild type RBM39 were not indisulam resistant. Transient expression of eight different RBM39 mutants, by contrast, conferred resistance to indisulam. These experiments suggest that RBM39 mutations are sufficient for indisulam resistance in HCT-116 cells.

Extending these observations, we used CRISPR/Cas9 technology to introduce the RBM39 G268V mutation into both HCT-116 and H1155 cells, a non-small cell lung cancer cell line sensitive to indisulam. We selected codon 268 for editing because the wild type codon sequence is GGG, encoding a protospacer adjacent motif (PAM). Successful editing of the sequence to GTG encodes for valine and destroys the PAM sequence, preventing the Cas9/sgRNA complex from recutting. We co-transfected plasmids expressing Cas9, an sgRNA designed to target the 19 bp upstream of this PAM sequence, and a single stranded deoxynucleotide (ssODN) encoding the G268V allele (FIG. 9, panel A). These conditions increased clonal resistance to indisulam, confirming our hypothesis that the G268V allele confers resistance (FIG. 9, panel B). Two independent clones contained the expected G268V mutation (FIG. 9, panel C) and were 60- and 80-fold less sensitive to indisulam (IC50=38.9 μM and 52.2 μM) than the parental HCT-116 cells (FIG. 1, panel G). Similarly, G268V editing in H1155 cells also resulted in indisulam resistance (FIG. 9, panel D). On the basis of these data, we conclude that RBM39 mutations in the RRM2 domain are sufficient to confer resistance to indisulam.

After confirming that the anti-cancer activity of indisulam in cultured cancer cells acts through RBM39, we tested this hypothesis in vivo. It has previously been shown that intravenous (IV) administration of 25 mg/kg indisulam for 8 days leads to regression of tumors derived from HCT-116 cells subcutaneously grafted into immune deficient mice (9). Using the same dosing schedule, we compared the ability of indisulam to induce the regression of tumors derived either from parental HCT-116 cells or cells expressing the RBM39 G268V allele (FIG. 1, panel H). We found that indisulam treatment leads to complete regression of tumors derived from parental HCT-116 cells. By contrast, indisulam-treated tumors derived from RBM39 G268V-expressing cells grew at a rate indistinguishable from that of the control group treated with vehicle. We conclude that the in vivo anti-tumor activity of indisulam is attributable to its effects on RBM39.

### Example 2: Indisulam Toxicity Requires RBM39 Proteasomal Degradation

To investigate the mechanism by which RBM39 mutations confer resistance, we used western blotting to analyze RBM39 protein in indisulam-treated HCT-116 cells. The amount of RBM39 protein was reduced in HCT-116 cells in an indisulam dose dependent manner and this effect was evident as early as two hours after treatment (FIG. 10, panels A and B). The reduction in RBM39 protein was post-translational since we observed no dose dependent decrease in RBM39 mRNA after indisulam treatment (FIG. 10, panel C). Notably, indisulam had no effect on RBM39 protein levels in resistant cells harboring indisulam-resistant mutations (FIG. 2, panel A; FIG. 10, panels A and B).

To ascertain whether RBM39 degradation is sufficient for cell death, we adopted the auxin-inducible degron (AID) system to selectively degrade RBM39 (21). Auxin (also known as 3-indoloacetic acid or IAA) is a plant hormone that promotes the ubiquitination and proteasomal degradation of proteins containing an AID domain by recruiting that domain to the plant E3 ubiquitin ligase receptor, TIR1 (21). In human cells that ectopically express TIR1, IAA has been used to degrade AID-tagged proteins (22). Therefore, we used CRISPR/Cas9 to knock-in a sequence encoding the AID domain at the 3-prime end of the RBM39 gene (FIG. 10, panel D). As predicted, IAA treatment led to a decrease in RBM39 levels only in cells that express both RBM39-AID and TIR1 (FIG. 2, panel B). Furthermore, the consequence of RBM39 degradation in these cells was a loss in cell viability (FIG. 2, panel C). Collectively, these experiments provide two independent lines of evidence that indisulam is toxic to cells by degrading RBM39: (i) targeted degradation of RBM39 through an alternative mechanism leads to a loss in cell viability; and (ii) mutations in RBM39 that prevent degradation lead to indisulam resistance.

To determine the mechanism by which indisulam leads to RBM39 degradation, we first examined whether RBM39 degradation requires the proteasome. The reduction in RBM39 protein could be blocked by bortezomib, which inhibits the catalytic 20S proteasome complex by directly targeting the PSMB5 proteasome subunit. The PSMB5 A108T mutation is known to render cells relatively resistant to proteasome inhibition by bortezomib, thereby providing a tool with which to validate that any observed effect of bortezomib is indeed on-target (FIG. 11, panels A and B) (19). We studied how bortezomib influenced indisulam dependent loss of RBM39 in parental and in PSMB5 A108T cells (FIG. 2, panel D). In parental cells, the addition of 10 nM of bortezomib blocked the degradation of RBM39 in response to indisulam. In the PSMB5 A108T cell line, comparable rescue of RBM39 levels required a 10-fold higher level of bortezomib. This difference confirms that the rescue of RBM39 levels by bortezomib is the consequence of targeting PSMB5 in the proteasome. Together, these results suggest that the reduction in RBM39 levels following indisulam treatment depends on the activity of the proteasome.

### Example 3: Indisulam Recruits RBM39 to CUL4-DCAF15

The canonical pathway for proteasome mediated degradation requires post-translational modification of the substrate with poly-ubiquitin. In many cases, ubiquitin is added to substrates by Cullin RING Ligases (CRL), which are multi-subunit ubiquitin ligases (23). CRLs are modular complexes that contain a common catalytic core but assemble with a diverse set of receptors. These receptors recruit specific substrates to the CRL catalytic complex. The catalytic activity of all CRLs requires activation through the post-translational modification of the Cullin with NEDD8, a ubiquitin-like peptide (24). The activation of NEDD8 is catalyzed by Neddylation activating E1 enzyme (NAE), which is a heterodimeric complex consisting of regulatory (APPBP1) and catalytic (UBA3) subunits (25). MLN4924 is a small molecule inhibitor of UBA3 and is toxic to cells because it blocks neddylation of CRLs (25). Accordingly, the UBA3 A171T mutation renders cells resistant to the toxic effects of MLN4924, providing a tool to validate any observed effect of MLN4924 as on-target (FIG. 11, C and D) (26). The addition of MLN4924 blocked the degradation of RBM39 in response to indisulam in HCT-116 cells (FIG. 2, panel E). However, we did not observe this effect in cells harboring a UBA3 A171T mutation, thus confirming that the observed rescue in RBM39 levels is indeed due to MLN4924's action on UBA3. Based on these observations, we concluded that indisulam may require a CRL complex to degrade RBM39.

We next used a combination of biochemical purification and proteomics to identify the specific CRL complex that associates with RBM39 after indisulam treatment. We first generated reagents that could be used to purify and enrich endogenous RBM39 complexes. Specifically, we used CRISPR/Cas9 engineering to introduce a sequence encoding a C-terminal 3xFLAG tag into endogenous RBM39. Importantly, RBM39-3xFLAG protein is fully susceptible to indisulam triggered degradation (FIG. 12, panel A). We purified RBM39-3xFLAG complexes with Anti-FLAG beads from lysate isolated from cells treated with either DMSO or indisulam. To identify the protein components of these complexes, we used liquid chromatography and mass spectrometry to analyze tryptic peptides collected from the complexes (a procedure commonly referred to as shotgun mass spectrometry). After common contaminants were removed, 739 and 688 proteins were identified in the vehicle- and indisulam-treated RBM39-3xFLAG complexes, respectively (table 1). To identify proteins that specifically associate with RBM39 in the presence of indisulam, we focused our attention on 15 proteins for which the number of spectral counts was greater than 3 in indisulam-treated samples and 0 in the DMSO-treated control sample. We likewise identified 3 additional proteins for which the number of spectral counts was at least 10 fold higher in the indisulam-treated sample (table 1). Among these 18 proteins were components of a known CRL complex: Cullin 4A and Cullin 4B (hereafter collectively referred to as CUL4), DDB1, DDA1, and DCAF15. DDB1 and DDA1 are adaptors common to many CUL4 CRLs, whereas DCAF15 (DDB1 CUL4 Associated Factor 15) is a substrate-specific receptor (FIG. 2, panel F) (27). Of note, we did not identify RBX1, which is an essential component of CRLs required for recruiting E2 ubiquitin conjugating enzyme. This is likely either the consequence of fewer representative peptides given its small size (108 amino acids) or its low affinity for the Cullin scaffold (28). Based on these results, we concluded that indisulam treatment induces recruitment of RBM39 to a CUL4, DDB1, DDA1, DCAF15 CRL complex (CUL4-DCAF15).

We proceeded to systematically test whether the components of CUL4-DCAF15 were individually essential for indisulam-dependent degradation of RBM39. First, we tested the role of different Cullins by expressing truncated forms of the test proteins. Truncated Cullins can still associate with Cullin-specific adaptors but are catalytically inactive, and therefore function in a dominant negative (DN) manner (23, 29, 30). Expression of CUL1-DN, CUL2-DN, CUL3-DN, and CUL5-DN had no effect on indisulam-dependent RBM39 degradation. CUL4A-DN and CUL4B-DN expression, by contrast, inhibited RBM39 degradation (FIG. 3, panel A). These observations provide evidence that a CUL4 complex is required for RBM39 degradation.

To test whether other components of the complex—DDB1, DDA1 or DCAF15—might also be essential for RBM39 degradation, we studied a H1155 cell line in which we used CRISPR/Cas9 engineering to introduce a sequence encoding AcGFP into the C-terminus of RBM39. Like RBM39, RBM39-AcGFP is degraded after indisulam treatment (FIG. 12, panel B); thus we were able to evaluate RBM39 degradation in individual cells by fluorescence-activated cell sorting (FACS). We transfected these cells with a plasmid expressing both Cas9 and sgRNAs designed to target DDB1, DDA1, or DCAF15. For each gene, we tested two independent sgRNA sequences. Following transfection, we analyzed vehicle- or indisulam-treated cells for green fluorescent protein (GFP) by FACS (FIG. 3, panel B). A subset of cells failed to degrade RBM39-AcGFP in response to indisulam when either of the two guide RNAs targeting DDB1, DDA1, or DCAF15 was transfected.

Following CRISPR editing with DCAF15 sgRNA, we isolated two clones in which DCAF15 was inactivated (FIG. 13, panel A). DCAF15−/− cells failed to degrade RBM39 in response to indisulam and were also insensitive to indisulam even at concentrations as high as 50 μM (FIG. 3, panel C; FIG. 13, panel B). To confirm that indisulam resistance and the impaired degradation of RBM39 were due to loss of DCAF15, we stably expressed a tagged form of DCAF15 in DCAF15−/− clones. Expression of tagged DCAF15 restored indisulam dependent RBM39 degradation as well as indisulam sensitivity (FIG. 13, B and C). Furthermore, H1155 cells engineered to express higher than normal levels of DCAF15 were more sensitive to indisulam than mock treated cells (FIG. 3, panel D). It is notable that the basal levels of RBM39 were unchanged regardless of alterations in DCAF15 expression, suggesting that RBM39 is not an endogenous substrate for DCAF15 (FIG. 13, panel B).

We reasoned that mutant RBM39 may not be degraded because it is unable to associate with the CUL4-DCAF15 complex in the presence of indisulam. To test this hypothesis, we immunopurified RBM39 with Anti-FLAG beads using lysate derived from parental cells, RBM39-3xFLAG cells, or RBM39 G268V-3xFLAG cells. Purified complexes were analyzed by western blotting using antibodies to DDA1, DDB1, CUL4A, and CUL4B (FIG. 3, panel E). RBM39-3xFLAG co-purified with DDA1, DDB1, CUL4A, and CUL4B in cells treated with indisulam but not DMSO. RBM39 G268V, by contrast, was not recruited to the CUL4 complex even in the presence of indisulam. This experiment provided evidence that recruitment to CUL4-DCAF15 is required for RBM39 degradation and explains how RBM39 mutations resist degradation.

To directly test whether these proteins influence RBM39 ubiquitination, we co-transfected cells with plasmids encoding DCAF15, 6x-His tagged ubiquitin, and either wild type or mutant RBM39. We analyzed RBM39 protein by western blotting following purification of lysate by nickel chromatography, which enriches for proteins modified with the expressed His-ubiquitin (FIG. 3, panel F). We observed increased RBM39 ubiquitination following indisulam treatment when DCAF15 was co-expressed. By contrast, RBM39 G268V was not ubiquitinated following indisulam treatment. From these results, we conclude that indisulam-triggered recruitment to the CUL4-DCAF15 complex leads to RBM39 poly-ubiquitination, degradation by the proteasome, and cell death. The indisulam resistant mutations, which lie on the surface of an α helix in the RRM2 domain of RBM39, block the recruitment of the CUL4-DCAF15 complex. As such, we predict that cells survive because RBM39 does not undergo ubiquitin-proteasome mediated degradation.

### Example 4: RBM39-DCAF15 is the Direct Indisulam Target

We next conducted a set of experiments to determine the mechanism by which indisulam recruits RBM39 to the CUL4-DCAF15 complex. First, we reconstituted formation of the RBM39-CUL4-DCAF15 complex in vitro (FIG. 4, panel A). Lysates derived from parental cells, cells expressing RBM39-3xFLAG, or RBM39-3xFLAG G268V were supplemented with increasing concentrations of indisulam and then immuno-purified with Anti-FLAG. DDA1, DDB1, and CUL4A co-purified with RBM39-3xFLAG in an indisulam dose-dependent manner. By contrast, RBM39-3xFLAG G268V did not co-purify with the CUL4-DCAF15 complex even in the presence of 10 μM of indisulam. These results demonstrate that indisulam can recruit RBM39 to CRL-DCAF15 in vitro.

We used a recombinant system to determine if one of the proteins in the CUL4-DCAF15 complex might interact with RBM39 in an indisulam-dependent manner. Using insect cells, we expressed and purified recombinant RBM39 containing the RRM1, RRM2, and RRM3 domains, hereafter referred to as RBM39Δ150 (FIG. 14, panel A). We then measured the amount of RBM39Δ150 that co-purified with each of these proteins in the presence of either vehicle or indisulam (FIG. 4, panel B). DCAF15 alone, but not DDB1, CUL4A, or CUL4B, co-purified with RBM39Δ150 in the presence of indisulam. Of note, DCAF15 co-purified with DDB1, consistent with DDB1's known function as an adaptor that binds both CUL4 and DCAF receptors (FIG. 14, panel B) (31). Since DDB1 alone failed to bind RBM39 in the presence of indisulam, we conclude that DCAF15 provides the critical interactions for indisulam and RBM39. Therefore, this experiment suggests that recruitment of RBM39 to CUL4-DCAF15 is mediated by an interaction between DCAF15 and RBM39.

We next compared the ability of indisulam to recruit either wild type or mutant RBM39 to DCAF15 (FIG. 4, panel C). Evidence of the RBM39Δ150-DCAF15 complex was apparent in the presence of 0.10 μM indisulam. A 100-fold higher concentration of indisulam was required to recruit RBM39Δ150 P272S to DCAF15. Finally, at concentrations as high as 10 μM, indisulam failed to recruit the M295L or E271Q variants of RBM39Δ150 to DCAF15.

We then used liquid chromatography and mass spectrometry to analyze the levels of indisulam in the RBM39Δ150-DCAF15 complex (FIG. 4, panel D). We incubated 10 μM indisulam with purified DCAF15 alone, full length RBM39 alone (FIG. 14, panel C), or a mixture of DCAF15 and RBM39Δ150. Following organic extractions, the amount of indisulam was determined in each fraction. The amount of indisulam corresponded directly to the amount of RBM39Δ150 in the complex: wild type RBM39Δ150 had the highest levels of indisulam, P272S had lower levels of indisulam, and no indisulam was detected in the presence of the M265L or E271Q variants of RBM39Δ150. Moreover, no indisulam was identified in samples containing either DCAF15 or RBM39 alone. These findings suggest that indisulam binds to a complex containing both DCAF15 and RBM39.

To confirm that this is a soluble complex, we also analyzed recombinant DDB1:DCAF15-GST (expressed and purified from Sf9 cells) and RBM39Δ150 using size exclusion chromatography (FIG. 15). We incubated 0.62 μM of DDB1:DCAF15-GST and 1.76 μM RBM39Δ150 in the presence of DMSO or 10 μM indisulam, performed size exclusion chromatography, and then analyzed eluting fractions by SDS-PAGE followed by Coomassie staining. In the DMSO-treated sample, DDB1:DCAF15-GST elutes as two peaks representing the heterodimer and free DDB1, and RBM39Δ150 migrates as a single peak (FIG. 15, panel A). In the presence of indisulam, a subset of RBM39Δ150 co-elutes with the DDB1:DCAF15-GST peak (FIG. 15, panel B). The indisulam-dependent shift in the elution profile only occurs when both preparations are incubated together. Indisulam has no impact on the elution profile of either RBM39Δ150 or DDB1:DCAF15-GST alone (FIG. 15, C and D). These results provide further evidence that indisulam promotes a direct interaction between DCAF15 and RBM39. These experiments collectively support a model in which indisulam mediates interaction between DCAF15 and RBM39. DCAF15 further recruits CUL4-DDB1-DDA1 E3 ubiquitin ligase complex, which triggers ubiquitination and degradation of RBM39 (FIG. 4, panel E). Consistent with this model, mutations in RBM39 that prevent its recruitment to DCAF15 in vitro also prevent ubiquitination and degradation in vivo. Accordingly, these mutations confer resistance to the toxic effects of indisulam because RBM39 is not degraded.

### Example 5: Other Clinically Tested Sulfonamides Exhibit the Same Mechanism of Action as Indisulam

Like indisulam, tasisulam and chloroquinaxoline sulfonamide (CQS) are aryl sulfonamides with anti-cancer activity that have been used in clinical trials for the treatment of solid tumors (FIG. 5, panel A) (32-37). Based on their structural similarity to indisulam, we hypothesized that tasisulam and CQS might also recruit RBM39 to DCAF15. Indeed, both tasisulam and CQS recruit RBM39Δ150 to DCAF15 in a dose-dependent manner (FIG. 5, panel B); however, indisulam-resistant mutants of RBM39 were not effectively recruited to DCAF15 in the presence of these compounds. We next tested whether tasisulam or CQS might trigger RBM39 degradation in cells. To test this hypothesis, we treated H1155 cells in which we used CRISPR/Cas9 to introduce sequence encoding NanoLuc luciferse into the C-terminus of RBM39 (RBM39-Nluc) (FIG. 12, panel C). As expected, indisulam caused a dose-dependent decrease in RBM39-Nluc activity (IC50=0.36 μM) (FIG. 5, panel C). Both tasisulam and CQS also caused a dose-dependent decrease in RBM39-Nluc activity, albeit less potently than indisulam (IC50=5.6 μM and IC50=2.4 respectively). Not surprisingly, CQS and tasisulam were also toxic to HCT-116 cells (IC50=7.1 μM and IC50=6.5 respectively), and a cell line carrying the RBM39 G268V mutation was insensitive to the two toxins even at concentrations as high as 50 μM (FIG. 5, D and E). These observations suggest that indisulam, tasisulam and CQS share the same mechanism of action.

### Example 6: Indisulam-Induced Degradation of RBM39 Leads to Splicing Defects

RBM39 is related to U2AF2 (U2 small nuclear RNA auxiliary factor 2), a component of the U2 snRNP complex of the spliceosome. Purification of RBM39-3xFLAG complex followed by mass spectrometry analysis revealed association of RBM39 with numerous splicing factors (FIG. 16 and table 2). RBM39 co-localizes with SRSF2, a canonical splicing factor, to nuclear speckles, domains enriched in pre-mRNA splicing proteins (38). We hypothesized that RBM39 degradation by indisulam would lead to splicing defects. To examine the consequence of RBM39 inactivation on RNA splicing, we collected total RNA from either HCT-116 cells or HCT-116 cells carrying the RBM39 G268V mutation, both of which were treated in parallel with 2 μM indisulam for 6 or 12 hours. After depletion of ribosomal RNA, we performed RNA sequencing. We evaluated the resulting sequencing reads for six major classes of splicing changes (FIG. 17) and found exon skipping and intron retention to be the predominant form of splicing defects after indisulam treatment (FIG. 6, panel A). In parental HCT-116 cells, at 6 and 12 hours of treatment, 327 and 837 exon skipping events were detected (FIG. 6, panels A, B). Only 3 exon skipping events were found in HCT-116 cells harboring a RBM39 G268V mutation. These data provide evidence that the splicing defects are indeed due to RBM39 degradation rather than being an off-target effect. A similar pattern was observed with respect to intron retention events (FIGS. 6, A and C). At 6 and 12 hours after indisulam treatment, 107 and 303 introns, respectively, were retained in parental HCT-116 cells. In contrast, no intron retention event was found in cells expressing the RBM39 G268V allele. Using RT-PCR assays, we observed indisulam dose-dependent exon skipping in TRIM27 mRNA and intron retention in RBM3 mRNA (FIG. 18, panels A, D, and E). Furthermore, knockdown of RBM39 by two independent siRNAs recapitulated the effects of indisulam on the splicing of TRIM27 and RBM3 (FIG. 18, panels B, C, and F). These results support the hypothesis that indisulam toxicity results from the disruption of RBM39-dependent mRNA splicing, leading to exon skipping and intron retention.

### Example 7: DCAF15 Levels Correlate with Indisulam Sensitivity

The mechanism of action of indisulam suggests that cancer cells expressing higher levels of DCAF15 may exhibit hypersensitivity to indisulam. This hypothesis is supported by experiments in an isogenic system, which demonstrate that DCAF15 is not only essential for indisulam toxicity, but that ectopic expression of DCAF15 increases the potency of indisulam (FIG. 3, C and D). We therefore asked whether DCAF15 expression or copy number alterations might correlate with indisulam sensitivity among a collection of genetically heterogeneous cancer cell lines. The Cancer Target Discovery (CTD2) Network has provided a resource that profiled 758 cancer cell lines representing different lineages and genotypes for compound sensitivity, copy number variation, and gene expression (39-41). The responses to indisulam were reported as Area-Under-the-Curve (AUC) values that reflect the degree of sensitivity to indisulam. Investigators from the CTD2 found that sensitivity to indisulam was significantly enriched amongst cancer cell lines derived from the hematopoietic and lymphoid cell (HL) lineage (FIG. 6, panel D) (39). In addition, they reported that the basal mRNA expression or copy number of DCAF15 is inversely correlated with indisulam AUC in 133 HL cancer cell lines (41). We reproduced these findings by reanalyzing the CTD2 data. Re-analysis of correlation of mRNA expression with indisulam AUC revealed a distribution of correlation coefficients ranging from 0.380 (high expression correlates with indisulam resistance) to −0.430 (high expression correlates with indisulam sensitivity) (table 3). DCAF15 is a notable outlier in the latter group (z=−3.46) ranking 4^{th} out of 18,541 transcripts with r=−0.400 (FIG. 6, panel E). An analogous analysis that correlates copy number variants (CNV) in the same 133 HL cancer cell lines with indisulam sensitivity revealed correlations ranging from −0.387 to 0.327 (table 4). Of the 17,961 genes with copy number data, DCAF15 was the 5th highest rank gene (z=−3.43) in which copy number correlated with indisulam sensitivity (r=−0.381) (FIG. 6, panel F). Among HL cancer cell lines representing different genotypes, DCAF15 expression and copy number significantly correlate with indisulam sensitivity (p<0.00001 random permutation test). These correlations are consistent with the mechanism of action of indisulam described here.

### Example 8: Splicing Factor Mutations as Biomarker for Cancer Diagnosis

HCC78 and H1373 non-small cell lung cancer cell lines harbor a heterozygous mutations (C to T) in U2AF1 leading to a Serine to Phenylalanine mutation at position 34. FIG. 19 depicts Sanger sequencing of a RT-PCR product from both cell lines. Cancer cell lines harboring a mutation in U2AF1 are sensitive to Indisulam. The viability of two different lung cancer cell lines was measured in a 3 day viability assay using a range of Indisulam doses and shown in FIG. 20. Thus, mutations in splicing factor U2AF1 can be used as a biomarker for treating cancer, in a DCAF15 independent manner or together with the DCAF15 biomarker.

### Example 9: Add New Data

An IC50 assay (FIG. 21) was conducted to demonstrate that cancer cell lines that express U2AF1 S34F mutations are significantly more sensitive to indisulam than cell lines that express U2AF1 wild type. In this assay, sixty-six non-small cell lung cancer cell lines with wild type U2AF1 and four non-small cell lung cancer cell lines that express U2AF1 S34F mutations were analyzed for their sensitivity to indisulam. Cells were plated at ˜15% confluence in a media comprising 5% concentration of fetal bovine serum supplemented with RPMI, which included 2 mM L-Glutamine in a 96 well dish. Indisulam was then added in a dose response manner from 0 to 50 μM (biological duplicates). Following three days of incubation in a 5% CO₂ incubator, the total ATP in each well was quantified using Cell Titer Glo (Promega). The ATP levels were plotted for each concentration and Graphpad was used to generate a curve (four parameter) from which the concentration required to reduce ATP by 50% (IC50) was determined. The solid lines represent the medians. The IC50 assay shows non-small cell lung cancer cell lines with U2AF1 mutations are significantly (p=0.01, one tailed t-test) more sensitive to indisulam than cell lines that express U2AF1 wild type.

Raw data of the IC50 assay results are shown in Table 5 below:

TABLE 5

| Cell Line | U2AF1 Mutation | Indisulam IC50 | Indisulam IC50 (log 10) |
|---|---|---|---|
| Name | Status | (M) | (M) |
| H1373 | S34F | 2.30E−07 | −6.638083381 |
| H1155 | WT | 2.416E−07 | −6.61690307 |
| LC-2/AD | S34F | 0.000000376 | −6.424812155 |
| H2106 | WT | 6.16E−07 | −6.210489796 |
| HCC4006 | WT | 7.349E−07 | −6.133771753 |
| H838 | WT | 1.03E−06 | −5.988429556 |
| H292 | WT | 0.000001116 | −5.952335805 |
| HCC78 | S34F | 0.000001151 | −5.938924676 |
| H2452 | WT | 1.21E−06 | −5.919012953 |
| H522 | WT | 1.22E−06 | −5.914709422 |
| H2228 | WT | 1.26E−06 | −5.898940645 |
| H1993 | WT | 0.000001423 | −5.8467951 |
| Calu-6 | WT | 0.000001508 | −5.821598658 |
| H1975 | WT | 1.51E−06 | −5.821598658 |
| H720 | WT | 1.79E−06 | −5.747146969 |
| H920 | WT | 0.00000217 | −5.663540266 |
| H1355 | WT | 0.000002497 | −5.602581458 |
| H2073 | WT | 2.57E−06 | −5.590066877 |
| H1703 | WT | 3.33E−06 | −5.477425367 |
| H1693 | WT | 5.17E−06 | −5.286425462 |
| H820 | WT | 0.000005171 | −5.286425462 |
| HCC2279 | WT | 0.000005683 | −5.245422344 |
| HOP62 | WT | 0.000006081 | −5.216024997 |
| HCC15 | WT | 0.000006317 | −5.199489123 |
| HCC2018 | WT | 0.000006483 | −5.188223978 |
| HCC366 | WT | 0.000006914 | −5.160270625 |
| HCC2814 | WT | 0.000008717 | −5.059632954 |
| Calu-1 | WT | 0.000008971 | −5.047159143 |
| HCC95 | WT | 0.000009498 | −5.022367835 |
| H1819 | WT | 9.60E−06 | −5.017774008 |
| H2887 | WT | 9.63E−06 | −5.016328617 |
| WI38 | WT | 0.00001001 | −4.999565923 |
| H125 | WT | 0.00001078 | −4.967381239 |
| H1395 | WT | 1.09E−05 | −4.962175249 |
| H2882 | WT | 1.22E−05 | −4.915066425 |
| HCC4017 | WT | 1.26E−05 | −4.898596649 |
| H2347 | WT | 0.00001348 | −4.870310108 |
| H650 | WT | 0.00001364 | −4.86518563 |
| A427 | WT | 0.00001394 | −4.855737226 |
| H1437 | WT | 0.00001403 | −4.852942329 |
| H2444 | WT | 0.00001408 | −4.851397345 |
| H650 | WT | 0.00001448 | −4.839231438 |
| H1770 | WT | 1.62E−05 | −4.790216985 |
| H1648 | WT | 0.00001683 | −4.773915884 |
| H2009 | WT | 1.74E−05 | −4.760200182 |
| H2122 | WT | 1.88E−05 | −4.72607322 |
| H1581 | WT | 1.92E−05 | −4.715794932 |
| H2172 | WT | 0.00002049 | −4.688458042 |
| H2126 | WT | 0.00002052 | −4.687822644 |
| HCC1438 | WT | 0.00002274 | −4.64320954 |
| HCC44 | WT | 2.30E−05 | −4.637706062 |
| H2170 | WT | 0.00002958 | −4.52900183 |
| H1650 | WT | 0.00003185 | −4.496890563 |
| A549 | WT | 0.00004663 | −4.331334585 |
| Calu-1 | WT | 5.00E−05 | −4.301029996 |
| HCC1359 | WT | 5.00E−05 | −4.301029996 |
| HCC1359 | WT | 5.00E−05 | −4.301029996 |
| H2342 | WT | 0.00005863 | −4.231880106 |
| H441 | S34F | 0.00005948 | −4.22562904 |
| H3122 | WT | 0.00007006 | −4.154529867 |
| H2030 | WT | 0.0000791 | −4.101823517 |
| HCC2450 | WT | 0.0001346 | −3.87095494 |
| H157 | WT | 0.0001405 | −3.852323676 |
| H1944 | WT | 0.0001626 | −3.788879459 |
| H1755 | WT | 0.0002519 | −3.598771833 |
| H1568 | WT | 0.0002653 | −3.57626275 |
| H1573 | WT | 6.357 | −3.301029996 |
| H2250 | WT | 0.001175 | −3.301029996 |
| H358 | WT | 0.002835 | −3.301029996 |
| H460 | WT | 2.914 | −3.301029996 |

### Example 10: Materials and Methods

### Cell Culture

The identity for all human cell lines in this study was confirmed by short tandem repeat (STR) analysis. In addition, all human cell lines were confirmed to be mycoplasma free using a PCR based assay (Genatlantis). HCT-116 cells (ATCC) were grown in DMEM medium with 10% FBS and 2 mM L-glutamine. Lenti-X 293T cells (Clontech) were cultured in DMEM medium with 10% FBS and 2 mM L-glutamine. H1155 cells (a gift from Adi Gazdar and John Minna, UT Southwestern) were cultured in RPMI media with 5% FBS and 2 mM L-glutamine. SF9 cells (a gift from Hongtao Yu, UT Southwestern) were cultured in SF900 II serum free media (Life Technologies).

### Chemicals

Indisulam was purchased from MedKoo Biosciences. Paclitaxel was purchased from Selleckchem. Bortezomib was purchased from Selleckchem. MLN-4924 was purchased from Active Biochem. Chloroquinoxaline sulfonamide (CQS) was obtained from NCI drug collection. Tasisulam was purchased from Adooq Bioscience. Acetozolamide and topiramate were purchased from Santa Cruz Biotechnology. The above compounds were all prepared as 10 mM stocks in DMSO and further diluted in DMSO to desirable concentrations. Auxin (3-indoleacetic acid/IAA) was purchased from Sigma and prepared as 100 mM stocks in DMSO.

### Forward Genetics of Drug Resistance in HCT-116 Cells

The methods for generating a population of barcoded HCT-116 cells were described before (20). To isolate indisulam resistant clones, ten 10 cm² plates of barcoded HCT-116 cells (1 million cells per plate) were treated with 1 μM indisulam for 2 weeks, with media change every 3-4 days. Clones that appeared after the selection was then expanded and barcode genotyped to identify six clones with unique barcodes. Whole-exome sequencing of six indisulam resistant clones was performed as described before (20). Source data for exome sequencing data is available under NCBI SRA accession SRP068238. Bortezomib resistant clones were isolated by selection with 5 nM bortezomib, and MLN4924 resistant clones with 40 nM MLN4924.

### Cell Viability Assay

12-point dose responses were performed on 96-well assay plates with cell plating (1500-4000 cell per well) on day 1, compound addition on day 2, and survival measurement on day 5. Compounds were diluted in DMSO before adding to the cells. Final DMSO concentration was 0.5%. Cell survival assay was performed using CellTiter-Glo Luminescent Cell Viability Assay kit (Promega) that measures cellular ATP content. Luminescence was recorded by EnVison multimode plate reader (Perkin Elmer). IC50 was determined with GraphPad Prism using baseline correction (by normalizing to DMSO control), the asymmetric (five parameter) equation and least squares fit.

Transient Transfection of RBM39 cDNA into HCT-116 Cells

RBM39 was PCR amplified from U2OS cDNA and cloned into pLVX IRES zsGreen1 (Clontech). Mutagenesis of RBM39 was performed using Q5 site-directed mutagenesis kit (NEB) and primers were designed using the NEBaseChanger web server (nebasechanger.neb.com/). For transient transfection, 0.1 million HCT-116 cells were seeded per well in a 12-well plate on day 1. On day 2, cells were transfected with 1 μg of plasmid (pLVX IRES zsGreen containing wild type or mutant RBM39). On day 3, 2 μM of indisulam was added to each well. On day 8, crystal violet staining was performed to visualize cells that survived indisulam treatment. For crystal violet staining, cells on plates were stained with 0.05% crystal violet, 1% formaldehyde in 1×PBS for 20 min at room temperature, followed by several rinses with deionized water to remove free stain.

### Knock-in of RBM39 G268V Allele

sgRNA targeting RBM39 (5′-aactgaagatatgcttcgt-3′) was cloned into the pSpCas9(BB)-2A-Puro (PX459) vector (Addgene 62988). For RBM39 G268V knock-in, 1 million HCT-116 or H1155 cells were nucleofected (using 4D-Nucleofector, Lonza) with PX459-RBM39 sgRNA and the single-stranded oligo 5′-AAAGCAAGACATACAGAAATTATGTAGTTATACTCAGATCAACAACCTACTGAAGA TTCATTGAAGAACCTGGACTTACTCTTCCAAAAGGCTCAAAGATCACACGAAGCATA TCTTCAGTTATGTTGAAGTGTAATGAGCCCACATAAAGCCTCATAGGTCCAGCACTT CCCTTTTGTAAATTGTTTGCCATTGCTGCA-3′ (SEQ ID NO: 13). Afterwards, cells were exposed to 2 μM indisulam for two weeks to select for cells with RBM39 G268V knock-in. Cells that survived indisulam treatment were recovered and confirmed to have the correct G268V genomic conversion via Sanger sequencing.

### Testing In Vivo Efficacy of Indisulam Using Mouse Xenograft Models

All animal work was approved by the Institutional Animal Care and Use Committee at UTSW. UTSW is accredited by the Association for Assessment and Accreditation of Laboratory Animal Care International (AAALAC). Animals were obtained from the UTSW Breeding Core Facility. Nod-Scid IL-2Rγ KO mice were implanted with 5 million parental HCT-116 cells or HCT-116 cells expressing RBM39 G268V in a volume of 0.1 ml in the left flank. Tumor volumes were measured every 2-4 days with calipers. Volume is calculated as (L*W²)*3.14)/6. When volumes reached ˜110 mm³, therapy was started with 25 mg/kg indisulam or vehicle control (3.5% DMSO, 6.5% Tween 80, 90% saline; 0.2 ml/mouse) once a day for eight days. Average maximal weight loss from start of therapy to 1 day post final dose was 5.2% for parental indisulam, 2.5% for parental vehicle, 8.4% for G268V indisulam, and 5.9% for G268V vehicle.

### Cell Lysis and RBM39 Degradation In Vivo

Cells exposed to DMSO (0.1%) or various concentrations of indisulam were washed once with PBS, and solubilized in 1% SDS, with benzonase (Sigma E1014) diluted 1:10,000 in buffer A (50 mM HEPES 7.4, 10 mM KCl and 2 mM MgCl₂). Proteins were resolved on SDS-PAGE and transferred to 0.5 μm nitrocellulose membranes. Membranes were blocked in 5% milk TBST for 30 min, and then anti-RBM39 antibodies (HPA001591, Sigma-Aldrich) were added in 5% milk (1:5000) and incubated on a shaker overnight at 4° C. After three 5 min washes with TBST, membranes were incubated with HRP-conjugated goat anti-rabbit secondary antibody (Bio-Rad) for 2 hr at room temperature. After three 5 min washes with TBST, membranes were developed with ECL (enhanced chemiluminescence) substrates.

### C-Terminal Tagging of Endogenous RBM39

Five independent guide RNAs targeting the genomic region surrounding the RBM39 stop codon were cloned into pLX sgRNA (Addgene 50662). Repair templates were constructed in a pGEM-T Easy vector and contained two 2 kilobase homology arms matching upstream and downstream sequences of the genomic locus, sequences encoding different tags, as well as an IRES Neo cassette flanked by two LoxP sites. For endogenous tagging, 1 million HCT-116 were nucleofected (using 4D-Nucleofector, Lonza) with a mixture of the five guide RNA plasmids (200 ng each), 1 μg of PX459 (containing Cas9), and 1 μg of repair template. Selection with 1 mg/ml G418 was performed until clones appear. Multiple clones were isolated and successful integration of C-terminal tag was validated by western blotting with anti-RBM39 and HRP conjugated anti-FLAG (Sigma-Aldrich, A8592, 1:5000). Knock-in tagging in H1155 was similar except that 0.5 million cells were transfected using Fugene HD (Promega).

### Auxin Induced Degradation of RBM39

TIR1 cDNA with a V5 tag sequence appended to its 3′ end was cloned into pLVX IRES zsGreenland packaged into lentivirus to infect both parental HCT-116 cells and RBM39-AID-3xFLAG knock-in cells. Following lentiviral delivery of TIR1, cells with top 5% strongest zsGreen expression were sorted by FACS. TIR1-V5 expression was confirmed by western blot with HRP conjugated anti-VS (Sigma-Aldrich, V2260, 1:5000). For examination of RBM39-AID-3xFLAG degradation, cells were treated with 500 μM IAA for 4 hours before being lysed for western blotting. For IAA dose response curves, cells were treated with various concentrations of IAA for 72 hours and ATP levels were quantified using Cell-Titer Glo (Promega).

### Immunoprecipitation of RBM39 Complex

Anti-Flag M2 antibody (Sigma-Aldrich) was coupled to Dynabeads M-270 epoxy beads (Life Technologies) at the ratio of 10 μg of antibody/mg beads. H1155 RBM39-3xFLAG cells grown on twenty 15 cm² plates were treated with DMSO or 10 μM indisulam for 2 hours, detached from plates by scraping, washed in PBS, and then frozen in liquid nitrogen. Frozen cells were then pulverized in the a cryomill (Retsch) with one 25 mm steel ball and five cycles of three min at 25 Hz with intermittent cooling in liquid nitrogen. 800 mg of grinded cell powder for each sample was resuspended with 4 ml of IP buffer (50 mM NaCl, 50 mM NaPO₄, 50 mM NaCitrate, 20 mM HEPES pH 7.4, 0.1% Tween, and 1× SIGMAFAST Protease Inhibitor (Sigma)) supplemented with DMSO or 10 μM indisulam. The lysates were centrifuged at 8000 g for 5 min at 4° C. 12 mg of conjugated Anti-Flag magnetic beads were mixed with clarified lysates for 5 min at 4° C. on a rotating platform, followed by three washes with IP buffer. Bound proteins were eluted with 1 mg/ml 3xFLAG peptide, followed by SDS-PAGE on a 10% Bis Tris gel. After brief electrophoresis, bands containing all the proteins were cut with clean blades. For western blotting of co-immunoprecipitated proteins, anti-DDA1 (ProteinTech, 14995-1-AP, 1:1000), anti-DDB1 (Abcam, ab109027, 1;10000), anti-CUL4A (Cell Signaling, 2699, 1:1000), anti-CUL4B (ProteinTech, 12916-1-AP, 1:1000) were used.

### Protein Identification by LC-MS/MS

Excised gel bands were cut into approximately 1 mm³ pieces. Gel pieces were then subjected to a modified in-gel trypsin digestion procedure (46). Gel pieces were washed and dehydrated with acetonitrile for 10 min. followed by removal of acetonitrile. Pieces were then completely dried in a speed-vac. Rehydration of the gel pieces was with 50 mM ammonium bicarbonate solution containing 12.5 ng/μl modified sequencing-grade trypsin (Promega, Madison, Wis.) at 4° C. After 45 min., the excess trypsin solution was removed and replaced with 50 mM ammonium bicarbonate solution to just cover the gel pieces. Samples were then placed in a 37° C. room overnight. Peptides were later extracted by removing the ammonium bicarbonate solution, followed by one wash with a solution containing 50% acetonitrile and 1% formic acid. The extracts were then dried in a speed-vac (˜1 hr). The samples were then stored at 4° C. until analysis. On the day of analysis the samples were reconstituted in 5-10 μl of HPLC solvent A (2.5% acetonitrile, 0.1% formic acid). A nano-scale reverse-phase HPLC capillary column was created by packing 2.6 μm C18 spherical silica beads into a fused silica capillary (100 μm inner diameter x ˜30 cm length) with a flame-drawn tip (47). After equilibrating the column each sample was loaded via a Famos auto sampler (LC Packings, San Francisco Calif.) onto the column. A gradient was formed and peptides were eluted with increasing concentrations of solvent B (97.5% acetonitrile, 0.1% formic acid). As peptides eluted they were subjected to electrospray ionization and then entered into an LTQ Orbitrap Velos Pro ion-trap mass spectrometer (Thermo Fisher Scientific, Waltham, Mass.). Peptides were detected, isolated, and fragmented to produce a tandem mass spectrum of specific fragment ions for each peptide. Peptide sequences (and hence protein identity) were determined by matching protein databases with the acquired fragmentation pattern by the software program, Sequest (Thermo Fisher Scientific, Waltham, Mass.) (48). All databases include a reversed version of all the sequences and the data was filtered to between a one and two percent peptide false discovery rate.

### Expression of Dominant Negative Cullins

Cullin ORF clones were obtained from Ultimate ORF Lite human cDNA collection (Life Technologies) and used as PCR template to amplify N-terminal fragments of CUL1 (a.a.1-452), 2 (a.a.1-422), 3 (a.a.1-418), 4A (a.a.1-440), 4B (a.a.1-594), 5 (a.a.1-441). Sequence encoding a V5 tag was appended to the 3′ end of each amplified CULLIN fragments. These fragments were cloned into pLVX IRES zsGreen1 to create dominant negative Cullin constructs. For transient expression, 0.5 million 293T cells were transfected with 0.5 μg of each construct (2.5 μg for CUL2). After 24 hr, transfected cells were treated with DMSO or 2 μM indisulam for 8 hr. Cells were lysed in 1% SDS Buffer A with benzonase and subjected to western blot.

### CRISPR Mediated Inactivation of DDB1, DDA1, and DCAF15

Two independent guide RNAs targeting DDB1, DDA1, DCAF15 were cloned into PX459. 0.2 million H1155 RBM39-AcGFP cells were transfected with PX459 sgRNA constructs and selected with 2 μg/ml puromycin to enrich for transfected cells. Four days post transfection, cells were split into two wells, with one well treated with DMSO, and the other well treated with 2 μM indisulam. Cells were then trypsinized and the expression of RBM39-AGFP was quantified with flow cytometry. H1155 cells transfected with DCAF15 guide RNA were sparsely plated on 10 cm² plates and clones were isolated. Genomic DNA sequence flanking the cut site were amplified and sequenced to identify DCAF15−/− clones.

Ectopic expression of 3XFLAG tagged CUL4A, CUL4B, DCAF15, and DDB1

Full-length cDNAs encoding CUL4A, CUL4B, DCAF15, and DDB1 were cloned into pLVX IRES Puro. Sequence encoding a 3XFLAG tag was appended to the 3′ end of each cDNA by PCR. Lentiviral packaging of the resulting plasmids was performed by co-transfecting the plasmids with psPAX2 (Addgene 12260) and pMD2.G (Addgene 12259) into lenti-X 293T cells. Media collected from transfected lenti-X 293T cells was used to infect HCT-116 cells. Cells stably expressing 3XFLAG tagged CUL4A, CUL4B, DCAF15, or DDB1 after three days of selection with 2 μg/ml puromycin.

### In Vivo Polyubiquitination of RBM39

cDNAs encoding RBM39-3xFLAG, RBM39 G268V-3xFLAG, and DCAF15-V5 were cloned into the pCDNA3.1+vector. pCMV 8XHis Ub was a gift from William Kaelin (Dana Farber Cancer Institute). 0.4 million 293T cells were seeded per well on 6-well plates and allowed to attach overnight. 250 ng of pCMV 8XHis Ub, 500 ng of pCDNA3-DCAF15-V5, 10 ng of either pCDNA3-RBM39-3xFLAG or pCDNA3-RBM39 G268V-3xFLAG were transfected. 40 hours later, cells were pretreated with 100 nM bortezomib for 2 hours, and then treated with DMSO or 2 μM indisulam for 4 hours. Cells were lifted from the plates by pipetting, pelleted by centrifugation, and washed once with PBS. Purification of His-Ub tagged proteins was performed as described (5).

### Expression and Purification of RBM39A150 and DCAF15-GST/DDB1

RBM39 lacking the first 150 amino acids after the start codon (RBM39Δ150) was cloned into the pFastBac1 (Life Technologies) vector. Site-directed Mutagenesis of RBM39 was performed using Q5 site-directed mutagenesis kit (NEB). DCAF15-GST (C-terminal GST tag) and DDB1 were also cloned into pFastBac1. The production of baculovirus was carried out following vendor's instructions. Protein was expressed in SF9 cells by infection with each protein specific baculovirus, and cells were harvested 48 hr later and frozen in liquid nitrogen. For purification of RBM39Δ150, frozen cells were pulverized by cryomill and resuspended in lysis buffer (40 mM Tris, pH 7.5, 225 mM NaCl, 1× SIGMAFAST Protease inhibitor). The lysate was passed through a 22 gauge needle 2 times followed by ultracentrifugation at 25000 rpm for 30 min. Clarified lysate was incubated with Ni-NTA agarose (Qiagen) for 30 min with rotation at 4° C. Resin and bound protein were washed with wash buffer 1 (40 mM Tris, pH 7.5, 225 mM NaCl) followed by wash buffer 2 (40 mM Tris, pH 7.5, 20 mM imidazole, 225 mM NaCl). Proteins were eluted with elution buffer (40 mM Tris, pH 7.5, 300 mM imidazole, 225 mM NaCl) and concentrated to 0.5 ml. Concentrated proteins were then fractionated on Superdex 200 Increase 10/300 GL (GE Healthcare). RBM39Δ150 and mutants eluted at 14.8 ml after injection and were stored at −80° C. in 40 mM Tris, pH 7.5, 225 mM NaCl. For purification of DCAF15-GST/DDB1, Sf9 cells co-infected with DCAF15-GST and DDB1 baculoviruses were lysed in PBS supplemented with 0.1% NP40, 1× SIGMAFAST Protease inhibitors, and 0.5 mM PMSF. The lysate was passed through a 22 gauge needle 2 times followed by centrifugation at 7000 rpm for 15 min. Clarified lysate was incubated with Glutathione Sepharose 4B resin (GE healthcare) for 30 min with rotation at 4° C. Resin was washed with PBS for three times before elution with 50 mM Tris, 10 mM glutathione, pH 8.

### RBM39Δ150 Pulldown Assay

HCT-116 cells stably expressing DDB1-3xFLAG, CUL4A-3xFLAG, CUL4B-3xFLAG, or to DCAF15-3xFLAG were lysed in 1×PBS, 0.1% NP40, and 1× SIGMAFAST protease inhibitor (1 ml per 15 cm² plate). After clarification by centrifugation at 16000 g for 10 min, lysates were mixed with anti-FLAG magnetic beads (2 mg beads per 0.5 ml of lysate) and rotated at 4° C. for 10 min. Beads were washed three times with 5×PBS, 0.1% NP40 and once with 1×PBS, 0.1% NP40. Subsequently, beads were mixed with 100 nM RBM39Δ150 and 1 μM indisulam (or DMSO) and incubated with agitation at 4° C. for 30 min. After three washes with 1×PBS, 0.1% NP40, proteins were eluted with 1 mg/ml 3xFLAG peptide and subjected to SDS-PAGE and western blotting. For pulldown assays with mutant RBM39 proteins and different concentrations of sulfonamide compounds, DCAF15-3xFLAG was isolated from 293T cells stably expressing DCAF15-3xFLAG. 100 nM of RBM39Δ150 proteins and various concentrations of indisulam, CQS, or tasisulam were used.

### Indisulam Dependent Co-Fractionation of DCAF15-DDB1 and RBM39

1.76 μM RBM39Δ150 was mixed with 0.624 μM DCAF15-GST/DDB1 in the presence or absence of 10 μM indisulam. The mixture was incubated for 1 hour with rotation at 4° C. Samples were fractioned by Superose 6 increase 10/300 GL using 50 mM Tris (pH 7.5), 100 mM NaCl supplemented with DMSO (1:1000) or indisulam (10 μM). 1 ml fractions were collected and analyzed by SDS-PAGE and coomassie blue staining.

### Mass Spec Quantification of Indisulam in RBM39 DCAF15 Complex

RBM39-3xFLAG was immunopurified from HCT-116 cells expressing RBM39-3xFLAG (endogenous tag) using the cryomill procedure. DCAF15-3xFLAG was purified from 293T cells stably expressing DCAF15-3xFLAG through lentiviral transduction. After three stringent washes with 5×PBS, 0.1% NP40, beads with bound proteins were mixed with 1 μM RBM39 Δ150 and 10 μM indisulam and incubated with agitation at 4° C. for 30 min. Afterwards, beads were washed twice with 1×PBS, 0.1% NP40 and twice with 1×PBS. Proteins were eluted with 50 μl of 1×PBS containing 1 mg/ml 3xFLAG peptide, and bound indisulam was extracted by addition of a 50 μl of methanol containing 0.2% formic acid and an internal standard (200 ng/ml tolbutamide, Sigma). Supernatant containing the eluted indisulam was evaluated by LC-MS/MS using a Sciex 4000 QTrap mass spectrometer coupled to a Shimadzu Prominence LC (20 μl injection). The mass spectrometer was run in MRM (multiple reaction monitoring mode) to detect indisulam by following the parent to fragment transition 383.9 to 319.9 (negative mode; M-H⁻). The tolbutamide internal standard (IS) was detected using the transition 269.1 to 169.9 (negative mode; M-H⁻). An Agilent C18 XDB 5-μm packing column (50×4.6 mm) was used for chromatography with the following conditions—buffer A: H₂O+0.1% formic acid; buffer B: methanol+0.1% formic acid, 0-1.5 min 3% (vol/vol) B, 1.5-2.0 min gradient to 100% (vol/vol) B, 2.0-3.2 min 100% (vol/vol) B, 3.2-3.5 min gradient to 3% (vol/vol) B, and 3.5-4.5 min 3% (vol/vol) B. Compound levels were quantitated using a standard curve prepared by spiking a 1:1 mixture of elution buffer and methanol containing a final concentration of 0.1% formic acid and 100 ng/ml tolbutamide IS with various concentrations of indisulam. The limit of quantitation (lowest quantifiable point on the standard curve with accuracy within 20% upon back calculation and at least 3-fold above background) for this method was 50 pg/ml.

### Measurement of RBM39-Nluc Degradation by Nano-Glo Luciferase Assay

NanoLuc was knocked-in to the 3-prime end of the RBM39 gene using the procedure described above yielding H1155 RBM39-Nluc cells. 4000 cells were plated in triplicate for a 12-point dose response of either indisulam, tasisulam, or CQS in 96-well assay plates on day 1 followed by compound addition on day 2, and Nano-Glo luciferase assay (Promega) on day 3. Luminescence was recorded by EnVision multimode plate reader (Perkin Elmer). IC50 was determined with GraphPad Prism using baseline correction (by normalizing to DMSO control), the asymmetric (five parameter) equation and least squares fit.

### RNA-Seq and Data Processing

Parental HCT-116 cells and cells harboring RBM39 G268V mutation were treated with 2 μM indisulam for 0, 6, and 12 hours. RNAs were extracted using Quick-RNA MiniPrep kit (Zymo Research). After cleaning up genomic DNA contaminant with Turbo DNase (Life Technologies) and rRNA depletion, strand-specific cDNA libraries were generated using the TruSeq Stranded mRNA Sample Prep Kit (IIlumina) and were subjected to high throughput sequencing using

HiSeq2500 platform (Illumina) with 100-bp single-end reads. NGS QC Toolkit (v2.3.1) (49) was used to check the sequencing quality, and high-quality reads were aligned by TopHat (v2.0.8) (50) to human reference genome (hg19) along with the gene annotation data downloaded from Illumina's iGenomes (support.illumina.com/sequencing/sequencing_software/igenome.ilmn). The insert-size metrics required in TopHat execution and the correctness of strand specificity were estimated by mapping reads to human transcript sequences using Bowtie (v2.1.0) (51) and Picard-tools (v1.99) (picard.sourceforge.net). The htseq-count script distributed with the HTSeq Python package (0.6.1) (pypi.python.org/pypi/HTSeq) was employed for counting reads in genes considering the coding strands. Source data for RNA sequencing data is available under NCBI SRA accession SRP096666.

### Bioinformatic Analysis of Splicing Changes

For the stringent detection of differential skipped exons and retained introns, a custom program called SpliceFisher was developed and it is downloadable at github.com/jiwoongbio/SpliceFisher. Exonic and intronic regions were defined as annotated in the reference genome. To estimate differential exon skipping events, the numbers of exon-junction reads (panels a and b in FIG. 16) and exon-skipping reads (panel c in FIG. 16) observed in a sample were compared to the numbers observed in another sample. For differential intron retention, the numbers of exon-intron reads (panel a and b in FIG. 16) and exon-exon reads (panel c in FIG. 16) were used instead. Additionally, the number of reads mapped to the target region (panel d in FIG. 16) and other exonic regions of the gene (panel e in FIG. 16) were also compared between samples. The read counts were evaluated by multiple Fisher's exact tests in three two-by-two tables using R and then the p-values were adjusted using FDR by each comparison type. Differential alternative splicing events were identified with all three adjusted p-values less than 0.01. The differential alternative splice sites and mutually exclusive exons were additionally detected by a similar method using the numbers of splicing reads supporting each junction.

RBM39 siRNA Knockdown

For siRNA transfection, 200,000 cells plated in a well of a 6-well plate were transfected with 100 pmol of siRNAs using 5 μL of Lipofectamine RNAiMax (Life Technologies). 40 hours after transfection, cells were collected for RNA extraction using Quick-RNA Miniprep kit (Zymo Research). The control siRNA targeting firefly luciferase was purchased from Dharmacon. Two siRNAs targeting RBM39 were purchased from Sigma. The following siRNA sequences were used. RBM39 siRNA 1: 5′-UCGAUCUCGACUUCUUGAG-3′ (SEQ ID NO: 1); RBM39 siRNA 2: 5′-UAUGUUGAAGUGUAAUGAG-3′ (SEQ ID NO: 2).

### PCR Validation of Splicing Changes

RNAs isolated from indisulam or siRNA treated cells were first treated with Turbo DNase (Life Technologies) to remove contaminating genomic DNA. After RNA cleanup with Quick-RNA MiniPrep kit (Zymo Research), 500 ng of total RNAs was converted into cDNAs with Multiscribe reverse transcriptase (Life Technologies). Exon skipping in TRIM27 was visualized by PCR amplification with 5′-CCTGAACCTTGGATCACACC-3′ (SEQ ID NO: 3) and 5′-GCAGGTCCTGTTGGAGGTAA-3′ (SEQ ID NO: 4) from cDNAs. For intron retention in RBM3, cDNAs were analyzed by a Bio-Rad thermocycler using Power Sybr Green PCR master mix (Applied Biosystems). Relative RNA levels were calculated based on 2^{-ct} method using Cyclophilin mRNA for normalization. The following primers were used for quantification of mRNA levels:

Cyclophilin:
(SEQ ID NO: 5)
5′-TGCCATCGCCAAGGAGTAG-3′
and
(SEQ ID NO: 6)
5′-TGCACAGACGGTCACTCAAA-3′
RBM3 exon6-intron6:
(SEQ ID NO: 7)
5′-GACCGCTACTCAGGAGGAAA-3′
and
(SEQ ID NO: 8)
5′-AGGTATGAGGGCAGCAGAAA-3′;
RBM3 exon6-exon7:
(SEQ ID NO: 9)
5′-GGTGGTTATGACCGCTACTCA-3′
and
(SEQ ID NO: 10)
5′-AGCCATTTGGAAGGACGA-3′.

For measurement of RBM39 mRNA levels, the following primers were used:

RBM39:
(SEQ ID NO: 11)
5′-GTCGATGTTAGCTCAGTGCCTC-3′
and
(SEQ ID NO: 12)
5′-ACGAAGCATATCTTCAGTTATG-3′

Correlating Indisulam Sensitivity with Basal Gene Expression and Copy Number Variation

Datasets of indisulam AUC (area under the curve), basal gene expression, and copy number variation were described in (41). Pearson correlation coefficients were calculated between gene either expression or copy number variation and AUCs across all HL lineage cancer cell lines (tables 3 and 4).

### Discussion

Indisulam, tasisulam, and CQS, are all aryl sulfonamides (hereafter collectively referred to as SPlicing inhibitor suLfonAMides or SPLAMs) that mediate an interaction between the DDB1/CUL4 receptor DCAF15 and RBM39, as a neo-substrate. The mechanism of action of SPLAMs is analogous to that of IMiDs, anti-cancer drugs that recruit neo-substrates including Ikaros, Aiolos, and CK1α to CRBN, a different DDB1/CUL4 associated receptor (4-6). X-ray crystallography of an IMiD called lenalidomide in complex with CUL4-CRBN and CK1α reveal that lenalidomide acts as a bridge by making contacts with both CRBN and CK1α (42). This is the same mechanism by which the plant hormone auxin acts like “molecular glue” to enhance an interaction between the E3 ubiquitin ligase receptor TIR1 and its substrates (43). We only detected indisulam binding to complexes composed of both RBM39 and DCAF15, but neither protein alone, suggesting that SPLAMs might also act like molecular glue (FIG. 4, panel E). We identified multiple mutations on one face of an α-helix in the RRM2 domain of RBM39 that reduce the ability of SPLAMs to form the ternary complex (FIG. 1, panels D and E). We suspect that in the ternary complex, this helix may form a direct point of interaction between DCAF15 and RBM39 or between RBM39 and SPLAMs. X-ray crystallography of the RBM39-SPLAM-DCAF15 complex will be necessary to determine the precise role of these amino acid residues and whether the SPLAMs do indeed act as a glue to enhance interaction between RBM39 and DCAF15.

Conceivably the activity of SPLAMs could be expanded to include other neo-substrates as is the case for IMiDs. We did not detect any change in the basal levels of RBM39 in cells that either overexpress or lack DCAF15, suggesting that RBM39 is not an endogenous substrate of DCAF15. The neo-morphic activity of IMiDs can be tuned to influence specific substrates depending on their chemical composition. For example, all three IMiDs can recruit IKZF1 and IKZF3 to CUL4-CRBN; however, only lenalidomide can recruit CK1α to CUL4-CRBN (6). The basis for this specificity is that lenalidomide contains an amino group that makes a key contact specific for CK1α. In another example of how chemical structure can influence neo-substrate preference, CC-885 was identified in a screen of IMiD derivatives to catalyze the recruitment of GSTP1 to CUL4-CRBN, a translation termination factor essential for myeloid leukemia cell proliferation (44). An important area of investigation for the future will be determining whether SPLAM derivatives can recruit neo-substrates other than RBM39 to CUL4-DCAF15.

The proposed mechanism of action of SPLAMs potentially has implications for the future use of these molecules in cancer treatment. Indisulam, tasisulam, and CQS have all been tested in either phase II or phase III clinical trials of patients with metastatic cancer. These compounds did not advance because of their limited efficacy rather than unacceptable toxicity. In all of these trials, fewer than 10% patients had a clinical response and the objective response rates (which include stable disease) were less than 30%. We now know that the anti-cancer activity of SPLAMs is proportional to DCAF15 expression and RBM39 dependence. Improved efficacy might be seen if future clinical trials are restricted to cancers that express high levels of DCAF15. Indisulam sensitivity correlates with cancer cells that are derived from the hematopoietic and lymphoid lineages suggesting that they may be especially dependent on RBM39. We noted a significant correlation between both DCAF15 expression and gene copy number with indisulam sensitivity among these cancer cell lines. Taken together, our findings suggest that future clinical trials with SPLAMs should focus on leukemias and lymphomas showing high expression levels of DCAF15.

In summary, SPLAMs provide a strategy to target RNA splicing in cancer. Cancer genome sequencing efforts have identified mutations in canonical splicing factors that include U2AF1, SF3B1, SRSF2, and ZRSR2 (2). These mutations are most often identified in myelodysplastic syndrome, chronic lymphocytic leukemia, and acute myeloid leukemia, again highlighting the importance of RNA splicing in hematopoietic and lymphoid malignancies. That these cancers are also more likely to be dependent on RBM39 suggests that RBM39 may be a critical factor in these RNA splicing programs. Therapeutic strategies that target splicing in hematopoietic and lymphoid malignancies have thus far centered on small molecule inhibitors of proteins important for splicing most introns. One example is spliceostatin, a potent inhibitor of the canonical splicing factor SF3B1 (45). Spliceostatin has shown some efficacy in clinical trials but has also been associated with adverse events (1). Drugs inducing degradation of RBM39, in contrast, appear to influence the splicing of only a subset of pre-mRNAs. As such, the SPLAM compounds may offer the opportunity to selectively target splicing pathways important for cancer cell growth.

### REFERENCES

1. Lee S C & Abdel-Wahab O (2016) Therapeutic targeting of splicing in cancer. Nat Med 22(9):976-986.
2. Dvinge H, Kim E, Abdel-Wahab O, & Bradley R K (2016) RNA splicing factors as oncoproteins and tumour suppressors. Nat Rev Cancer 16(7):413-430.
3. Ito T, et al. (2010) Identification of a primary target of thalidomide teratogenicity. Science 327(5971):1345-1350.
4. Kronke J, et al. (2014) Lenalidomide causes selective degradation of IKZF1 and IKZF3 in multiple myeloma cells. Science 343(6168):301-305.
5. Lu G, et al. (2014) The myeloma drug lenalidomide promotes the cereblon-dependent destruction of Ikaros proteins. Science 343(6168):305-309.
6. Kronke J, et al. (2015) Lenalidomide induces ubiquitination and degradation of CK1alpha in del(5q) MDS. Nature 523(7559):183-188.
Owa T, et al. (1999) Discovery of novel antitumor sulfonamides targeting G1 phase of the cell cycle. J Med Chem 42(19):3789-3799.
8. Fukuoka K, et al. (2001) Mechanisms of action of the novel sulfonamide anticancer agent E7070 on cell cycle progression in human non-small cell lung cancer cells. Invest New Drugs 19(3):219-227.
9. Ozawa Y, et al. (2001) E7070, a novel sulphonamide agent with potent antitumour activity in vitro and in vivo. Eur J Cancer 37(17):2275-2282.
10. Punt C J, et al. (2001) Phase I and pharmacokinetic study of E7070, a novel sulfonamide, given at a daily times five schedule in patients with solid tumors. A study by the EORTC-early clinical studies group (ECSG). Ann Oncol 12(9):1289-1293.
11. Raymond E, et al. (2002) Phase I and pharmacokinetic study of E7070, a novel chloroindolyl sulfonamide cell-cycle inhibitor, administered as a one-hour infusion every three weeks in patients with advanced cancer. J Clin Oncol 20(16):3508-3521.
12. Dittrich C, et al. (2003) Phase I and pharmacokinetic study of E7070, a chloroindolyl-sulfonamide anticancer agent, administered on a weekly schedule to patients with solid tumors. Clin Cancer Res 9(14):5195-5204.
13. Terret C, et al. (2003) Phase I clinical and pharmacokinetic study of E7070, a novel sulfonamide given as a 5-day continuous infusion repeated every 3 weeks in patients with solid tumours. A study by the EORTC Early Clinical Study Group (ECSG). Eur J Cancer 39(8): 1097-1104.
14. Haddad R I, et al. (2004) A phase II clinical and pharmacodynamic study of E7070 in patients with metastatic, recurrent, or refractory squamous cell carcinoma of the head and neck: modulation of retinoblastoma protein phosphorylation by a novel chloroindolyl sulfonamide cell cycle inhibitor. Clin Cancer Res 10(14):4680-4687.
15. Smyth J F, et al. (2005) Phase II study of E7070 in patients with metastatic melanoma. Ann Oncol 16(1):158-161.
16. Yamada Y, et al. (2005) Phase I pharmacokinetic and pharmacogenomic study of E7070 administered once every 21 days. Cancer Sci 96(10):721-728.
17. Talbot D C, et al. (2007) A randomized phase II pharmacokinetic and pharmacodynamic study of indisulam as second-line therapy in patients with advanced non-small cell lung cancer. Clin Cancer Res 13 (6):1816-1822.
18. Abbate F, Casini A, Owa T, Scozzafava A, & Supuran C T (2004) Carbonic anhydrase inhibitors: E7070, a sulfonamide anticancer agent, potently inhibits cytosolic isozymes I and II, and transmembrane, tumor-associated isozyme IX. Bioorg Med Chem Lett 14(1):217-223.
19. Wacker S A, Houghtaling B R, Elemento O, & Kapoor T M (2012) Using transcriptome sequencing to identify mechanisms of drug action and resistance. Nat Chem Biol 8(3):235-237.
20. Han T, et al. (2016) The antitumor toxin CD437 is a direct inhibitor of DNA polymerase alpha. Nat Chem Biol 12(7):511-515.
21. Gray W M, Kepinski S, Rouse D, Leyser O, & Estelle M (2001) Auxin regulates SCF(TIR1)-dependent degradation of AUX/IAA proteins. Nature 414(6861):271-276.
22. Natsume T, Kiyomitsu T, Saga Y, & Kanemaki M T (2016) Rapid Protein Depletion in Human Cells by Auxin-Inducible Degron Tagging with Short Homology Donors. Cell Rep 15(1):210-218.
23. Petroski M D & Deshaies R J (2005) Function and regulation of cullin-RING ubiquitin ligases. Nature reviews. Molecular cell biology 6(1):9-20.
24. Ohh M, et al. (2002) An intact NEDD8 pathway is required for Cullin-dependent ubiquitylation in mammalian cells. EMBO Rep 3(2):177-182.
25. Soucy T A, et al. (2009) An inhibitor of NEDD8-activating enzyme as a new approach to treat cancer. Nature 458(7239):732-736.
26. Toth J I, Yang L, Dahl R, & Petroski M D (2012) A gatekeeper residue for NEDD8-activating enzyme inhibition by MLN4924. Cell Rep 1(4):309-316.
27. Jin J, Arias E E, Chen J, Harper J W, & Walter J C (2006) A family of diverse Cul4-Ddb1-interacting proteins includes Cdt2, which is required for S phase destruction of the replication factor Cdt1. Molecular cell 23(5):709-721.
28. Ye Y & Rape M (2009) Building ubiquitin chains: E2 enzymes at work. Nature reviews. Molecular cell biology 10(11):755-764.
29. Emanuele M J, et al. (2011) Global identification of modular cullin-RING ligase substrates. Cell 147(2):459-474.
30. Jin J, Ang X L, Shirogane T, & Wade Harper J (2005) Identification of substrates for F-box proteins. Methods Enzymol 399:287-309.
31. Angers S, et al. (2006) Molecular architecture and assembly of the DDB1-CUL4A ubiquitin ligase machinery. Nature 443(7111):590-593.
32. Rigas J R, et al. (1992) Phase I clinical and pharmacological study of chloroquinoxaline sulfonamide. Cancer Res 52(23):6619-6623.
33. Miller V A, et al. (1997) Phase II trial of chloroquinoxaline sulfonamide (CQS) in patients with stage III and IV non-small-cell lung cancer. Cancer Chemother Pharmacol 40(5):415-418.
34. Bekaii-Saab T S, et al. (2006) A phase II study of chloroquinoxaline sulfonamide (CQS) in patients with metastatic colorectal carcinoma (MCRC). Invest New Drugs 24(4):343-346.
35. Haritunians T, Gueller S, O'Kelly J, Ilaria R, Jr., & Koeffler H P (2008) Novel acyl sulfonamide LY573636-sodium: effect on hematopoietic malignant cells. Oncol Rep 20(5):1237-1242.
36. Kirkwood J M, et al. (2011) A phase 2 study of tasisulam sodium (LY573636 sodium) as second-line treatment for patients with unresectable or metastatic melanoma. Cancer 117(20):4732-4739.
37. Simon G R, et al. (2011) A phase I study of tasisulam sodium (LY573636 sodium), a novel anticancer compound in patients with refractory solid tumors. Cancer Chemother Pharmacol 68(5):1233-1241.
38. Imai H, Chan E K, Kiyosawa K, Fu X D, & Tan E M (1993) Novel nuclear autoantigen with splicing factor motifs identified with antibody from hepatocellular carcinoma. J Clin Invest 92(5):2419-2426.
39. Basu A, et al. (2013) An interactive resource to identify cancer genetic and lineage dependencies targeted by small molecules. Cell 154(5):1151-1161.
40. Seashore-Ludlow B, et al. (2015) Harnessing Connectivity in a Large-Scale Small-Molecule Sensitivity Dataset. Cancer Discov 5(11):1210-1223.
41. Rees M G, et al. (2016) Correlating chemical sensitivity and basal gene expression reveals mechanism of action. Nat Chem Biol 12(2):109-116.
42. Petzold G, Fischer E S, & Thoma N H (2016) Structural basis of lenalidomide-induced CK1alpha degradation by the CRL4(CRBN) ubiquitin ligase. Nature 532(7597):127-130.
43. Tan X, Calderon-Villalobos, L. I., Sharon, M., Zheng, C., Robinson, C. V., Estelle, M., Zheng, N. (2007) Mechanism of auxin perception by the TIR1 ubiquitin ligase. Nature 446(7136):640-645.
44. Matyskiela M E, et al. (2016) A novel cereblon modulator recruits GSPT1 to the CRL4(CRBN) ubiquitin ligase. Nature 535(7611):252-257.
45. Kaida D, et al. (2007) Spliceostatin A targets SF3b and inhibits both splicing and nuclear retention of pre-mRNA. Nat Chem Biol 3(9):576-583.
46. Shevchenko A, Wilm, M., Vorm, O., Mann, M. (1996) Mass spectrometric sequencing of proteins silver-stained polyacrylamide gels. Anal Chem 68(5):850-858.
47. Peng J, Gygi, S. P. (2001) Proteomics: the move to mixtures. J Mass Spectrom 36(10):1083-1091.
48. Eng J K, McCormack, A. L., Yates, J. R. (1994) An approach to correlate tandem mass spectral data of peptides with amino acid sequences in a protein database. J Am Soc Mass Spectrom 5(11):976-989.
49. Patel R K & Jain M (2012) NGS QC Toolkit: a toolkit for quality control of next generation sequencing data. PloS one 7(2):e30619.
50. Kim D, et al. (2013) TopHat2: accurate alignment of transcriptomes in the presence of insertions, deletions and gene fusions. Genome biology 14(4):R36.
51. Langmead B & Salzberg S L (2012) Fast gapped-read alignment with Bowtie 2. Nature methods 9(4):357-359.

TABLE 1

| Reference | Gene Symbol | MWT(kDa) | indisulam unique | DMSO unique | indisulam total | DMSO total |
|---|---|---|---|---|---|---|
| Q13620_CUL4B_HUMAN | CUL4B | 103.92 | 15 | 0 | 16 | 0 |
| Q7Z353_HDX_HUMAN | HDX | 77.16 | 14 | 0 | 15 | 0 |
| Q66K64_DCA15_HUMAN | DCAF15 | 66.42 | 10 | 0 | 11 | 0 |
| P35637_FUS_HUMAN | FUS | 53.39 | 6 | 0 | 6 | 0 |
| P62195_PRS8_HUMAN | PSMC5 | 45.6 | 6 | 0 | 6 | 0 |
| Q7L014_DDX46_HUMAN | DDX46 | 117.29 | 6 | 0 | 6 | 0 |
| O00231_PSD11_HUMAN | PSMD11 | 47.43 | 5 | 0 | 5 | 0 |
| O75688_PPM1B_HUMAN | PPM1B | 52.61 | 5 | 0 | 5 | 0 |
| P31946_1433B_HUMAN | YWHAB | 28.06 | 4 | 0 | 5 | 0 |
| P06753_TPM3_HUMAN | TPM3 | 32.93 | 4 | 0 | 4 | 0 |
| P30101_PDIA3_HUMAN | PDIA3 | 56.75 | 4 | 0 | 4 | 0 |
| P60174_TPIS_HUMAN | TPI1 | 30.77 | 4 | 0 | 4 | 0 |
| P62937_PPIA_HUMAN | PPIA | 18 | 4 | 0 | 4 | 0 |
| Q5HYB6_Q5HYB6_HUMAN | DKFZp686J1372 | 27.16 | 4 | 0 | 4 | 0 |
| Q9BW61_DDA1_HUMAN | DDA1 | 11.83 | 4 | 0 | 4 | 0 |
| P01857_IGHG1_HUMAN | IGHG1 | 36.08 | 3 | 0 | 3 | 0 |
| P49750_YLPM1_HUMAN | YLPM1 | 219.85 | 3 | 0 | 3 | 0 |
| P50991_TCPD_HUMAN | CCT4 | 57.89 | 3 | 0 | 3 | 0 |
| P51532_SMCA4_HUMAN | SMARCA4 | 184.53 | 3 | 0 | 3 | 0 |
| Q04917_1433F_HUMAN | YWHAH | 28.2 | 3 | 0 | 3 | 0 |
| Q13867_BLMH_HUMAN | BLMH | 52.53 | 3 | 0 | 3 | 0 |
| Q15773_MLF2_HUMAN | MLF2 | 28.13 | 3 | 0 | 3 | 0 |
| Q56A73_SPIN4_HUMAN | SPIN4 | 28.64 | 3 | 0 | 3 | 0 |
| Q7KZ85_SPT6H_HUMAN | SUPT6H | 198.95 | 3 | 0 | 3 | 0 |
| Q8N684_CPSF7_HUMAN | CPSF7 | 52.02 | 3 | 0 | 3 | 0 |
| Q8NI27_THOC2_HUMAN | THOC2 | 182.66 | 3 | 0 | 3 | 0 |
| Q8WVV4_POF1B_HUMAN | POF1B | 68.02 | 3 | 0 | 3 | 0 |
| Q9UNM6_PSD13_HUMAN | PSMD13 | 42.92 | 3 | 0 | 3 | 0 |
| Q6PCD5_RFWD3_HUMAN | RFWD3 | 85.04 | 1 | 0 | 3 | 0 |
| O00487_PSDE_HUMAN | PSMD14 | 34.55 | 2 | 0 | 2 | 0 |
| O43684_BUB3_HUMAN | BUB3 | 37.13 | 2 | 0 | 2 | 0 |
| O75223_GGCT_HUMAN | GGCT | 20.99 | 2 | 0 | 2 | 0 |
| O95757_HS74L_HUMAN | HSPA4L | 94.45 | 2 | 0 | 2 | 0 |
| O96019_ACL6A_HUMAN | ACTL6A | 47.43 | 2 | 0 | 2 | 0 |
| P01009_A1AT_HUMAN | SERPINA1 | 46.71 | 2 | 0 | 2 | 0 |
| P02787_TRFE_HUMAN | TF | 77.01 | 2 | 0 | 2 | 0 |
| P05091_ALDH2_HUMAN | ALDH2 | 56.35 | 2 | 0 | 2 | 0 |
| P07954_FUMH_HUMAN | FH | 54.6 | 2 | 0 | 2 | 0 |
| P09972_ALDOC_HUMAN | ALDOC | 39.43 | 2 | 0 | 2 | 0 |
| P14735_IDE_HUMAN | IDE | 117.89 | 2 | 0 | 2 | 0 |
| P16152_CBR1_HUMAN | CBR1 | 30.36 | 2 | 0 | 2 | 0 |
| P21796_VDAC1_HUMAN | VDAC1 | 30.75 | 2 | 0 | 2 | 0 |
| P25311_ZA2G_HUMAN | AZGP1 | 34.24 | 2 | 0 | 2 | 0 |
| P25788_PSA3_HUMAN | PSMA3 | 28.42 | 2 | 0 | 2 | 0 |
| P28066_PSA5_HUMAN | PSMA5 | 26.39 | 2 | 0 | 2 | 0 |
| P28070_PSB4_HUMAN | PSMB4 | 29.19 | 2 | 0 | 2 | 0 |
| P28074_PSB5_HUMAN | PSMB5 | 28.46 | 2 | 0 | 2 | 0 |
| P30040_ERP29_HUMAN | ERP29 | 28.98 | 2 | 0 | 2 | 0 |
| P31483_TIA1_HUMAN | TIA1 | 42.94 | 2 | 0 | 2 | 0 |
| P33240_CSTF2_HUMAN | CSTF2 | 60.92 | 2 | 0 | 2 | 0 |
| P35998_PRS7_HUMAN | PSMC2 | 48.6 | 2 | 0 | 2 | 0 |
| P42765_THIM_HUMAN | ACAA2 | 41.9 | 2 | 0 | 2 | 0 |
| P45974_UBP5_HUMAN | USP5 | 95.73 | 2 | 0 | 2 | 0 |
| P48047_ATPO_HUMAN | ATP5O | 23.26 | 2 | 0 | 2 | 0 |
| P49756_RBM25_HUMAN | RBM25 | 100.12 | 2 | 0 | 2 | 0 |
| P51610_HCFC1_HUMAN | HCFC1 | 208.6 | 2 | 0 | 2 | 0 |
| P52756_RBM5_HUMAN | RBM5 | 92.1 | 2 | 0 | 2 | 0 |
| P55786_PSA_HUMAN | NPEPPS | 103.21 | 2 | 0 | 2 | 0 |
| P78347_GTF2I_HUMAN | GTF2I | 112.35 | 2 | 0 | 2 | 0 |
| Q01085_TIAR_HUMAN | TIAL1 | 41.56 | 2 | 0 | 2 | 0 |
| Q06323_PSME1_HUMAN | PSME1 | 28.71 | 2 | 0 | 2 | 0 |
| Q08J23_NSUN2_HUMAN | NSUN2 | 86.42 | 2 | 0 | 2 | 0 |
| Q10570_CPSF1_HUMAN | CPSF1 | 160.78 | 2 | 0 | 2 | 0 |
| Q13200_PSMD2_HUMAN | PSMD2 | 100.14 | 2 | 0 | 2 | 0 |
| Q16610_ECM1_HUMAN | ECM1 | 60.64 | 2 | 0 | 2 | 0 |
| Q16836_HCDH_HUMAN | HADH | 34.27 | 2 | 0 | 2 | 0 |
| Q53RT3_APRV1_HUMAN | ASPRV1 | 36.97 | 2 | 0 | 2 | 0 |
| Q92769_HDAC2_HUMAN | HDAC2 | 55.33 | 2 | 0 | 2 | 0 |
| Q92797_SYMPK_HUMAN | SYMPK | 141.06 | 2 | 0 | 2 | 0 |
| Q92820_GGH_HUMAN | GGH | 35.94 | 2 | 0 | 2 | 0 |
| Q99623_PHB2_HUMAN | PHB2 | 33.28 | 2 | 0 | 2 | 0 |
| Q9BT78_CSN4_HUMAN | COPS4 | 46.24 | 2 | 0 | 2 | 0 |
| Q9HAZ1_CLK4_HUMAN | CLK4 | 57.46 | 2 | 0 | 2 | 0 |
| Q9Y230_RUVB2_HUMAN | RUVBL2 | 51.12 | 2 | 0 | 2 | 0 |
| P01834_IGKC_HUMAN | IGKC | 11.6 | 1 | 0 | 2 | 0 |
| Q86TJ2_TAD2B_HUMAN | TADA2B | 48.44 | 1 | 0 | 2 | 0 |
| A0A0A0MR66_A0A0A0MR66_HUMAN | RBM10 | 110.3 | 1 | 0 | 1 | 0 |
| B2RXH4_BTBDI_HUMAN | BTBD18 | 77.88 | 1 | 0 | 1 | 0 |
| F8VUG4_F8VUG4_HUMAN | MAP3K12 | 43.48 | 1 | 0 | 1 | 0 |
| IGHG_RABIT | | 35.38 | 1 | 0 | 1 | 0 |
| J3QR62_J3QR62_HUMAN | DDX5 | 5.48 | 1 | 0 | 1 | 0 |
| O00268_TAF4_HUMAN | TAF4 | 110.05 | 1 | 0 | 1 | 0 |
| O15355_PPM1G_HUMAN | PPM1G | 59.23 | 1 | 0 | 1 | 0 |
| O60437_PEPL_HUMAN | PPL | 204.62 | 1 | 0 | 1 | 0 |
| O60911_CATL2_HUMAN | CTSV | 37.31 | 1 | 0 | 1 | 0 |
| O75131_CPNE3_HUMAN | CPNE3 | 60.09 | 1 | 0 | 1 | 0 |
| O75635_SPB7_HUMAN | SERPINB7 | 42.88 | 1 | 0 | 1 | 0 |
| O75934_SPF27_HUMAN | BCAS2 | 26.11 | 1 | 0 | 1 | 0 |
| O75964_ATP5L_HUMAN | ATP5L | 11.42 | 1 | 0 | 1 | 0 |
| O95433_AHSA1_HUMAN | AHSA1 | 38.25 | 1 | 0 | 1 | 0 |
| O96008_TOM40_HUMAN | TOMM40 | 37.87 | 1 | 0 | 1 | 0 |
| P02671_FIBA_HUMAN | FGA | 94.91 | 1 | 0 | 1 | 0 |
| P02751_FINC_HUMAN | FN1 | 262.46 | 1 | 0 | 1 | 0 |
| P04040_CATA_HUMAN | CAT | 59.72 | 1 | 0 | 1 | 0 |
| P04844_RPN2_HUMAN | RPN2 | 69.24 | 1 | 0 | 1 | 0 |
| P05067_A4_HUMAN | APP | 86.89 | 1 | 0 | 1 | 0 |
| P07384_CAN1_HUMAN | CAPN1 | 81.84 | 1 | 0 | 1 | 0 |
| P08133_ANXA6_HUMAN | ANXA6 | 75.83 | 1 | 0 | 1 | 0 |
| P10155_RO60_HUMAN | TROVE2 | 60.63 | 1 | 0 | 1 | 0 |
| P12081_SYHC_HUMAN | HARS | 57.37 | 1 | 0 | 1 | 0 |
| P13489_RINI_HUMAN | RNH1 | 49.94 | 1 | 0 | 1 | 0 |
| P13667_PDIA4_HUMAN | PDIA4 | 72.89 | 1 | 0 | 1 | 0 |
| P14406_CX7A2_HUMAN | COX7A2 | 9.39 | 1 | 0 | 1 | 0 |
| P19404_NDUV2_HUMAN | NDUFV2 | 27.37 | 1 | 0 | 1 | 0 |
| P21802_FGFR2_HUMAN | FGFR2 | 91.97 | 1 | 0 | 1 | 0 |
| P25786_PSA1_HUMAN | PSMA1 | 29.54 | 1 | 0 | 1 | 0 |
| P27695_APEX1_HUMAN | APEX1 | 35.53 | 1 | 0 | 1 | 0 |
| P27797_CALR_HUMAN | CALR | 48.11 | 1 | 0 | 1 | 0 |
| P31949_S10AB_HUMAN | S100A11 | 11.73 | 1 | 0 | 1 | 0 |
| P35579_MYH9_HUMAN | MYH9 | 226.39 | 1 | 0 | 1 | 0 |
| P35580_MYH10_HUMAN | MYH10 | 228.86 | 1 | 0 | 1 | 0 |
| P38117_ETFB_HUMAN | ETFB | 27.83 | 1 | 0 | 1 | 0 |
| P39656_OST48_HUMAN | DDOST | 50.77 | 1 | 0 | 1 | 0 |
| P40937_RFC5_HUMAN | RFC5 | 38.47 | 1 | 0 | 1 | 0 |
| P41223_BUD31_HUMAN | BUD31 | 16.99 | 1 | 0 | 1 | 0 |
| P43487_RANG_HUMAN | RANBP1 | 23.3 | 1 | 0 | 1 | 0 |
| P45973_CBX5_HUMAN | CBX5 | 22.21 | 1 | 0 | 1 | 0 |
| P46060_RAGP1_HUMAN | RANGAP1 | 63.5 | 1 | 0 | 1 | 0 |
| P46940_IQGA1_HUMAN | IQGAP1 | 189.13 | 1 | 0 | 1 | 0 |
| P48556_PSMD8_HUMAN | PSMD8 | 39.59 | 1 | 0 | 1 | 0 |
| P49321_NASP_HUMAN | NASP | 85.19 | 1 | 0 | 1 | 0 |
| P49589_SYCC_HUMAN | CARS | 85.42 | 1 | 0 | 1 | 0 |
| P49721_PSB2_HUMAN | PSMB2 | 22.82 | 1 | 0 | 1 | 0 |
| P50454_SERPH_HUMAN | SERPINH1 | 46.41 | 1 | 0 | 1 | 0 |
| P50502_F10A1_HUMAN | ST13 | 41.31 | 1 | 0 | 1 | 0 |
| P51531_SMCA2_HUMAN | SMARCA2 | 181.17 | 1 | 0 | 1 | 0 |
| P51587_BRCA2_HUMAN | BRCA2 | 383.99 | 1 | 0 | 1 | 0 |
| P58107_EPIPL_HUMAN | EPPK1 | 555.28 | 1 | 0 | 1 | 0 |
| P68402_PA1B2_HUMAN | PAFAH1B2 | 25.55 | 1 | 0 | 1 | 0 |
| P78559_MAP1A_HUMAN | MAP1A | 305.3 | 1 | 0 | 1 | 0 |
| Q00325_MPCP_HUMAN | SLC25A3 | 40.07 | 1 | 0 | 1 | 0 |
| Q01082_SPTB2_HUMAN | SPTBN1 | 274.44 | 1 | 0 | 1 | 0 |
| Q03393_PTPS_HUMAN | PTS | 16.38 | 1 | 0 | 1 | 0 |
| Q12824_SNF5_HUMAN | SMARCB1 | 44.11 | 1 | 0 | 1 | 0 |
| Q13098_CSN1_HUMAN | GPS1 | 55.5 | 1 | 0 | 1 | 0 |
| Q15102_PA1B3_HUMAN | PAFAH1B3 | 25.72 | 1 | 0 | 1 | 0 |
| Q15517_CDSN_HUMAN | CDSN | 51.49 | 1 | 0 | 1 | 0 |
| Q16555_DPYL2_HUMAN | DPYSL2 | 62.25 | 1 | 0 | 1 | 0 |
| Q16891_MIC60_HUMAN | IMMT | 83.63 | 1 | 0 | 1 | 0 |
| Q29RF7_PDS5A_HUMAN | PDS5A | 150.73 | 1 | 0 | 1 | 0 |
| Q58FG0_HS905_HUMAN | HSP90AA5P | 38.71 | 1 | 0 | 1 | 0 |
| Q5T200_ZC3HD_HUMAN | ZC3H13 | 196.52 | 1 | 0 | 1 | 0 |
| Q5T750_XP32_HUMAN | XP32 | 26.22 | 1 | 0 | 1 | 0 |
| Q5UIP0_RIF1_HUMAN | RIF1 | 274.29 | 1 | 0 | 1 | 0 |
| Q6NZY4_ZCHC8_HUMAN | ZCCHC8 | 78.53 | 1 | 0 | 1 | 0 |
| Q6UXN9_WDR82_HUMAN | WDR82 | 35.06 | 1 | 0 | 1 | 0 |
| Q6ZMN7_PZRN4_HUMAN | PDZRN4 | 117.03 | 1 | 0 | 1 | 0 |
| Q6ZQQ6_WDR87_HUMAN | WDR87 | 332.97 | 1 | 0 | 1 | 0 |
| Q7RTV0_PHF5A_HUMAN | PHF5A | 12.4 | 1 | 0 | 1 | 0 |
| Q86W42_THOC6_HUMAN | THOC6 | 37.51 | 1 | 0 | 1 | 0 |
| Q8TEA8_DTD1_HUMAN | DTD1 | 23.41 | 1 | 0 | 1 | 0 |
| Q8TEQ8_PIGO_HUMAN | PIGO | 118.62 | 1 | 0 | 1 | 0 |
| Q8WUM4_PDC6I_HUMAN | PDCD6IP | 95.96 | 1 | 0 | 1 | 0 |
| Q8WXA9_SREK1_HUMAN | SREK1 | 59.35 | 1 | 0 | 1 | 0 |
| Q92541_RTF1_HUMAN | RTF1 | 80.26 | 1 | 0 | 1 | 0 |
| Q92734_TFG_HUMAN | TFG | 43.42 | 1 | 0 | 1 | 0 |
| Q92973_TNPO1_HUMAN | TNPO1 | 102.29 | 1 | 0 | 1 | 0 |
| Q93009_UBP7_HUMAN | USP7 | 128.22 | 1 | 0 | 1 | 0 |
| Q96EK9_KTI12_HUMAN | KTI12 | 38.59 | 1 | 0 | 1 | 0 |
| Q96FQ6_S10AG_HUMAN | S100A16 | 11.79 | 1 | 0 | 1 | 0 |
| Q99627_CSN8_HUMAN | COPS8 | 23.21 | 1 | 0 | 1 | 0 |
| Q9BZZ5_API5_HUMAN | API5 | 58.97 | 1 | 0 | 1 | 0 |
| Q9HAV4_XPO5_HUMAN | XPO5 | 136.22 | 1 | 0 | 1 | 0 |
| Q9HB07_MYG1_HUMAN | C12orf10 | 42.42 | 1 | 0 | 1 | 0 |
| Q9HCY8_S10AE_HUMAN | S100A14 | 11.65 | 1 | 0 | 1 | 0 |
| Q9NS84_CHST7_HUMAN | CHST7 | 54.23 | 1 | 0 | 1 | 0 |
| Q9NTK5_OLA1_HUMAN | OLA1 | 44.72 | 1 | 0 | 1 | 0 |
| Q9NVV2_CS073_HUMAN | C19orf73 | 13.74 | 1 | 0 | 1 | 0 |
| Q9P2F9_ZN319_HUMAN | ZNF319 | 65.5 | 1 | 0 | 1 | 0 |
| Q9UBB4_ATX10_HUMAN | ATXN10 | 53.45 | 1 | 0 | 1 | 0 |
| Q9UHD8_SEPT9_HUMAN | “SEPT9” | 65.36 | 1 | 0 | 1 | 0 |
| Q9UHV7_MED13_HUMAN | MED13 | 239.14 | 1 | 0 | 1 | 0 |
| Q9UHV9_PFD2_HUMAN | PFDN2 | 16.64 | 1 | 0 | 1 | 0 |
| Q9UNN5_FAF1_HUMAN | FAF1 | 73.91 | 1 | 0 | 1 | 0 |
| Q9Y2H1_ST38L_HUMAN | STK38L | 53.97 | 1 | 0 | 1 | 0 |
| Q9Y2Z0_SUGT1_HUMAN | SUGT1 | 41 | 1 | 0 | 1 | 0 |
| Q9Y337_KLK5_HUMAN | KLK5 | 32 | 1 | 0 | 1 | 0 |
| Q9Y383_LC7L2_HUMAN | LUC7L2 | 46.49 | 1 | 0 | 1 | 0 |
| Q9Y5L4_TIM13_HUMAN | TIMM13 | 10.49 | 1 | 0 | 1 | 0 |
| Q13619_CUL4A_HUMAN | CUL4A | 87.62 | 11 | 1 | 11 | 1 |
| P07355_ANXA2_HUMAN | ANXA2 | 38.58 | 10 | 1 | 11 | 1 |
| Q16531_DDB1_HUMAN | DDB1 | 126.89 | 46 | 4 | 52 | 5 |
| P07237_PDIA1_HUMAN | P4HB | 57.08 | 7 | 1 | 7 | 1 |
| Q5JUX0_SPIN3_HUMAN | SPIN3 | 29.19 | 10 | 2 | 10 | 2 |
| P23528_COF1_HUMAN | CFL1 | 18.49 | 5 | 1 | 5 | 1 |
| Q92499_DDX1_HUMAN | DDX1 | 82.38 | 5 | 1 | 5 | 1 |
| Q99460_PSMD1_HUMAN | PSMD1 | 105.77 | 5 | 1 | 5 | 1 |
| P31150_GDIA_HUMAN | GDI1 | 50.55 | 8 | 2 | 8 | 2 |
| P62258_1433E_HUMAN | YWHAE | 29.16 | 7 | 2 | 8 | 2 |
| P02545_LMNA_HUMAN | LMNA | 74.09 | 4 | 1 | 4 | 1 |
| P62333_PRS10_HUMAN | PSMC6 | 44.15 | 3 | 1 | 4 | 1 |
| Q9UHX1_PUF60_HUMAN | PUF60 | 59.84 | 16 | 6 | 20 | 6 |
| Q9Y657_SPIN1_HUMAN | SPIN1 | 29.58 | 9 | 3 | 10 | 3 |
| P62191_PRS4_HUMAN | PSMC1 | 49.15 | 6 | 2 | 6 | 2 |
| Q99865_SPI2A_HUMAN | SPIN2A | 29.17 | 5 | 2 | 6 | 2 |
| O15042_SR140_HUMAN | U2SURP | 118.22 | 3 | 1 | 3 | 1 |
| O75400_PR40A_HUMAN | PRPF40A | 108.74 | 3 | 1 | 3 | 1 |
| P10599_THIO_HUMAN | TXN | 11.73 | 3 | 1 | 3 | 1 |
| P20618_PSB1_HUMAN | PSMB1 | 26.47 | 3 | 1 | 3 | 1 |
| P24928_RPB1_HUMAN | POLR2A | 217.04 | 3 | 1 | 3 | 1 |
| P25787_PSA2_HUMAN | PSMA2 | 25.88 | 3 | 1 | 3 | 1 |
| P25789_PSA4_HUMAN | PSMA4 | 29.47 | 3 | 1 | 3 | 1 |
| P37837_TALDO_HUMAN | TALDO1 | 37.52 | 3 | 1 | 3 | 1 |
| P49368_TCPG_HUMAN | CCT3 | 60.5 | 3 | 1 | 3 | 1 |
| Q13242_SRSF9_HUMAN | SRSF9 | 25.53 | 3 | 1 | 3 | 1 |
| Q14247_SRC8_HUMAN | CTTN | 61.55 | 3 | 1 | 3 | 1 |
| Q16630_CPSF6_HUMAN | CPSF6 | 59.17 | 3 | 1 | 3 | 1 |
| Q8TAA3_PSA7L_HUMAN | PSMA8 | 28.51 | 3 | 1 | 3 | 1 |
| Q9Y3I0_RTCB_HUMAN | RTCB | 55.17 | 3 | 1 | 3 | 1 |
| P41250_SYG_HUMAN | GARS | 83.11 | 8 | 3 | 8 | 3 |
| P32119_PRDX2_HUMAN | PRDX2 | 21.88 | 7 | 3 | 8 | 3 |
| P54577_SYYC_HUMAN | YARS | 59.11 | 5 | 2 | 5 | 2 |
| Q9BXP5_SRRT_HUMAN | SRRT | 100.6 | 5 | 2 | 5 | 2 |
| O43447_PPIH_HUMAN | PPIH | 19.2 | 3 | 2 | 5 | 2 |
| Q9Y2W1_TR150_HUMAN | THRAP3 | 108.6 | 35 | 18 | 50 | 21 |
| Q15208_STK38_HUMAN | STK38 | 54.16 | 15 | 7 | 19 | 8 |
| P09493_TPM1_HUMAN | TPM1 | 32.69 | 6 | 3 | 7 | 3 |
| P27694_RFA1_HUMAN | RPA1 | 68.1 | 5 | 3 | 7 | 3 |
| Q9NYF8_BCLF1_HUMAN | BCLAF1 | 106.06 | 33 | 15 | 41 | 18 |
| Q14152_EIF3A_HUMAN | EIF3A | 166.47 | 35 | 20 | 40 | 20 |
| P22314_UBA1_HUMAN | UBA1 | 117.77 | 9 | 5 | 10 | 5 |
| P17980_PRS6A_HUMAN | PSMC3 | 49.17 | 6 | 3 | 6 | 3 |
| P23284_PPIB_HUMAN | PPIB | 23.73 | 5 | 3 | 6 | 3 |
| P46821_MAP1B_HUMAN | MAP1B | 270.47 | 4 | 2 | 4 | 2 |
| Q06830_PRDX1_HUMAN | PRDX1 | 22.1 | 4 | 2 | 4 | 2 |
| Q86VP6_CAND1_HUMAN | CAND1 | 136.29 | 4 | 2 | 4 | 2 |
| O14979_HNRDL_HUMAN | HNRNPDL | 46.41 | 2 | 1 | 2 | 1 |
| O43809_CPSF5_HUMAN | NUDT21 | 26.21 | 2 | 1 | 2 | 1 |
| O60264_SMCA5_HUMAN | SMARCA5 | 121.83 | 2 | 1 | 2 | 1 |
| P00492_HPRT_HUMAN | HPRT1 | 24.56 | 2 | 1 | 2 | 1 |
| P0DME0_SETLP_HUMAN | SETSIP | 34.86 | 2 | 1 | 2 | 1 |
| P12277_KCRB_HUMAN | CKB | 42.62 | 2 | 1 | 2 | 1 |
| P26038_MOES_HUMAN | MSN | 67.78 | 2 | 1 | 2 | 1 |
| P30048_PRDX3_HUMAN | PRDX3 | 27.68 | 2 | 1 | 2 | 1 |
| P34896_GLYC_HUMAN | SHMT1 | 53.05 | 2 | 1 | 2 | 1 |
| P35813_PPM1A_HUMAN | PPM1A | 42.42 | 2 | 1 | 2 | 1 |
| P39748_FEN1_HUMAN | FEN1 | 42.57 | 2 | 1 | 2 | 1 |
| P40925_MDHC_HUMAN | MDH1 | 36.4 | 2 | 1 | 2 | 1 |
| P43363_MAGAA_HUMAN | MAGEA10 | 40.75 | 2 | 1 | 2 | 1 |
| P49588_SYAC_HUMAN | AARS | 106.74 | 2 | 1 | 2 | 1 |
| P49915_GUAA_HUMAN | GMPS | 76.67 | 2 | 1 | 2 | 1 |
| P51149_RAB7A_HUMAN | RAB7A | 23.47 | 2 | 1 | 2 | 1 |
| P59998_ARPC4_HUMAN | ARPC4 | 19.65 | 2 | 1 | 2 | 1 |
| P62136_PP1A_HUMAN | PPP1CA | 37.49 | 2 | 1 | 2 | 1 |
| P62266_RS23_HUMAN | RPS23 | 15.8 | 2 | 1 | 2 | 1 |
| Q13547_HDAC1_HUMAN | HDAC1 | 55.07 | 2 | 1 | 2 | 1 |
| Q15287_RNPS1_HUMAN | RNPS1 | 34.19 | 2 | 1 | 2 | 1 |
| Q8IXT5_RB12B_HUMAN | RBM12B | 118.03 | 2 | 1 | 2 | 1 |
| Q8WXF1_PSPC1_HUMAN | PSPC1 | 58.71 | 2 | 1 | 2 | 1 |
| Q9ULX6_AKP8L_HUMAN | AKAP8L | 71.6 | 2 | 1 | 2 | 1 |
| Q9UQE7_SMC3_HUMAN | SMC3 | 141.45 | 2 | 1 | 2 | 1 |
| Q9Y4L1_HYOU1_HUMAN | HYOU1 | 111.27 | 2 | 1 | 2 | 1 |
| O60292_SI1L3_HUMAN | SIPA1L3 | 194.49 | 1 | 1 | 2 | 1 |
| Q9NWB1_RFOX1_HUMAN | RBFOX1 | 42.76 | 1 | 1 | 2 | 1 |
| Q9Y333_LSM2_HUMAN | LSM2 | 10.83 | 1 | 1 | 2 | 1 |
| P09874_PARP1_HUMAN | PARP1 | 113.01 | 47 | 30 | 54 | 31 |
| P78527_PRKDC_HUMAN | PRKDC | 468.79 | 25 | 16 | 27 | 16 |
| O43242_PSMD3_HUMAN | PSMD3 | 60.94 | 5 | 3 | 5 | 3 |
| P15531_NDKA_HUMAN | NME1 | 17.14 | 5 | 3 | 5 | 3 |
| P62081_RS7_HUMAN | RPS7 | 22.11 | 5 | 3 | 5 | 3 |
| P63104_1433Z_HUMAN | YWHAZ | 27.73 | 5 | 3 | 5 | 3 |
| P06733_ENOA_HUMAN | ENO1 | 47.14 | 25 | 17 | 28 | 17 |
| Q9HCD5_NCOA5_HUMAN | NCOA5 | 65.5 | 8 | 5 | 8 | 5 |
| O43852_CALU_HUMAN | CALU | 37.08 | 12 | 8 | 12 | 8 |
| P07737_PROF1_HUMAN | PFN1 | 15.04 | 6 | 4 | 6 | 4 |
| P12268_IMDH2_HUMAN | IMPDH2 | 55.77 | 6 | 4 | 6 | 4 |
| P48643_TCPE_HUMAN | CCT5 | 59.63 | 6 | 4 | 6 | 4 |
| A8MWD9_RUXGL_HUMAN | SNRPGP15 | 8.54 | 3 | 2 | 3 | 2 |
| O14980_XPO1_HUMAN | XPO1 | 123.31 | 3 | 2 | 3 | 2 |
| P06744_G6PI_HUMAN | GPI | 63.11 | 3 | 2 | 3 | 2 |
| P13804_ETFA_HUMAN | ETFA | 35.06 | 3 | 2 | 3 | 2 |
| P17174_AATC_HUMAN | GOT1 | 46.22 | 3 | 2 | 3 | 2 |
| P18669_PGAM1_HUMAN | PGAM1 | 28.79 | 3 | 2 | 3 | 2 |
| P27348_1433T_HUMAN | YWHAQ | 27.75 | 3 | 2 | 3 | 2 |
| P60228_EIF3E_HUMAN | EIF3E | 52.19 | 3 | 2 | 3 | 2 |
| P84077_ARF1_HUMAN | ARF1 | 20.68 | 3 | 2 | 3 | 2 |
| Q14974_IMB1_HUMAN | KPNB1 | 97.11 | 3 | 2 | 3 | 2 |
| Q15293_RCN1_HUMAN | RCN1 | 38.87 | 3 | 2 | 3 | 2 |
| Q16629_SRSF7_HUMAN | SRSF7 | 27.35 | 3 | 2 | 3 | 2 |
| Q8NEY8_PPHLN_HUMAN | PPHLN1 | 52.71 | 3 | 2 | 3 | 2 |
| P46783_RS10_HUMAN | RPS10 | 18.89 | 2 | 2 | 3 | 2 |
| Q9BUQ8_DDX23_HUMAN | DDX23 | 95.52 | 2 | 2 | 3 | 2 |
| P08238_HS90B_HUMAN | HSP90AB1 | 83.21 | 7 | 5 | 7 | 5 |
| Q07955_SRSF1_HUMAN | SRSF1 | 27.73 | 7 | 4 | 7 | 5 |
| P98175_RBM10_HUMAN | RBM10 | 103.47 | 40 | 29 | 44 | 32 |
| Q8N163_CCAR2_HUMAN | CCAR2 | 102.84 | 8 | 6 | 8 | 6 |
| P08865_RSSA_HUMAN | RPSA | 32.83 | 4 | 3 | 4 | 3 |
| P31153_METK2_HUMAN | MAT2A | 43.63 | 4 | 3 | 4 | 3 |
| P40227_TCPZ_HUMAN | CCT6A | 57.99 | 4 | 3 | 4 | 3 |
| Q00610_CLH1_HUMAN | CLTC | 191.49 | 4 | 3 | 4 | 3 |
| Q14103_HNRPD_HUMAN | HNRNPD | 38.41 | 4 | 3 | 4 | 3 |
| O00148_DX39A_HUMAN | DDX39A | 49.1 | 3 | 3 | 4 | 3 |
| P62987_RL40_HUMAN | UBA52 | 14.72 | 3 | 3 | 4 | 3 |
| P23588_IF4B_HUMAN | EIF4B | 69.11 | 26 | 27 | 58 | 44 |
| P13010_XRCC5_HUMAN | XRCC5 | 82.65 | 16 | 13 | 17 | 13 |
| P14625_ENPL_HUMAN | HSP90B1 | 92.41 | 13 | 9 | 13 | 10 |
| O00422_SAP18_HUMAN | SAP18 | 17.55 | 8 | 5 | 9 | 7 |
| P06748_NPM_HUMAN | NPM1 | 32.55 | 5 | 4 | 5 | 4 |
| P40926_MDHM_HUMAN | MDH2 | 35.48 | 5 | 4 | 5 | 4 |
| P62269_RS18_HUMAN | RPS18 | 17.71 | 5 | 4 | 5 | 4 |
| G3XAC6_G3XAC6_HUMAN | RBM39 | 47.99 | 1 | 1 | 5 | 4 |
| P14618_KPYM_HUMAN | PKM | 57.9 | 12 | 10 | 12 | 10 |
| P12956_XRCC6_HUMAN | XRCC6 | 69.8 | 17 | 16 | 20 | 17 |
| P29401_TKT_HUMAN | TKT | 67.83 | 7 | 5 | 8 | 7 |
| Q13263_TIF1B_HUMAN | TRIM28 | 88.49 | 9 | 9 | 10 | 9 |
| P25205_MCM3_HUMAN | MCM3 | 90.92 | 11 | 11 | 12 | 11 |
| P62805_H4_HUMAN | HIST1H4A | 11.36 | 10 | 9 | 12 | 11 |
| P10809_CH60_HUMAN | HSPD1 | 61.02 | 20 | 19 | 21 | 20 |
| P78332_RBM6_HUMAN | RBM6 | 128.57 | 21 | 21 | 23 | 23 |
| Q12905_ILF2_HUMAN | ILF2 | 43.04 | 16 | 14 | 17 | 17 |
| P68104_EF1A1_HUMAN | EEF1A1 | 50.11 | 12 | 13 | 13 | 13 |
| P00558_PGK1_HUMAN | PGK1 | 44.59 | 10 | 10 | 11 | 11 |
| P09661_RU2A_HUMAN | SNRPA1 | 28.4 | 10 | 11 | 11 | 11 |
| Q8WUA2_PPIL4_HUMAN | PPIL4 | 57.19 | 10 | 9 | 11 | 11 |
| Q9UKM9_RALY_HUMAN | RALY | 32.44 | 10 | 9 | 10 | 10 |
| P55072_TERA_HUMAN | VCP | 89.27 | 9 | 9 | 10 | 10 |
| Q32P51_RA1L2_HUMAN | HNRNPA1L2 | 34.2 | 9 | 9 | 10 | 10 |
| P41219_PERI_HUMAN | PRPH | 53.62 | 8 | 8 | 8 | 8 |
| Q58FF7_H90B3_HUMAN | HSP90AB3P | 68.28 | 7 | 7 | 7 | 7 |
| P00338_LDHA_HUMAN | LDHA | 36.67 | 6 | 6 | 6 | 6 |
| P62701_RS4X_HUMAN | RPS4X | 29.58 | 6 | 6 | 6 | 6 |
| P62995_TRA2B_HUMAN | TRA2B | 33.65 | 6 | 6 | 6 | 6 |
| Q9UQ80_PA2G4_HUMAN | PA2G4 | 43.76 | 6 | 5 | 6 | 6 |
| B4DY08_B4DY08_HUMAN | HNRNPC | 31.95 | 5 | 5 | 6 | 6 |
| Q96QV6_H2A1A_HUMAN | HIST1H2AA | 14.22 | 4 | 4 | 6 | 6 |
| P04075_ALDOA_HUMAN | ALDOA | 39.4 | 5 | 5 | 5 | 5 |
| P11586_C1TC_HUMAN | MTHFD1 | 101.5 | 5 | 5 | 5 | 5 |
| P62826_RAN_HUMAN | RAN | 24.41 | 5 | 5 | 5 | 5 |
| Q9H307_PININ_HUMAN | PNN | 81.56 | 5 | 5 | 5 | 5 |
| Q9Y5B9_SP16H_HUMAN | SUPT16H | 119.84 | 5 | 4 | 5 | 5 |
| P41252_SYIC_HUMAN | IARS | 144.41 | 4 | 3 | 4 | 4 |
| Q9Y265_RUVB1_HUMAN | RUVBL1 | 50.2 | 4 | 4 | 4 | 4 |
| KV2A7_MOUSE | | 12.27 | 3 | 3 | 4 | 4 |
| P56537_IF6_HUMAN | EIF6 | 26.58 | 3 | 4 | 4 | 4 |
| O75494_SRS10_HUMAN | SRSF10 | 31.28 | 3 | 3 | 3 | 3 |
| P00505_AATM_HUMAN | GOT2 | 47.49 | 3 | 3 | 3 | 3 |
| P04792_HSPB1_HUMAN | HSPB1 | 22.77 | 3 | 3 | 3 | 3 |
| P05141_ADT2_HUMAN | SLC25A5 | 32.83 | 3 | 3 | 3 | 3 |
| P05455_LA_HUMAN | SSB | 46.81 | 3 | 3 | 3 | 3 |
| P08243_ASNS_HUMAN | ASNS | 64.33 | 3 | 3 | 3 | 3 |
| P45880_VDAC2_HUMAN | VDAC2 | 31.55 | 3 | 3 | 3 | 3 |
| P49411_EFTU_HUMAN | TUFM | 49.51 | 3 | 3 | 3 | 3 |
| P62318_SMD3_HUMAN | SNRPD3 | 13.91 | 3 | 3 | 3 | 3 |
| P63244_GBLP_HUMAN | GNB2L1 | 35.05 | 3 | 3 | 3 | 3 |
| Q99798_ACON_HUMAN | ACO2 | 85.37 | 3 | 3 | 3 | 3 |
| Q9Y617_SERC_HUMAN | PSAT1 | 40.4 | 3 | 3 | 3 | 3 |
| Q4LDE5_SVEP1_HUMAN | SVEP1 | 389.91 | 1 | 2 | 3 | 3 |
| A6NHL2_TBAL3_HUMAN | TUBAL3 | 49.88 | 2 | 2 | 2 | 2 |
| O15145_ARPC3_HUMAN | ARPC3 | 20.53 | 2 | 2 | 2 | 2 |
| P00367_DHE3_HUMAN | GLUD1 | 61.36 | 2 | 2 | 2 | 2 |
| P04908_H2A1B_HUMAN | HIST1H2AB | 14.13 | 2 | 2 | 2 | 2 |
| P0CW22_RS17L_HUMAN | RPS17L | 15.54 | 2 | 2 | 2 | 2 |
| P11388_TOP2A_HUMAN | TOP2A | 174.28 | 2 | 2 | 2 | 2 |
| P17661_DESM_HUMAN | DES | 53.5 | 2 | 2 | 2 | 2 |
| P24752_THIL_HUMAN | ACAT1 | 45.17 | 2 | 2 | 2 | 2 |
| P29692_EF1D_HUMAN | EEF1D | 31.1 | 2 | 2 | 2 | 2 |
| P35268_RL22_HUMAN | RPL22 | 14.78 | 2 | 2 | 2 | 2 |
| P42166_LAP2A_HUMAN | TMPO | 75.45 | 2 | 2 | 2 | 2 |
| P43686_PRS6B_HUMAN | PSMC4 | 47.34 | 2 | 2 | 2 | 2 |
| P49720_PSB3_HUMAN | PSMB3 | 22.93 | 2 | 2 | 2 | 2 |
| P49759_CLK1_HUMAN | CLK1 | 57.25 | 2 | 2 | 2 | 2 |
| P51571_SSRD_HUMAN | SSR4 | 18.99 | 2 | 2 | 2 | 2 |
| P61160_ARP2_HUMAN | ACTR2 | 44.73 | 2 | 2 | 2 | 2 |
| P62491_RB11A_HUMAN | RAB11A | 24.38 | 2 | 2 | 2 | 2 |
| P62807_H2B1C_HUMAN | HIST1H2BC | 13.9 | 2 | 2 | 2 | 2 |
| P62913_RL11_HUMAN | RPL11 | 20.24 | 2 | 2 | 2 | 2 |
| P84090_ERH_HUMAN | ERH | 12.25 | 2 | 2 | 2 | 2 |
| P84103_SRSF3_HUMAN | SRSF3 | 19.32 | 2 | 2 | 2 | 2 |
| Q14194_DPYL1_HUMAN | CRMP1 | 62.14 | 2 | 2 | 2 | 2 |
| Q14566_MCM6_HUMAN | MCM6 | 92.83 | 2 | 2 | 2 | 2 |
| Q53GS9_SNUT2_HUMAN | USP39 | 65.34 | 2 | 2 | 2 | 2 |
| Q71UM5_RS27L_HUMAN | RPS27L | 9.47 | 2 | 2 | 2 | 2 |
| Q9NQ39_RS10L_HUMAN | RPS10P5 | 20.11 | 2 | 2 | 2 | 2 |
| Q9UJZ1_STML2_HUMAN | STOML2 | 38.51 | 2 | 2 | 2 | 2 |
| C9JUF0_C9JUF0_HUMAN | EIF4A2 | 6.32 | 1 | 1 | 1 | 1 |
| D6RBZ0_D6RBZ0_HUMAN | HNRNPAB | 35.66 | 1 | 1 | 1 | 1 |
| E5RFV3_E5RFV3_HUMAN | SREK1 | 14.3 | 1 | 1 | 1 | 1 |
| E9PAV3_NACAM_HUMAN | NACA | 205.29 | 1 | 1 | 1 | 1 |
| H7BZJ3_H7BZJ3_HUMAN | PDIA3 | 13.51 | 1 | 1 | 1 | 1 |
| O00232_PSD12_HUMAN | PSMD12 | 52.87 | 1 | 1 | 1 | 1 |
| O00299_CLIC1_HUMAN | CLIC1 | 26.91 | 1 | 1 | 1 | 1 |
| O14556_G3PT_HUMAN | GAPDHS | 44.47 | 1 | 1 | 1 | 1 |
| O15347_HMGB3_HUMAN | HMGB3 | 22.97 | 1 | 1 | 1 | 1 |
| O15371_EIF3D_HUMAN | EIF3D | 63.93 | 1 | 1 | 1 | 1 |
| O75390_CISY_HUMAN | CS | 51.68 | 1 | 1 | 1 | 1 |
| O95391_SLU7_HUMAN | SLU7 | 68.34 | 1 | 1 | 1 | 1 |
| P05198_IF2A_HUMAN | EIF2S1 | 36.09 | 1 | 1 | 1 | 1 |
| P08195_4F2_HUMAN | SLC3A2 | 67.95 | 1 | 1 | 1 | 1 |
| P09012_SNRPA_HUMAN | SNRPA | 31.26 | 1 | 1 | 1 | 1 |
| P11387_TOP1_HUMAN | TOP1 | 90.67 | 1 | 1 | 1 | 1 |
| P12273_PIP_HUMAN | PIP | 16.56 | 1 | 1 | 1 | 1 |
| P13473_LAMP2_HUMAN | LAMP2 | 44.93 | 1 | 1 | 1 | 1 |
| P14649_MYL6B_HUMAN | MYL6B | 22.75 | 1 | 1 | 1 | 1 |
| P22234_PUR6_HUMAN | PAICS | 47.05 | 1 | 1 | 1 | 1 |
| P28072_PSB6_HUMAN | PSMB6 | 25.34 | 1 | 1 | 1 | 1 |
| P31350_RIR2_HUMAN | RRM2 | 44.85 | 1 | 1 | 1 | 1 |
| P36957_ODO2_HUMAN | DLST | 48.72 | 1 | 1 | 1 | 1 |
| P42285_SK2L2_HUMAN | SKIV2L2 | 117.73 | 1 | 1 | 1 | 1 |
| P48444_COPD_HUMAN | ARCN1 | 57.17 | 1 | 1 | 1 | 1 |
| P49207_RL34_HUMAN | RPL34 | 13.28 | 1 | 1 | 1 | 1 |
| P50395_GDIB_HUMAN | GDI2 | 50.63 | 1 | 1 | 1 | 1 |
| P56385_ATP5I_HUMAN | ATP5I | 7.93 | 1 | 1 | 1 | 1 |
| P62310_LSM3_HUMAN | LSM3 | 11.84 | 1 | 1 | 1 | 1 |
| P62820_RAB1A_HUMAN | RAB1A | 22.66 | 1 | 1 | 1 | 1 |
| P62854_RS26_HUMAN | RPS26 | 13.01 | 1 | 1 | 1 | 1 |
| P68363_TBA1B_HUMAN | TUBA1B | 50.12 | 1 | 1 | 1 | 1 |
| P68371_TBB4B_HUMAN | TUBB4B | 49.8 | 1 | 1 | 1 | 1 |
| Q02880_TOP2B_HUMAN | TOP2B | 183.15 | 1 | 1 | 1 | 1 |
| Q05519_SRS11_HUMAN | SRSF11 | 53.51 | 1 | 1 | 1 | 1 |
| Q08170_SRSF4_HUMAN | SRSF4 | 56.65 | 1 | 1 | 1 | 1 |
| Q12873_CHD3_HUMAN | CHD3 | 226.45 | 1 | 1 | 1 | 1 |
| Q13011_ECH1_HUMAN | ECH1 | 35.79 | 1 | 1 | 1 | 1 |
| Q13247_SRSF6_HUMAN | SRSF6 | 39.56 | 1 | 1 | 1 | 1 |
| Q16637_SMN_HUMAN | SMN1 | 31.83 | 1 | 1 | 1 | 1 |
| Q5JNZ5_RS26L_HUMAN | RPS26P11 | 12.99 | 1 | 1 | 1 | 1 |
| Q8WWF6_DNJB3_HUMAN | DNAJB3 | 16.55 | 1 | 1 | 1 | 1 |
| Q92665_RT31_HUMAN | MRPS31 | 45.29 | 1 | 1 | 1 | 1 |
| Q96AE4_FUBP1_HUMAN | FUBP1 | 67.52 | 1 | 1 | 1 | 1 |
| Q99536_VAT1_HUMAN | VAT1 | 41.89 | 1 | 1 | 1 | 1 |
| Q9BWF3_RBM4_HUMAN | RBM4 | 40.29 | 1 | 1 | 1 | 1 |
| Q9P0M6_H2AW_HUMAN | H2AFY2 | 40.03 | 1 | 1 | 1 | 1 |
| Q9UKF6_CPSF3_HUMAN | CPSF3 | 77.44 | 1 | 1 | 1 | 1 |
| Q9Y224_CN166_HUMAN | C14orf166 | 28.05 | 1 | 1 | 1 | 1 |
| Q9Y285_SYFA_HUMAN | FARSA | 57.53 | 1 | 1 | 1 | 1 |
| Q14498_RBM39_HUMAN | RBM39 | 59.34 | 52 | 52 | 259 | 267 |
| Q15393_SF3B3_HUMAN | SF3B3 | 135.49 | 33 | 35 | 36 | 38 |
| P61978_HNRPK_HUMAN | HNRNPK | 50.94 | 15 | 15 | 15 | 16 |
| P04406_G3P_HUMAN | GAPDH | 36.03 | 11 | 10 | 14 | 15 |
| B7ZW38_HNRC3_HUMAN | HNRNPCL3 | 32.01 | 8 | 8 | 8 | 9 |
| P07195_LDHB_HUMAN | LDHB | 36.62 | 8 | 9 | 8 | 9 |
| Q8WWY3_PRP31_HUMAN | PRPF31 | 55.42 | 8 | 8 | 8 | 9 |
| P23396_RS3_HUMAN | RPS3 | 26.67 | 15 | 15 | 15 | 17 |
| Q6P2Q9_PRP8_HUMAN | PRPF8 | 273.43 | 67 | 78 | 74 | 84 |
| Q99459_CDC5L_HUMAN | CDC5L | 92.19 | 12 | 14 | 14 | 16 |
| P47756_CAPZB_HUMAN | CAPZB | 31.33 | 7 | 7 | 7 | 8 |
| Q15233_NONO_HUMAN | NONO | 54.2 | 7 | 8 | 7 | 8 |
| Q9BY77_PDIP3_HUMAN | POLDIP3 | 46.06 | 7 | 7 | 7 | 8 |
| Q14257_RCN2_HUMAN | RCN2 | 36.85 | 6 | 8 | 7 | 8 |
| Q00839_HNRPU_HUMAN | HNRNPU | 90.53 | 13 | 14 | 13 | 15 |
| Q9UMS4_PRP19_HUMAN | PRPF19 | 55.15 | 11 | 11 | 12 | 14 |
| P09651_ROA1_HUMAN | HNRNPA1 | 38.72 | 5 | 5 | 12 | 14 |
| O43175_SERA_HUMAN | PHGDH | 56.61 | 6 | 7 | 6 | 7 |
| P14678_RSMB_HUMAN | SNRPB | 24.59 | 6 | 6 | 6 | 7 |
| P54652_HSP72_HUMAN | HSPA2 | 69.98 | 6 | 7 | 6 | 7 |
| Q96A72_MGN2_HUMAN | MAGOHB | 17.26 | 5 | 7 | 6 | 7 |
| P51991_ROA3_HUMAN | HNRNPA3 | 39.57 | 10 | 12 | 11 | 13 |
| P22626_ROA2_HUMAN | HNRNPA2B1 | 37.41 | 15 | 15 | 16 | 19 |
| Q9Y2X3_NOP58_HUMAN | NOP58 | 59.54 | 14 | 16 | 15 | 18 |
| P23246_SFPQ_HUMAN | SFPQ | 76.1 | 5 | 6 | 5 | 6 |
| P23526_SAHH_HUMAN | AHCY | 47.69 | 5 | 6 | 5 | 6 |
| P62244_RS15A_HUMAN | RPS15A | 14.83 | 5 | 6 | 5 | 6 |
| Q15084_PDIA6_HUMAN | PDIA6 | 48.09 | 5 | 5 | 5 | 6 |
| Q15427_SF3B4_HUMAN | SF3B4 | 44.36 | 5 | 5 | 5 | 6 |
| Q09028_RBBP4_HUMAN | RBBP4 | 47.63 | 4 | 6 | 5 | 6 |
| Q08211_DHX9_HUMAN | DHX9 | 140.87 | 14 | 17 | 14 | 17 |
| O14744_ANM5_HUMAN | PRMT5 | 72.64 | 9 | 11 | 9 | 11 |
| O75533_SF3B1_HUMAN | SF3B1 | 145.74 | 43 | 53 | 45 | 56 |
| O43290_SNUT1_HUMAN | SART1 | 90.2 | 8 | 9 | 8 | 10 |
| Q58FF8_H90B2_HUMAN | HSP90AB2P | 44.32 | 7 | 10 | 8 | 10 |
| P04350_TBB4A_HUMAN | TUBB4A | 49.55 | 4 | 5 | 4 | 5 |
| P34897_GLYM_HUMAN | SHMT2 | 55.96 | 4 | 4 | 4 | 5 |
| P62304_RUXE_HUMAN | SNRPE | 10.8 | 4 | 5 | 4 | 5 |
| Q16695_H31T_HUMAN | HIST3H3 | 15.5 | 4 | 5 | 4 | 5 |
| Q86V81_THOC4_HUMAN | ALYREF | 26.87 | 4 | 4 | 4 | 5 |
| Q9Y3B4_SF3B6_HUMAN | SF3B6 | 14.58 | 4 | 5 | 4 | 5 |
| Q96PK6_RBM14_HUMAN | RBM14 | 69.45 | 3 | 5 | 4 | 5 |
| P06576_ATPB_HUMAN | ATP5B | 56.52 | 14 | 18 | 14 | 18 |
| P33993_MCM7_HUMAN | MCM7 | 81.26 | 7 | 9 | 7 | 9 |
| O43143_DHX15_HUMAN | DHX15 | 90.88 | 22 | 28 | 24 | 31 |
| Q13509_TBB3_HUMAN | TUBB3 | 50.4 | 8 | 10 | 10 | 13 |
| P38646_GRP75_HUMAN | HSPA9 | 73.63 | 12 | 15 | 13 | 17 |
| Q13435_SF3B2_HUMAN | SF3B2 | 100.16 | 26 | 32 | 27 | 36 |
| Q15029_U5S1_HUMAN | EFTUD2 | 109.37 | 23 | 32 | 24 | 32 |
| P19338_NUCL_HUMAN | NCL | 76.57 | 12 | 16 | 12 | 16 |
| P13639_EF2_HUMAN | EEF2 | 95.28 | 10 | 16 | 12 | 16 |
| P49736_MCM2_HUMAN | MCM2 | 101.83 | 9 | 12 | 9 | 12 |
| Q96A08_H2B1A_HUMAN | HIST1H2BA | 14.16 | 3 | 4 | 6 | 8 |
| P12004_PCNA_HUMAN | PCNA | 28.75 | 3 | 4 | 3 | 4 |
| P15311_EZRI_HUMAN | EZR | 69.37 | 3 | 4 | 3 | 4 |
| P27824_CALX_HUMAN | CANX | 67.53 | 3 | 4 | 3 | 4 |
| P30050_RL12_HUMAN | RPL12 | 17.81 | 3 | 4 | 3 | 4 |
| P55265_DSRAD_HUMAN | ADAR | 135.98 | 3 | 4 | 3 | 4 |
| P82979_SARNP_HUMAN | SARNP | 23.66 | 3 | 4 | 3 | 4 |
| Q07021_C1QBP_HUMAN | C1QBP | 31.34 | 3 | 4 | 3 | 4 |
| Q13148_TADBP_HUMAN | TARDBP | 44.71 | 3 | 4 | 3 | 4 |
| P34932_HSP74_HUMAN | HSPA4 | 94.27 | 2 | 4 | 3 | 4 |
| Q13595_TRA2A_HUMAN | TRA2A | 32.67 | 2 | 3 | 3 | 4 |
| O75643_U520_HUMAN | SNRNP200 | 244.35 | 47 | 63 | 49 | 66 |
| P14866_HNRPL_HUMAN | HNRNPL | 64.09 | 14 | 16 | 16 | 22 |
| Q13838_DX39B_HUMAN | DDX39B | 48.96 | 19 | 25 | 20 | 28 |
| P43243_MATR3_HUMAN | MATR3 | 94.56 | 14 | 19 | 15 | 21 |
| P07900_HS90A_HUMAN | HSP90AA1 | 84.61 | 10 | 13 | 10 | 14 |
| P31943_HNRH1_HUMAN | HNRNPH1 | 49.2 | 10 | 9 | 10 | 14 |
| P26599_PTBP1_HUMAN | PTBP1 | 57.19 | 9 | 12 | 10 | 14 |
| P20700_LMNB1_HUMAN | LMNB1 | 66.37 | 5 | 7 | 5 | 7 |
| Q86XP3_DDX42_HUMAN | DDX42 | 102.91 | 5 | 7 | 5 | 7 |
| Q9H4B7_TBB1_HUMAN | TUBB1 | 50.29 | 3 | 3 | 5 | 7 |
| Q15365_PCBP1_HUMAN | PCBP1 | 37.47 | 30 | 31 | 59 | 84 |
| Q15459_SF3A1_HUMAN | SF3A1 | 88.83 | 17 | 22 | 18 | 26 |
| Q15366_PCBP2_HUMAN | PCBP2 | 38.56 | 13 | 15 | 18 | 26 |
| P60842_IF4A1_HUMAN | EIF4A1 | 46.12 | 9 | 12 | 9 | 13 |
| P33778_H2B1B_HUMAN | HIST1H2BB | 13.94 | 7 | 7 | 9 | 13 |
| P25705_ATPA_HUMAN | ATP5A1 | 59.71 | 13 | 19 | 13 | 19 |
| P60709_ACTB_HUMAN | ACTB | 41.71 | 9 | 10 | 16 | 24 |
| P22087_FBRL_HUMAN | FBL | 33.76 | 7 | 12 | 8 | 12 |
| Q13123_RED_HUMAN | IK | 65.56 | 7 | 11 | 8 | 12 |
| P08579_RU2B_HUMAN | SNRPB2 | 25.47 | 6 | 8 | 8 | 12 |
| O43172_PRP4_HUMAN | PRPF4 | 58.41 | 6 | 9 | 6 | 9 |
| P07437_TBB5_HUMAN | TUBB | 49.64 | 5 | 7 | 6 | 9 |
| P62316_SMD2_HUMAN | SNRPD2 | 13.52 | 4 | 7 | 6 | 9 |
| P32969_RL9_HUMAN | RPL9 | 21.85 | 4 | 5 | 4 | 6 |
| Q96QD9_UIF_HUMAN | FYTTD1 | 35.8 | 4 | 6 | 4 | 6 |
| O95777_LSM8_HUMAN | LSM8 | 10.4 | 2 | 3 | 2 | 3 |
| P33992_MCM5_HUMAN | MCM5 | 82.23 | 2 | 3 | 2 | 3 |
| P35232_PHB_HUMAN | PHB | 29.79 | 2 | 3 | 2 | 3 |
| P39019_RS19_HUMAN | RPS19 | 16.05 | 2 | 3 | 2 | 3 |
| P55060_XPO2_HUMAN | CSE1L | 110.35 | 2 | 3 | 2 | 3 |
| P57721_PCBP3_HUMAN | PCBP3 | 39.44 | 2 | 3 | 2 | 3 |
| P60900_PSA6_HUMAN | PSMA6 | 27.38 | 2 | 3 | 2 | 3 |
| P62847_RS24_HUMAN | RPS24 | 15.41 | 2 | 2 | 2 | 3 |
| P62899_RL31_HUMAN | RPL31 | 14.45 | 2 | 3 | 2 | 3 |
| Q01081_U2AF1_HUMAN | U2AF1 | 27.85 | 2 | 2 | 2 | 3 |
| Q13151_ROA0_HUMAN | HNRNPA0 | 30.82 | 2 | 3 | 2 | 3 |
| Q13185_CBX3_HUMAN | CBX3 | 20.8 | 2 | 3 | 2 | 3 |
| Q14568_HS902_HUMAN | HSP90AA2 | 39.34 | 2 | 2 | 2 | 3 |
| Q71UI9_H2AV_HUMAN | H2AFV | 13.5 | 2 | 3 | 2 | 3 |
| Q86U42_PABP2_HUMAN | PABPN1 | 32.73 | 2 | 3 | 2 | 3 |
| Q99832_TCPH_HUMAN | CCT7 | 59.33 | 2 | 3 | 2 | 3 |
| O00567_NOP56_HUMAN | NOP56 | 66.01 | 13 | 20 | 13 | 20 |
| Q92841_DDX17_HUMAN | DDX17 | 80.22 | 14 | 20 | 14 | 22 |
| Q16352_AINX_HUMAN | INA | 55.36 | 7 | 11 | 7 | 11 |
| P11021_GRP78_HUMAN | HSPA5 | 72.29 | 16 | 25 | 17 | 27 |
| P11142_HSP7C_HUMAN | HSPA8 | 70.85 | 23 | 31 | 25 | 40 |
| P07910_HNRPC_HUMAN | HNRNPC | 33.65 | 7 | 11 | 10 | 16 |
| P17987_TCPA_HUMAN | TCP1 | 60.31 | 5 | 8 | 5 | 8 |
| P31942_HNRH3_HUMAN | HNRNPH3 | 36.9 | 5 | 7 | 5 | 8 |
| P38919_IF4A3_HUMAN | EIF4A3 | 46.84 | 13 | 20 | 13 | 21 |
| P62736_ACTA_HUMAN | ACTA2 | 41.98 | 16 | 23 | 26 | 43 |
| P34931_HS71L_HUMAN | HSPA1L | 70.33 | 6 | 10 | 6 | 10 |
| Q92620_PRP16_HUMAN | DHX38 | 140.42 | 6 | 10 | 6 | 10 |
| O75367_H2AY_HUMAN | H2AFY | 39.59 | 3 | 5 | 3 | 5 |
| O75489_NDUS3_HUMAN | NDUFS3 | 30.22 | 3 | 5 | 3 | 5 |
| P62249_RS16_HUMAN | RPS16 | 16.44 | 3 | 4 | 3 | 5 |
| P62277_RS13_HUMAN | RPS13 | 17.21 | 3 | 5 | 3 | 5 |
| P62314_SMD1_HUMAN | SNRPD1 | 13.27 | 3 | 5 | 3 | 5 |
| Q9UNX3_RL26L_HUMAN | RPL26L1 | 17.25 | 3 | 5 | 3 | 5 |
| Q9UQ35_SRRM2_HUMAN | SRRM2 | 299.44 | 3 | 5 | 3 | 5 |
| Q71U36_TBA1A_HUMAN | TUBA1A | 50.1 | 18 | 27 | 27 | 46 |
| O94906_PRP6_HUMAN | PRPF6 | 106.86 | 7 | 12 | 7 | 12 |
| Q2TAY7_SMU1_HUMAN | SMU1 | 57.51 | 7 | 11 | 7 | 12 |
| Q12874_SF3A3_HUMAN | SF3A3 | 58.81 | 8 | 14 | 8 | 14 |
| O43395_PRPF3_HUMAN | PRPF3 | 77.48 | 4 | 7 | 4 | 7 |
| P78371_TCPB_HUMAN | CCT2 | 57.45 | 4 | 7 | 4 | 7 |
| Q13573_SNW1_HUMAN | SNW1 | 61.46 | 9 | 16 | 9 | 16 |
| P26368_U2AF2_HUMAN | U2AF2 | 53.47 | 10 | 14 | 10 | 18 |
| P08107_HSP71_HUMAN | HSPA1A | 70.01 | 5 | 9 | 5 | 9 |
| P12814_ACTN1_HUMAN | ACTN1 | 102.99 | 5 | 8 | 5 | 9 |
| Q15428_SF3A2_HUMAN | SF3A2 | 49.22 | 4 | 7 | 5 | 9 |
| P38159_RBMX_HUMAN | RBMX | 42.31 | 6 | 11 | 7 | 13 |
| P52272_HNRPM_HUMAN | HNRNPM | 77.46 | 30 | 50 | 31 | 58 |
| P21333_FLNA_HUMAN | FLNA | 280.56 | 8 | 14 | 8 | 15 |
| O43390_HNRPR_HUMAN | HNRNPR | 70.9 | 10 | 18 | 10 | 19 |
| P06396_GELS_HUMAN | GSN | 85.64 | 9 | 19 | 10 | 19 |
| Q13885_TBB2A_HUMAN | TUBB2A | 49.87 | 16 | 26 | 17 | 34 |
| P18583_SON_HUMAN | SON | 263.66 | 7 | 13 | 7 | 14 |
| P55795_HNRH2_HUMAN | HNRNPH2 | 49.23 | 5 | 8 | 5 | 10 |
| O43707_ACTN4_HUMAN | ACTN4 | 104.79 | 4 | 10 | 5 | 10 |
| P33991_MCM4_HUMAN | MCM4 | 96.5 | 4 | 7 | 4 | 8 |
| P62263_RS14_HUMAN | RPS14 | 16.26 | 4 | 8 | 4 | 8 |
| Q96DI7_SNR40_HUMAN | SNRNP40 | 39.29 | 3 | 8 | 4 | 8 |
| P16403_H12_HUMAN | HIST1H1C | 21.35 | 3 | 4 | 3 | 6 |
| P26196_DDX6_HUMAN | DDX6 | 54.38 | 3 | 6 | 3 | 6 |
| P30443_1A01_HUMAN | HLA-A | 40.82 | 3 | 5 | 3 | 6 |
| Q15637_SF01_HUMAN | SF1 | 68.29 | 2 | 6 | 3 | 6 |
| O00303_EIF3F_HUMAN | EIF3F | 37.54 | 2 | 4 | 2 | 4 |
| O75940_SPF30_HUMAN | SMNDC1 | 26.69 | 2 | 4 | 2 | 4 |
| P04843_RPN1_HUMAN | RPN1 | 68.53 | 2 | 4 | 2 | 4 |
| P12235_ADT1_HUMAN | SLC25A4 | 33.04 | 2 | 4 | 2 | 4 |
| P26641_EF1G_HUMAN | EEF1G | 50.09 | 2 | 4 | 2 | 4 |
| P41227_NAA10_HUMAN | NAA10 | 26.44 | 2 | 4 | 2 | 4 |
| P60866_RS20_HUMAN | RPS20 | 13.36 | 2 | 2 | 2 | 4 |
| P62851_RS25_HUMAN | RPS25 | 13.73 | 2 | 4 | 2 | 4 |
| P83731_RL24_HUMAN | RPL24 | 17.77 | 2 | 4 | 2 | 4 |
| Q08945_SSRP1_HUMAN | SSRP1 | 81.02 | 2 | 4 | 2 | 4 |
| Q9NW64_RBM22_HUMAN | RBM22 | 46.87 | 2 | 4 | 2 | 4 |
| P07814_SYEP_HUMAN | EPRS | 170.48 | 1 | 2 | 1 | 2 |
| P10515_ODP2_HUMAN | DLAT | 68.95 | 1 | 2 | 1 | 2 |
| P24534_EF1B_HUMAN | EEF1B2 | 24.75 | 1 | 2 | 1 | 2 |
| P31941_ABC3A_HUMAN | APOBEC3A | 23 | 1 | 2 | 1 | 2 |
| P35606_COPB2_HUMAN | COPB2 | 102.42 | 1 | 1 | 1 | 2 |
| P47755_CAZA2_HUMAN | CAPZA2 | 32.93 | 1 | 2 | 1 | 2 |
| P52565_GDIR1_HUMAN | ARHGDIA | 23.19 | 1 | 2 | 1 | 2 |
| P61026_RAB10_HUMAN | RAB10 | 22.53 | 1 | 2 | 1 | 2 |
| P62873_GBB1_HUMAN | GNB1 | 37.35 | 1 | 2 | 1 | 2 |
| P68366_TBA4A_HUMAN | TUBA4A | 49.89 | 1 | 2 | 1 | 2 |
| Q13162_PRDX4_HUMAN | PRDX4 | 30.52 | 1 | 2 | 1 | 2 |
| Q14576_ELAV3_HUMAN | ELAVL3 | 39.52 | 1 | 2 | 1 | 2 |
| Q16576_RBBP7_HUMAN | RBBP7 | 47.79 | 1 | 2 | 1 | 2 |
| Q2VIR3_IF2GL_HUMAN | EIF2S3L | 51.2 | 1 | 2 | 1 | 2 |
| Q659C4_LAR1B_HUMAN | LARP1B | 105.26 | 1 | 2 | 1 | 2 |
| Q96EP5_DAZP1_HUMAN | DAZAP1 | 43.36 | 1 | 2 | 1 | 2 |
| Q9ULR0_ISY1_HUMAN | ISY1 | 32.97 | 1 | 2 | 1 | 2 |
| Q12906_ILF3_HUMAN | ILF3 | 95.28 | 10 | 20 | 10 | 21 |
| Q12926_ELAV2_HUMAN | ELAVL2 | 39.48 | 8 | 16 | 8 | 17 |
| P08670_VIME_HUMAN | VIM | 53.62 | 7 | 15 | 7 | 15 |
| P52597_HNRPF_HUMAN | HNRNPF | 45.64 | 6 | 10 | 6 | 13 |
| P61247_RS3A_HUMAN | RPS3A | 29.93 | 6 | 13 | 6 | 13 |
| P49327_FAS_HUMAN | FASN | 273.25 | 5 | 11 | 5 | 11 |
| Q9BUJ2_HNRL1_HUMAN | HNRNPUL1 | 95.68 | 9 | 18 | 9 | 20 |
| P40939_ECHA_HUMAN | HADHA | 82.95 | 3 | 6 | 3 | 7 |
| P52907_CAZA1_HUMAN | CAPZA1 | 32.9 | 3 | 7 | 3 | 7 |
| Q1KMD3_HNRL2_HUMAN | HNRNPUL2 | 85.05 | 3 | 7 | 3 | 7 |
| Q02878_RL6_HUMAN | RPL6 | 32.71 | 2 | 7 | 3 | 7 |
| O60506_HNRPQ_HUMAN | SYNCRIP | 69.56 | 8 | 18 | 8 | 19 |
| P18077_RL35A_HUMAN | RPL35A | 12.53 | 2 | 5 | 2 | 5 |
| P18621_RL17_HUMAN | RPL17 | 21.38 | 2 | 5 | 2 | 5 |
| P22695_QCR2_HUMAN | UQCRC2 | 48.41 | 2 | 5 | 2 | 5 |
| P53396_ACLY_HUMAN | ACLY | 120.76 | 2 | 5 | 2 | 5 |
| P55081_MFAP1_HUMAN | MFAP1 | 51.93 | 2 | 5 | 2 | 5 |
| P61353_RL27_HUMAN | RPL27 | 15.79 | 2 | 5 | 2 | 5 |
| P62829_RL23_HUMAN | RPL23 | 14.86 | 2 | 5 | 2 | 5 |
| Q99714_HCD2_HUMAN | HSD17B10 | 26.91 | 2 | 5 | 2 | 5 |
| Q9BQA1_MEP50_HUMAN | WDR77 | 36.7 | 2 | 5 | 2 | 5 |
| Q9Y3Y2_CHTOP_HUMAN | CHTOP | 26.38 | 2 | 5 | 2 | 5 |
| Q86U06_RBM23_HUMAN | RBM23 | 48.7 | 5 | 5 | 14 | 37 |
| P55884_EIF3B_HUMAN | EIF3B | 92.42 | 3 | 6 | 3 | 8 |
| Q09161_NCBP1_HUMAN | NCBP1 | 91.78 | 3 | 8 | 3 | 8 |
| Q5BKZ1_ZN326_HUMAN | ZNF326 | 65.61 | 3 | 8 | 3 | 8 |
| Q96I24_FUBP3_HUMAN | FUBP3 | 61.6 | 3 | 8 | 3 | 8 |
| O14929_HAT1_HUMAN | HAT1 | 49.48 | 2 | 5 | 2 | 6 |
| P05388_RLA0_HUMAN | RPLP0 | 34.25 | 2 | 6 | 2 | 6 |
| P46781_RS9_HUMAN | RPS9 | 22.58 | 2 | 6 | 2 | 6 |
| Q8NHW5_RLA0L_HUMAN | RPLP0P6 | 34.34 | 2 | 6 | 2 | 6 |
| Q9BYX7_ACTBM_HUMAN | POTEKP | 41.99 | 2 | 3 | 2 | 6 |
| P11177_ODPB_HUMAN | PDHB | 39.21 | 1 | 3 | 1 | 3 |
| P27635_RL10_HUMAN | RPL10 | 24.59 | 1 | 3 | 1 | 3 |
| P30876_RPB2_HUMAN | POLR2B | 133.81 | 1 | 2 | 1 | 3 |
| P31689_DNJA1_HUMAN | DNAJA1 | 44.84 | 1 | 3 | 1 | 3 |
| P42167_LAP2B_HUMAN | TMPO | 50.64 | 1 | 3 | 1 | 3 |
| P50914_RL14_HUMAN | RPL14 | 23.42 | 1 | 3 | 1 | 3 |
| P50990_TCPQ_HUMAN | CCT8 | 59.58 | 1 | 3 | 1 | 3 |
| P52292_IMA1_HUMAN | KPNA2 | 57.83 | 1 | 3 | 1 | 3 |
| P61163_ACTZ_HUMAN | ACTR1A | 42.59 | 1 | 3 | 1 | 3 |
| P62857_RS28_HUMAN | RPS28 | 7.84 | 1 | 3 | 1 | 3 |
| Q07020_RL18_HUMAN | RPL18 | 21.62 | 1 | 3 | 1 | 3 |
| Q53F19_CQ085_HUMAN | C17orf85 | 70.55 | 1 | 3 | 1 | 3 |
| Q96KR1_ZFR_HUMAN | ZFR | 116.94 | 1 | 3 | 1 | 3 |
| Q9BQ04_RBM4B_HUMAN | RBM4B | 40.12 | 1 | 3 | 1 | 3 |
| Q9P2K5_MYEF2_HUMAN | MYEF2 | 64.08 | 1 | 3 | 1 | 3 |
| Q9UN86_G3BP2_HUMAN | G3BP2 | 54.09 | 1 | 2 | 1 | 3 |
| Q9UNP9_PPIE_HUMAN | PPIE | 33.41 | 1 | 3 | 1 | 3 |
| Q9Y3F4_STRAP_HUMAN | STRAP | 38.41 | 1 | 3 | 1 | 3 |
| Q9Y5S9_RBM8A_HUMAN | RBM8A | 19.88 | 1 | 3 | 1 | 3 |
| O60306_AQR_HUMAN | AQR | 171.19 | 4 | 13 | 4 | 13 |
| Q92945_FUBP2_HUMAN | KHSRP | 73.07 | 9 | 25 | 9 | 30 |
| P17844_DDX5_HUMAN | DDX5 | 69.1 | 6 | 17 | 6 | 20 |
| P18124_RL7_HUMAN | RPL7 | 29.21 | 3 | 10 | 3 | 10 |
| Q9HCS7_SYF1_HUMAN | XAB2 | 99.95 | 3 | 9 | 3 | 10 |
| P67809_YBOX1_HUMAN | YBX1 | 35.9 | 5 | 10 | 5 | 17 |
| P54886_P5CS_HUMAN | ALDH18A1 | 87.25 | 2 | 7 | 2 | 7 |
| Q99613_EIF3C_HUMAN | EIF3C | 105.28 | 2 | 7 | 2 | 7 |
| Q9BQ39_DDX50_HUMAN | DDX50 | 82.51 | 2 | 7 | 2 | 7 |
| Q9NR30_DDX21_HUMAN | DDX21 | 87.29 | 6 | 21 | 6 | 22 |
| P39023_RL3_HUMAN | RPL3 | 46.08 | 4 | 15 | 4 | 15 |
| O60508_PRP17_HUMAN | CDC40 | 65.48 | 2 | 8 | 2 | 8 |
| P15880_RS2_HUMAN | RPS2 | 31.3 | 2 | 7 | 2 | 8 |
| P62241_RS8_HUMAN | RPS8 | 24.19 | 2 | 7 | 2 | 8 |
| Q15717_ELAV1_HUMAN | ELAVL1 | 36.07 | 2 | 8 | 2 | 8 |
| Q8NAV1_PR38A_HUMAN | PRPF38A | 37.45 | 2 | 6 | 2 | 8 |
| P46776_RL27A_HUMAN | RPL27A | 16.55 | 1 | 4 | 1 | 4 |
| P63173_RL38_HUMAN | RPL38 | 8.21 | 1 | 3 | 1 | 4 |
| P84098_RL19_HUMAN | RPL19 | 23.45 | 1 | 4 | 1 | 4 |
| Q02543_RL18A_HUMAN | RPL18A | 20.75 | 1 | 4 | 1 | 4 |
| Q92598_HS105_HUMAN | HSPH1 | 96.8 | 1 | 4 | 1 | 4 |
| Q9HCG8_CWC22_HUMAN | CWC22 | 105.4 | 1 | 3 | 1 | 4 |
| P62424_RL7A_HUMAN | RPL7A | 29.98 | 2 | 9 | 2 | 10 |
| P46778_RL21_HUMAN | RPL21 | 18.55 | 1 | 5 | 1 | 5 |
| P62753_RS6_HUMAN | RPS6 | 28.66 | 2 | 10 | 2 | 11 |
| Q9Y50L_TNPO3_HUMAN | TNPO3 | 104.14 | 2 | 10 | 2 | 11 |
| P26373_RL13_HUMAN | RPL13 | 24.25 | 1 | 6 | 1 | 6 |
| Q14980_NUMA1_HUMAN | NUMA1 | 238.12 | 1 | 6 | 1 | 6 |
| Q9H0D6_XRN2_HUMAN | XRN2 | 108.51 | 1 | 6 | 1 | 6 |
| P11940_PABP1_HUMAN | PABPC1 | 70.63 | 5 | 29 | 5 | 31 |
| P62750_RL23A_HUMAN | RPL23A | 17.68 | 1 | 7 | 1 | 7 |
| Q02539_H11_HUMAN | HIST1H1A | 21.83 | 1 | 4 | 1 | 11 |
| Q9BZJ0_CRNL1_HUMAN | CRNKL1 | 100.39 | 1 | 13 | 1 | 13 |
| O00425_IF2B3_HUMAN | IGF2BP3 | 63.67 | 1 | 13 | 1 | 14 |
| Q9NZI8_IF2B1_HUMAN | IGF2BP1 | 63.44 | 1 | 19 | 1 | 19 |
| Q7L2E3_DHX30_HUMAN | DHX30 | 133.85 | 0 | 22 | 0 | 23 |
| Q9HCE1_MOV10_HUMAN | MOV10 | 113.6 | 0 | 21 | 0 | 22 |
| Q92900_RENT1_HUMAN | UPF1 | 124.27 | 0 | 19 | 0 | 20 |
| Q6PKG0_LARP1_HUMAN | LARP1 | 123.43 | 0 | 14 | 0 | 16 |
| Q06787_FMR1_HUMAN | FMR1 | 71.13 | 0 | 15 | 0 | 15 |
| P36578_RL4_HUMAN | RPL4 | 47.67 | 0 | 14 | 0 | 14 |
| P51114_FXR1_HUMAN | FXR1 | 69.68 | 0 | 13 | 0 | 13 |
| P51116_FXR2_HUMAN | FXR2 | 74.18 | 0 | 11 | 0 | 11 |
| Q13310_PABP4_HUMAN | PABPC4 | 70.74 | 0 | 9 | 0 | 10 |
| Q16643_DREB_HUMAN | DBN1 | 71.39 | 0 | 9 | 0 | 9 |
| Q9Y6M1_IF2B2_HUMAN | IGF2BP2 | 66.08 | 0 | 8 | 0 | 9 |
| O95831_AIFM1_HUMAN | AIFM1 | 66.86 | 0 | 7 | 0 | 7 |
| P62906_RL10A_HUMAN | RPL10A | 24.82 | 0 | 7 | 0 | 7 |
| Q9BQG0_MBB1A_HUMAN | MYBBP1A | 148.76 | 0 | 7 | 0 | 7 |
| Q9UNF1_MAGD2_HUMAN | MAGED2 | 64.91 | 0 | 7 | 0 | 7 |
| P61313_RL15_HUMAN | RPL15 | 24.13 | 0 | 5 | 0 | 6 |
| Q5JTH9_RRP12_HUMAN | RRP12 | 143.61 | 0 | 5 | 0 | 6 |
| Q6PJT7_ZC3HE_HUMAN | ZC3H14 | 82.82 | 0 | 6 | 0 | 6 |
| Q99700_ATX2_HUMAN | ATXN2 | 140.2 | 0 | 5 | 0 | 6 |
| Q9NZB2_F120A_HUMAN | FAM120A | 121.81 | 0 | 6 | 0 | 6 |
| O76021_RL1D1_HUMAN | RSL1D1 | 54.94 | 0 | 5 | 0 | 5 |
| P02786_TFR1_HUMAN | TFRC | 84.82 | 0 | 5 | 0 | 5 |
| P27708_PYR1_HUMAN | CAD | 242.83 | 0 | 5 | 0 | 5 |
| P46777_RL5_HUMAN | RPL5 | 34.34 | 0 | 5 | 0 | 5 |
| P62917_RL8_HUMAN | RPL8 | 28.01 | 0 | 5 | 0 | 5 |
| Q9NYL9_TMOD3_HUMAN | TMOD3 | 39.57 | 0 | 4 | 0 | 5 |
| Q9Y2W2_WBP11_HUMAN | WBP11 | 69.95 | 0 | 5 | 0 | 5 |
| G3V0J0_G3V0J0_HUMAN | FMR1 | 66.74 | 0 | 3 | 0 | 4 |
| O15226_NKRF_HUMAN | NKRF | 77.62 | 0 | 4 | 0 | 4 |
| O75569_PRKRA_HUMAN | PRKRA | 34.38 | 0 | 4 | 0 | 4 |
| O75821_EIF3G_HUMAN | EIF3G | 35.59 | 0 | 4 | 0 | 4 |
| P30153_2AAA_HUMAN | PPP2R1A | 65.27 | 0 | 4 | 0 | 4 |
| P40429_RL13A_HUMAN | RPL13A | 23.56 | 0 | 4 | 0 | 4 |
| P62280_RS11_HUMAN | RPS11 | 18.42 | 0 | 3 | 0 | 4 |
| Q15057_ACAP2_HUMAN | ACAP2 | 87.97 | 0 | 4 | 0 | 4 |
| Q8IX01_SUGP2_HUMAN | SUGP2 | 120.13 | 0 | 4 | 0 | 4 |
| Q9UHB6_LIMA1_HUMAN | LIMA1 | 85.17 | 0 | 4 | 0 | 4 |
| P00966_ASSY_HUMAN | ASS1 | 46.5 | 0 | 3 | 0 | 3 |
| P16989_YBOX3_HUMAN | YBX3 | 40.07 | 0 | 3 | 0 | 3 |
| P42694_HELZ_HUMAN | HELZ | 218.83 | 0 | 2 | 0 | 3 |
| P46087_NOP2_HUMAN | NOP2 | 89.25 | 0 | 3 | 0 | 3 |
| P51398_RT29_HUMAN | DAP3 | 45.54 | 0 | 2 | 0 | 3 |
| P62312_LSM6_HUMAN | LSM6 | 9.12 | 0 | 3 | 0 | 3 |
| P98170_XIAP_HUMAN | XIAP | 56.65 | 0 | 3 | 0 | 3 |
| Q14204_DYHC1_HUMAN | DYNC1H1 | 532.07 | 0 | 3 | 0 | 3 |
| Q14562_DHX8_HUMAN | DHX8 | 139.23 | 0 | 3 | 0 | 3 |
| Q6NVV1_R13P3_HUMAN | RPL13AP3 | 12.13 | 0 | 3 | 0 | 3 |
| Q8TDN6_BRX1_HUMAN | BRIX1 | 41.37 | 0 | 2 | 0 | 3 |
| Q96EY7_PTCD3_HUMAN | PTCD3 | 78.5 | 0 | 2 | 0 | 3 |
| Q99962_SH3G2_HUMAN | SH3GL2 | 39.94 | 0 | 1 | 0 | 3 |
| Q9BUF5_TBB6_HUMAN | TUBB6 | 49.82 | 0 | 2 | 0 | 3 |
| Q9BXY0_MAK16_HUMAN | MAK16 | 35.35 | 0 | 2 | 0 | 3 |
| Q9H7B2_RPF2_HUMAN | RPF2 | 35.56 | 0 | 3 | 0 | 3 |
| Q9NUD5_ZCHC3_HUMAN | ZCCHC3 | 43.59 | 0 | 3 | 0 | 3 |
| Q9Y262_EIF3L_HUMAN | EIF3L | 66.68 | 0 | 3 | 0 | 3 |
| Q9Y3C6_PPIL1_HUMAN | PPIL1 | 18.23 | 0 | 3 | 0 | 3 |
| Q9Y3U8_RL36_HUMAN | RPL36 | 12.25 | 0 | 3 | 0 | 3 |
| O43347_MSI1H_HUMAN | MSI1 | 39.1 | 0 | 2 | 0 | 2 |
| O75152_ZC11A_HUMAN | ZC3H11A | 89.08 | 0 | 2 | 0 | 2 |
| O95573_ACSL3_HUMAN | ACSL3 | 80.37 | 0 | 2 | 0 | 2 |
| O95793_STAU1_HUMAN | STAU1 | 63.14 | 0 | 2 | 0 | 2 |
| P05023_AT1A1_HUMAN | ATP1A1 | 112.82 | 0 | 2 | 0 | 2 |
| P20671_H2A1D_HUMAN | HIST1H2AD | 14.1 | 0 | 2 | 0 | 2 |
| P26639_SYTC_HUMAN | TARS | 83.38 | 0 | 2 | 0 | 2 |
| P42766_RL35_HUMAN | RPL35 | 14.54 | 0 | 2 | 0 | 2 |
| P46013_KI67_HUMAN | MKI67 | 358.47 | 0 | 2 | 0 | 2 |
| P46779_RL28_HUMAN | RPL28 | 15.74 | 0 | 2 | 0 | 2 |
| P48634_PRC2A_HUMAN | PRRC2A | 228.72 | 0 | 2 | 0 | 2 |
| P55084_ECHB_HUMAN | HADHB | 51.26 | 0 | 2 | 0 | 2 |
| P56192_SYMC_HUMAN | MARS | 101.05 | 0 | 2 | 0 | 2 |
| P61158_ARP3_HUMAN | ACTR3 | 47.34 | 0 | 2 | 0 | 2 |
| P62306_RUXF_HUMAN | SNRPF | 9.72 | 0 | 2 | 0 | 2 |
| P62834_RAP1A_HUMAN | RAP1A | 20.97 | 0 | 2 | 0 | 2 |
| P62888_RL30_HUMAN | RPL30 | 12.78 | 0 | 2 | 0 | 2 |
| P83881_RL36A_HUMAN | RPL36A | 12.43 | 0 | 2 | 0 | 2 |
| Q00577_PURA_HUMAN | PURA | 34.89 | 0 | 2 | 0 | 2 |
| Q07666_KHDR1_HUMAN | KHDRBS1 | 48.2 | 0 | 2 | 0 | 2 |
| Q13347_EIF3I_HUMAN | EIF3I | 36.48 | 0 | 2 | 0 | 2 |
| Q14157_UBP2L_HUMAN | UBAP2L | 114.47 | 0 | 2 | 0 | 2 |
| Q15424_SAFB1_HUMAN | SAFB | 102.58 | 0 | 2 | 0 | 2 |
| Q15758_AAAT_HUMAN | SLC1A5 | 56.56 | 0 | 2 | 0 | 2 |
| Q3MHD2_LSM12_HUMAN | LSM12 | 21.69 | 0 | 1 | 0 | 2 |
| Q58FF3_ENPLL_HUMAN | HSP90B2P | 45.83 | 0 | 2 | 0 | 2 |
| Q6NYC1_JMJD6_HUMAN | JMJD6 | 46.43 | 0 | 2 | 0 | 2 |
| Q71RC2_LARP4_HUMAN | LARP4 | 80.55 | 0 | 2 | 0 | 2 |
| Q7Z417_NUFP2_HUMAN | NUFIP2 | 76.07 | 0 | 1 | 0 | 2 |
| Q8IX18_DHX40_HUMAN | DHX40 | 88.5 | 0 | 2 | 0 | 2 |
| Q8NF91_SYNE1_HUMAN | SYNE1 | 1010.46 | 0 | 1 | 0 | 2 |
| Q8TB72_PUM2_HUMAN | PUM2 | 114.14 | 0 | 2 | 0 | 2 |
| Q8WXX5_DNJC9_HUMAN | DNAJC9 | 29.89 | 0 | 1 | 0 | 2 |
| Q92879_CELF1_HUMAN | CELF1 | 52.03 | 0 | 2 | 0 | 2 |
| Q96DH6_MSI2H_HUMAN | MSI2 | 35.17 | 0 | 1 | 0 | 2 |
| Q96L21_RL10L_HUMAN | RPL10L | 24.5 | 0 | 2 | 0 | 2 |
| Q99873_ANM1_HUMAN | PRMT1 | 41.49 | 0 | 2 | 0 | 2 |
| Q9BV38_WDR18_HUMAN | WDR18 | 47.38 | 0 | 2 | 0 | 2 |
| Q9BVJ6_UT14A_HUMAN | UTP14A | 87.92 | 0 | 2 | 0 | 2 |
| Q9BYJ9_YTHD1_HUMAN | YTHDF1 | 60.84 | 0 | 2 | 0 | 2 |
| Q9GZR7_DDX24_HUMAN | DDX24 | 96.27 | 0 | 2 | 0 | 2 |
| Q9H0G5_NSRP1_HUMAN | NSRP1 | 66.35 | 0 | 2 | 0 | 2 |
| Q9H3K6_BOLA2_HUMAN | BOLA2 | 10.11 | 0 | 2 | 0 | 2 |
| Q9H583_HEAT1_HUMAN | HEATR1 | 242.22 | 0 | 2 | 0 | 2 |
| Q9H6R4_NOL6_HUMAN | NOL6 | 127.51 | 0 | 2 | 0 | 2 |
| Q9NS69_TOM22_HUMAN | TOMM22 | 15.51 | 0 | 2 | 0 | 2 |
| Q9NUL3_STAU2_HUMAN | STAU2 | 62.6 | 0 | 2 | 0 | 2 |
| Q9NZR1_TMOD2_HUMAN | TMOD2 | 39.57 | 0 | 2 | 0 | 2 |
| Q9UBS4_DJB11_HUMAN | DNAJB11 | 40.49 | 0 | 2 | 0 | 2 |
| Q9UDY2_ZO2_HUMAN | TJP2 | 133.88 | 0 | 2 | 0 | 2 |
| Q9UH17_ABC3B_HUMAN | APOBEC3B | 45.89 | 0 | 2 | 0 | 2 |
| Q9Y3B7_RM11_HUMAN | MRPL11 | 20.67 | 0 | 2 | 0 | 2 |
| Q9Y520_PRC2C_HUMAN | PRRC2C | 316.72 | 0 | 2 | 0 | 2 |
| Q9Y5A9_YTHD2_HUMAN | YTHDF2 | 62.3 | 0 | 2 | 0 | 2 |
| Q9Y676_RT18B_HUMAN | MRPS18B | 29.38 | 0 | 2 | 0 | 2 |
| A0A087WYL5_A0A087WYL5_HUMAN | SEZ6L2 | 99.07 | 0 | 1 | 0 | 1 |
| A8MX80_YM017_HUMAN | | 37.61 | 0 | 1 | 0 | 1 |
| B7Z596_B7Z596_HUMAN | TPM1 | 31.73 | 0 | 1 | 0 | 1 |
| E9PK91_E9PK91_HUMAN | BCLAF1 | 100.35 | 0 | 1 | 0 | 1 |
| F8VRH0_F8VRH0_HUMAN | PCBP2 | 32.02 | 0 | 1 | 0 | 1 |
| F8VU44_F8VU44_HUMAN | NUDT14 | 10.61 | 0 | 1 | 0 | 1 |
| F8WDC4_F8WDC4_HUMAN | POGLUT1 | 12.38 | 0 | 1 | 0 | 1 |
| G3V542_G3V542_HUMAN | TUBB3 | 4.97 | 0 | 1 | 0 | 1 |
| H7C1M2_H7C1M2_HUMAN | SON | 159.92 | 0 | 1 | 0 | 1 |
| O14617_AP3D1_HUMAN | AP3D1 | 130.08 | 0 | 1 | 0 | 1 |
| O15234_CASC3_HUMAN | CASC3 | 76.23 | 0 | 1 | 0 | 1 |
| O15372_EIF3H_HUMAN | EIF3H | 39.91 | 0 | 1 | 0 | 1 |
| O15397_IPO8_HUMAN | IPO8 | 119.86 | 0 | 1 | 0 | 1 |
| O15455_TLR3_HUMAN | TLR3 | 103.76 | 0 | 1 | 0 | 1 |
| O43251_RFOX2_HUMAN | RBFOX2 | 41.35 | 0 | 1 | 0 | 1 |
| O43660_PLRG1_HUMAN | PLRG1 | 57.16 | 0 | 1 | 0 | 1 |
| O43776_SYNC_HUMAN | NARS | 62.9 | 0 | 1 | 0 | 1 |
| O43823_AKAP8_HUMAN | AKAP8 | 76.06 | 0 | 1 | 0 | 1 |
| O60341_KDM1A_HUMAN | KDM1A | 92.84 | 0 | 1 | 0 | 1 |
| O60783_RT14_HUMAN | MRPS14 | 15.13 | 0 | 1 | 0 | 1 |
| O60828_PQBP1_HUMAN | PQBP1 | 30.45 | 0 | 1 | 0 | 1 |
| O75083_WDR1_HUMAN | WDR1 | 66.15 | 0 | 1 | 0 | 1 |
| O75475_PSIP1_HUMAN | PSIP1 | 60.07 | 0 | 1 | 0 | 1 |
| O75531_BAF_HUMAN | BANF1 | 10.05 | 0 | 1 | 0 | 1 |
| O75554_WBP4_HUMAN | WBP4 | 42.48 | 0 | 1 | 0 | 1 |
| P05387_RLA2_HUMAN | RPLP2 | 11.66 | 0 | 1 | 0 | 1 |
| P09234_RU1C_HUMAN | SNRPC | 17.38 | 0 | 1 | 0 | 1 |
| P09960_LKHA4_HUMAN | LTA4H | 69.24 | 0 | 1 | 0 | 1 |
| P10412_H14_HUMAN | HIST1H1E | 21.85 | 0 | 1 | 0 | 1 |
| P12236_ADT3_HUMAN | SLC25A6 | 32.85 | 0 | 1 | 0 | 1 |
| P12532_KCRU_HUMAN | CKMT1A | 47.01 | 0 | 1 | 0 | 1 |
| P13073_COX41_HUMAN | COX4I1 | 19.56 | 0 | 1 | 0 | 1 |
| P17812_PYRG1_HUMAN | CTPS1 | 66.65 | 0 | 1 | 0 | 1 |
| P18754_RCC1_HUMAN | RCC1 | 44.94 | 0 | 1 | 0 | 1 |
| P26640_SYVC_HUMAN | VARS | 140.39 | 0 | 1 | 0 | 1 |
| P31930_QCR1_HUMAN | UQCRC1 | 52.61 | 0 | 1 | 0 | 1 |
| P35749_MYH11_HUMAN | MYH11 | 227.2 | 0 | 1 | 0 | 1 |
| P38432_COIL_HUMAN | COIL | 62.57 | 0 | 1 | 0 | 1 |
| P42768_WASP_HUMAN | WAS | 52.88 | 0 | 1 | 0 | 1 |
| P43355_MAGA1_HUMAN | MAGEA1 | 34.32 | 0 | 1 | 0 | 1 |
| P46459_NSF_HUMAN | NSF | 82.54 | 0 | 1 | 0 | 1 |
| P47914_RL29_HUMAN | RPL29 | 17.74 | 0 | 1 | 0 | 1 |
| P49761_CLK3_HUMAN | CLK3 | 73.47 | 0 | 1 | 0 | 1 |
| P50213_IDH3A_HUMAN | IDH3A | 39.57 | 0 | 1 | 0 | 1 |
| P51659_DHB4_HUMAN | HSD17B4 | 79.64 | 0 | 1 | 0 | 1 |
| P52209_6PGD_HUMAN | PGD | 53.11 | 0 | 1 | 0 | 1 |
| P53618_COPB_HUMAN | COPB1 | 107.07 | 0 | 1 | 0 | 1 |
| P55209_NP1L1_HUMAN | NAP1L1 | 45.35 | 0 | 1 | 0 | 1 |
| P55769_NH2L1_HUMAN | NHP2L1 | 14.16 | 0 | 1 | 0 | 1 |
| P60953_CDC42_HUMAN | CDC42 | 21.25 | 0 | 1 | 0 | 1 |
| P61019_RAB2A_HUMAN | RAB2A | 23.53 | 0 | 1 | 0 | 1 |
| P61513_RL37A_HUMAN | RPL37A | 10.27 | 0 | 1 | 0 | 1 |
| P61764_STXB1_HUMAN | STXBP1 | 67.53 | 0 | 1 | 0 | 1 |
| P62861_RS30_HUMAN | FAU | 6.64 | 0 | 1 | 0 | 1 |
| P62910_RL32_HUMAN | RPL32 | 15.85 | 0 | 1 | 0 | 1 |
| P63151_2ABA_HUMAN | PPP2R2A | 51.66 | 0 | 1 | 0 | 1 |
| P63167_DYL1_HUMAN | DYNLL1 | 10.36 | 0 | 1 | 0 | 1 |
| P78362_SRPK2_HUMAN | SRPK2 | 77.48 | 0 | 1 | 0 | 1 |
| P82650_RT22_HUMAN | MRPS22 | 41.25 | 0 | 1 | 0 | 1 |
| P82673_RT35_HUMAN | MRPS35 | 36.82 | 0 | 1 | 0 | 1 |
| Q00059_TFAM_HUMAN | TFAM | 29.08 | 0 | 1 | 0 | 1 |
| Q01130_SRSF2_HUMAN | SRSF2 | 25.46 | 0 | 1 | 0 | 1 |
| Q02252_MMSA_HUMAN | ALDH6A1 | 57.8 | 0 | 1 | 0 | 1 |
| Q04637_IF4G1_HUMAN | EIF4G1 | 175.38 | 0 | 1 | 0 | 1 |
| Q08426_ECHP_HUMAN | EHHADH | 79.44 | 0 | 1 | 0 | 1 |
| Q13257_MD2L1_HUMAN | MAD2L1 | 23.5 | 0 | 1 | 0 | 1 |
| Q13561_DCTN2_HUMAN | DCTN2 | 44.2 | 0 | 1 | 0 | 1 |
| Q13601_KRR1_HUMAN | KRR1 | 43.64 | 0 | 1 | 0 | 1 |
| Q13813_SPTN1_HUMAN | SPTAN1 | 284.36 | 0 | 1 | 0 | 1 |
| Q13882_PTK6_HUMAN | PTK6 | 51.8 | 0 | 1 | 0 | 1 |
| Q14151_SAFB2_HUMAN | SAFB2 | 107.41 | 0 | 1 | 0 | 1 |
| Q14671_PUM1_HUMAN | PUM1 | 126.39 | 0 | 1 | 0 | 1 |
| Q15008_PSMD6_HUMAN | PSMD6 | 45.5 | 0 | 1 | 0 | 1 |
| Q15050_RRS1_HUMAN | RRS1 | 41.17 | 0 | 1 | 0 | 1 |
| Q15397_K0020_HUMAN | KIAA0020 | 73.54 | 0 | 1 | 0 | 1 |
| Q16650_TBR1_HUMAN | TBR1 | 74.01 | 0 | 1 | 0 | 1 |
| Q3KQU3_MA7D1_HUMAN | MAP7D1 | 92.76 | 0 | 1 | 0 | 1 |
| Q5SSJ5_HP1B3_HUMAN | HP1BP3 | 61.17 | 0 | 1 | 0 | 1 |
| Q5VIY5_ZN468_HUMAN | ZNF468 | 60.53 | 0 | 1 | 0 | 1 |
| Q86X67_NUD13_HUMAN | NUDT13 | 39.66 | 0 | 1 | 0 | 1 |
| Q8IWZ3_ANKH1_HUMAN | ANKHD1 | 269.29 | 0 | 1 | 0 | 1 |
| Q8NEE0_KLAS1_HUMAN | KLHL30-AS1 | 9.66 | 0 | 1 | 0 | 1 |
| Q8NEK5_ZN548_HUMAN | ZNF548 | 62.69 | 0 | 1 | 0 | 1 |
| Q8WWM7_ATX2L_HUMAN | ATXN2L | 113.3 | 0 | 1 | 0 | 1 |
| Q8WXG6_MADD_HUMAN | MADD | 183.19 | 0 | 1 | 0 | 1 |
| Q92522_H1X_HUMAN | H1FX | 22.47 | 0 | 1 | 0 | 1 |
| Q92552_RT27_HUMAN | MRPS27 | 47.58 | 0 | 1 | 0 | 1 |
| Q92616_GCN1L_HUMAN | GCN1L1 | 292.57 | 0 | 1 | 0 | 1 |
| Q92901_RL3L_HUMAN | RPL3L | 46.27 | 0 | 1 | 0 | 1 |
| Q96C19_EFHD2_HUMAN | EFHD2 | 26.68 | 0 | 1 | 0 | 1 |
| Q96SB4_SRPK1_HUMAN | SRPK1 | 74.28 | 0 | 1 | 0 | 1 |
| Q96T76_MMS19_HUMAN | MMS19 | 113.22 | 0 | 1 | 0 | 1 |
| Q99729_ROAA_HUMAN | HNRNPAB | 36.2 | 0 | 1 | 0 | 1 |
| Q99848_EBP2_HUMAN | EBNA1BP2 | 34.83 | 0 | 1 | 0 | 1 |
| Q9BVP2_GNL3_HUMAN | GNL3 | 61.95 | 0 | 1 | 0 | 1 |
| Q9BYG3_MK67I_HUMAN | NIFK | 34.2 | 0 | 1 | 0 | 1 |
| Q9H2U1_DHX36_HUMAN | DHX36 | 114.69 | 0 | 1 | 0 | 1 |
| Q9H3N1_TMX1_HUMAN | TMX1 | 31.77 | 0 | 1 | 0 | 1 |
| Q9H4B6_SAV1_HUMAN | SAV1 | 44.61 | 0 | 1 | 0 | 1 |
| Q9H814_PHAX_HUMAN | PHAX | 44.38 | 0 | 1 | 0 | 1 |
| Q9NR28_DBLOH_HUMAN | DIABLO | 27.11 | 0 | 1 | 0 | 1 |
| Q9NR31_SAR1A_HUMAN | SAR1A | 22.35 | 0 | 1 | 0 | 1 |
| Q9NR56_MBNL1_HUMAN | MBNL1 | 41.79 | 0 | 1 | 0 | 1 |
| Q9NRR4_RNC_HUMAN | DROSHA | 159.21 | 0 | 1 | 0 | 1 |
| Q9NUQ9_FA49B_HUMAN | FAM49B | 36.72 | 0 | 1 | 0 | 1 |
| Q9NX05_F120C_HUMAN | FAM120C | 120.51 | 0 | 1 | 0 | 1 |
| Q9NYK5_RM39_HUMAN | MRPL39 | 38.69 | 0 | 1 | 0 | 1 |
| Q9NZL4_HPBP1_HUMAN | HSPBP1 | 39.45 | 0 | 1 | 0 | 1 |
| Q9P035_HACD3_HUMAN | PTPLAD1 | 43.13 | 0 | 1 | 0 | 1 |
| Q9P2I0_CPSF2_HUMAN | CPSF2 | 88.43 | 0 | 1 | 0 | 1 |
| Q9UKJ3_GPTC8_HUMAN | GPATCH8 | 164.1 | 0 | 1 | 0 | 1 |
| Q9UQ03_COR2B_HUMAN | CORO2B | 54.92 | 0 | 1 | 0 | 1 |
| Q9Y266_NUDC_HUMAN | NUDC | 38.22 | 0 | 1 | 0 | 1 |
| Q9Y2L1_RRP44_HUMAN | DIS3 | 108.93 | 0 | 1 | 0 | 1 |
| Q9Y2R4_DDX52_HUMAN | DDX52 | 67.46 | 0 | 1 | 0 | 1 |
| Q9Y4W2_LAS1L_HUMAN | LAS1L | 83.01 | 0 | 1 | 0 | 1 |
| Q9Y580_RBM7_HUMAN | RBM7 | 30.48 | 0 | 1 | 0 | 1 |
| Q9Y5V3_MAGD1_HUMAN | MAGED1 | 86.11 | 0 | 1 | 0 | 1 |

TABLE 2

| | | | | RBM39- |
|---|---|---|---|---|
| reference | Gene Symbol | MWT (kDa) | Control total | 3xFLAG total |
| Q14498_RBM39_HUMAN | RBM39 | 59.34 | 2 | 423 |
| Q15365_PCBP1_HUMAN | PCBP1 | 37.47 | 1 | 133 |
| Q6P2Q9_PRP8_HUMAN | PRPF8 | 273.43 | 3 | 128 |
| O75643_U520_HUMAN | SNRNP200 | 244.35 | 4 | 114 |
| P62736_ACTA_HUMAN | ACTA2 | 41.98 | 375 | 104 |
| O75533_SF3B1_HUMAN | SF3B1 | 145.74 | 3 | 77 |
| P23588_IF4B_HUMAN | EIF4B | 69.11 | 37 | 64 |
| Q13435_SF3B2_HUMAN | SF3B2 | 100.16 | 2 | 55 |
| Q13838_DX39B_HUMAN | DDX39B | 48.96 | 7 | 54 |
| Q86U06_RBM23_HUMAN | RBM23 | 48.7 | 0 | 52 |
| Q15393_SF3B3_HUMAN | SF3B3 | 135.49 | 6 | 50 |
| Q15029_U5S1_HUMAN | EFTUD2 | 109.37 | 3 | 48 |
| P52272_HNRPM_HUMAN | HNRNPM | 77.46 | 2 | 47 |
| P07910_HNRPC_HUMAN | HNRNPC | 33.65 | 3 | 44 |
| Q15459_SF3A1_HUMAN | SF3A1 | 88.83 | 1 | 43 |
| Q15366_PCBP2_HUMAN | PCBP2 | 38.56 | 1 | 40 |
| Q71U36_TBA1A_HUMAN | TUBA1A | 50.1 | 19 | 40 |
| P11142_HSP7C_HUMAN | HSPA8 | 70.85 | 25 | 37 |
| P60709_ACTB_HUMAN | ACTB | 41.71 | 224 | 36 |
| P18583_SON_HUMAN | SON | 263.66 | 0 | 35 |
| P38919_IF4A3_HUMAN | EIF4A3 | 46.84 | 8 | 35 |
| Q9NYF8_BCLF1_HUMAN | BCLAF1 | 106.06 | 65 | 34 |
| O00422_SAP18_HUMAN | SAP18 | 17.55 | 0 | 33 |
| O43143_DHX15_HUMAN | DHX15 | 90.88 | 5 | 32 |
| Q99459_CDC5L_HUMAN | CDC5L | 92.19 | 0 | 30 |
| P98175_RBM10_HUMAN | RBM10 | 103.47 | 38 | 30 |
| P43243_MATR3_HUMAN | MATR3 | 94.56 | 6 | 28 |
| P11021_GRP78_HUMAN | HSPA5 | 72.29 | 10 | 28 |
| P22626_ROA2_HUMAN | HNRNPA2B1 | 37.41 | 7 | 27 |
| O60306_AQR_HUMAN | AQR | 171.19 | 0 | 26 |
| P38646_GRP75_HUMAN | HSPA9 | 73.63 | 5 | 24 |
| P61978_HNRPK_HUMAN | HNRNPK | 50.94 | 13 | 24 |
| P14866_HNRPL_HUMAN | HNRNPL | 64.09 | 3 | 23 |
| Q9Y2W1_TR150_HUMAN | THRAP3 | 108.6 | 51 | 23 |
| Q13573_SNW1_HUMAN | SNW1 | 61.46 | 0 | 22 |
| P11940_PABP1_HUMAN | PABPC1 | 70.63 | 6 | 22 |
| P78332_RBM6_HUMAN | RBM6 | 128.57 | 23 | 22 |
| O60506_HNRPQ_HUMAN | SYNCRIP | 69.56 | 0 | 21 |
| Q6PJT7_ZC3HE_HUMAN | ZC3H14 | 82.82 | 0 | 21 |
| Q9H307_PININ_HUMAN | PNN | 81.56 | 0 | 21 |
| P12956_XRCC6_HUMAN | XRCC6 | 69.8 | 7 | 21 |
| Q13885_TBB2A_HUMAN | TUBB2A | 49.87 | 13 | 21 |
| Q12906_ILF3_HUMAN | ILF3 | 95.28 | 1 | 20 |
| P67809_YBOX1_HUMAN | YBX1 | 35.9 | 4 | 20 |
| O43395_PRPF3_HUMAN | PRPF3 | 77.48 | 0 | 19 |
| Q08211_DHX9_HUMAN | DHX9 | 140.87 | 0 | 19 |
| Q2TAY7_SMU1_HUMAN | SMU1 | 57.51 | 0 | 19 |
| Q9BZJ0_CRNL1_HUMAN | CRNKL1 | 100.39 | 0 | 19 |
| Q9BUJ2_HNRL1_HUMAN | HNRNPUL1 | 95.68 | 1 | 19 |
| P51991_ROA3_HUMAN | HNRNPA3 | 39.57 | 3 | 19 |
| P84090_ERH_HUMAN | ERH | 12.25 | 8 | 19 |
| Q92620_PRP16_HUMAN | DHX38 | 140.42 | 13 | 19 |
| O00567_NOP56_HUMAN | NOP56 | 66.01 | 0 | 18 |
| P78527_PRKDC_HUMAN | PRKDC | 468.79 | 0 | 18 |
| Q7L2E3_DHX30_HUMAN | DHX30 | 133.85 | 0 | 18 |
| Q92900_RENT1_HUMAN | UPF1 | 124.27 | 0 | 18 |
| Q9HCE1_MOV10_HUMAN | MOV10 | 113.6 | 0 | 18 |
| P31943_HNRH1_HUMAN | HNRNPH1 | 49.2 | 8 | 18 |
| O43390_HNRPR_HUMAN | HNRNPR | 70.9 | 0 | 17 |
| O94906_PRP6_HUMAN | PRPF6 | 106.86 | 0 | 17 |
| Q6PKG0_LARP1_HUMAN | LARP1 | 123.43 | 0 | 17 |
| Q92945_FUBP2_HUMAN | KHSRP | 73.07 | 1 | 17 |
| Q12926_ELAV2_HUMAN | ELAVL2 | 39.48 | 5 | 17 |
| Q06787_FMR1_HUMAN | FMR1 | 71.13 | 13 | 17 |
| O43290_SNUT1_HUMAN | SART1 | 90.2 | 0 | 16 |
| Q09161_NCBP1_HUMAN | NCBP1 | 91.78 | 0 | 16 |
| Q96QD9_UIF_HUMAN | FYTTD1 | 35.8 | 0 | 16 |
| Q9HCS7_SYF1_HUMAN | XAB2 | 99.95 | 0 | 16 |
| Q9Y2X3_NOP58_HUMAN | NOP58 | 59.54 | 0 | 16 |
| P31942_HNRH3_HUMAN | HNRNPH3 | 36.9 | 3 | 16 |
| P52597_HNRPF_HUMAN | HNRNPF | 45.64 | 3 | 16 |
| Q9NR30_DDX21_HUMAN | DDX21 | 87.29 | 4 | 16 |
| P23396_RS3_HUMAN | RPS3 | 26.67 | 5 | 16 |
| P09874_PARP1_HUMAN | PARP1 | 113.01 | 40 | 16 |
| Q13123_RED_HUMAN | IK | 65.56 | 0 | 15 |
| P09661_RU2A_HUMAN | SNRPA1 | 28.4 | 1 | 15 |
| Q9NZI8_IF2B1_HUMAN | IGF2BP1 | 63.44 | 1 | 15 |
| P51114_FXR1_HUMAN | FXR1 | 69.68 | 2 | 15 |
| P62316_SMD2_HUMAN | SNRPD2 | 13.52 | 2 | 15 |
| Q12905_ILF2_HUMAN | ILF2 | 43.04 | 2 | 15 |
| P49327_FAS_HUMAN | FASN | 273.25 | 8 | 15 |
| Q15208_STK38_HUMAN | STK38 | 54.16 | 20 | 15 |
| O75152_ZC11A_HUMAN | ZC3H11A | 89.08 | 0 | 14 |
| Q92841_DDX17_HUMAN | DDX17 | 80.22 | 0 | 14 |
| Q9UKM9_RALY_HUMAN | RALY | 32.44 | 0 | 14 |
| Q9Y5L0_TNPO3_HUMAN | TNPO3 | 104.14 | 0 | 14 |
| P13010_XRCC5_HUMAN | XRCC5 | 82.65 | 1 | 14 |
| P14678_RSMB_HUMAN | SNRPB | 24.59 | 2 | 14 |
| Q13509_TBB3_HUMAN | TUBB3 | 50.4 | 5 | 14 |
| P25705_ATPA_HUMAN | ATP5A1 | 59.71 | 6 | 14 |
| G3XAC6_G3XAC6_HUMAN | RBM39 | 47.99 | 0 | 13 |
| O00425_IF2B3_HUMAN | IGF2BP3 | 63.67 | 0 | 13 |
| O60508_PRP17_HUMAN | CDC40 | 65.48 | 0 | 13 |
| P22087_FBRL_HUMAN | FBL | 33.76 | 0 | 13 |
| P55265_DSRAD_HUMAN | ADAR | 135.98 | 0 | 13 |
| Q8IX01_SUGP2_HUMAN | SUGP2 | 120.13 | 0 | 13 |
| Q8WWY3_PRP31_HUMAN | PRPF31 | 55.42 | 0 | 13 |
| Q96DI7_SNR40_HUMAN | SNRNP40 | 39.29 | 0 | 13 |
| Q9UMS4_PRP19_HUMAN | PRPF19 | 55.15 | 0 | 13 |
| O00148_DX39A_HUMAN | DDX39A | 49.1 | 1 | 13 |
| P06576_ATPB_HUMAN | ATP5B | 56.52 | 4 | 13 |
| P13639_EF2_HUMAN | EEF2 | 95.28 | 7 | 13 |
| P08670_VIME_HUMAN | VIM | 53.62 | 63 | 13 |
| O60231_DHX16_HUMAN | DHX16 | 119.19 | 0 | 12 |
| P08579_RU2B_HUMAN | SNRPB2 | 25.47 | 0 | 12 |
| Q07955_SRSF1_HUMAN | SRSF1 | 27.73 | 0 | 12 |
| Q5BKZ1_ZN326_HUMAN | ZNF326 | 65.61 | 0 | 12 |
| Q9Y2W2_WBP11_HUMAN | WBP11 | 69.95 | 0 | 12 |
| B7ZW38_HNRC3_HUMAN | HNRNPCL3 | 32.01 | 3 | 12 |
| P41219_PERI_HUMAN | PRPH | 53.62 | 44 | 12 |
| P26368_U2AF2_HUMAN | U2AF2 | 53.47 | 0 | 11 |
| Q14562_DHX8_HUMAN | DHX8 | 139.23 | 0 | 11 |
| Q86XP3_DDX42_HUMAN | DDX42 | 102.91 | 0 | 11 |
| Q96I24_FUBP3_HUMAN | FUBP3 | 61.6 | 0 | 11 |
| P51116_FXR2_HUMAN | FXR2 | 74.18 | 3 | 11 |
| P34931_HS71L_HUMAN | HSPA1L | 70.33 | 4 | 11 |
| P68104_EF1A1_HUMAN | EEF1A1 | 50.11 | 8 | 11 |
| Q32P51_RA1L2_HUMAN | HNRNPA1L2 | 34.2 | 8 | 11 |
| P62805_H4_HUMAN | HIST1H4A | 11.36 | 9 | 11 |
| P04406_G3P_HUMAN | GAPDH | 36.03 | 10 | 11 |
| O14744_ANM5_HUMAN | PRMT5 | 72.64 | 16 | 11 |
| O43172_PRP4_HUMAN | PRPF4 | 58.41 | 0 | 10 |
| Q53F19_CQ085_HUMAN | C17orf85 | 70.55 | 0 | 10 |
| Q9BY77_PDIP3_HUMAN | POLDIP3 | 46.06 | 0 | 10 |
| P46781_RS9_HUMAN | RPS9 | 22.58 | 1 | 10 |
| Q13263_TIF1B_HUMAN | TRIM28 | 88.49 | 1 | 10 |
| Q15717_ELAV1_HUMAN | ELAVL1 | 36.07 | 1 | 10 |
| Q86U42_PABP2_HUMAN | PABPN1 | 32.73 | 1 | 10 |
| P18124_RL7_HUMAN | RPL7 | 29.21 | 2 | 10 |
| P82979_SARNP_HUMAN | SARNP | 23.66 | 2 | 10 |
| P62829_RL23_HUMAN | RPL23 | 14.86 | 3 | 10 |
| P10809_CH60_HUMAN | HSPD1 | 61.02 | 4 | 10 |
| P60842_IF4A1_HUMAN | EIF4A1 | 46.12 | 5 | 10 |
| Q14152_EIF3A_HUMAN | EIF3A | 166.47 | 6 | 10 |
| O43852_CALU_HUMAN | CALU | 37.08 | 10 | 10 |
| P14618_KPYM_HUMAN | PKM | 57.9 | 10 | 10 |
| Q16352_AINX_HUMAN | INA | 55.36 | 35 | 10 |
| Q16643_DREB_HUMAN | DBN1 | 71.39 | 41 | 10 |
| P42285_SK2L2_HUMAN | SKIV2L2 | 117.73 | 0 | 9 |
| P49736_MCM2_HUMAN | MCM2 | 101.83 | 0 | 9 |
| P62995_TRA2B_HUMAN | TRA2B | 33.65 | 0 | 9 |
| Q12874_SF3A3_HUMAN | SF3A3 | 58.81 | 0 | 9 |
| Q13310_PABP4_HUMAN | PABPC4 | 70.74 | 0 | 9 |
| Q15287_RNPS1_HUMAN | RNPS1 | 34.19 | 0 | 9 |
| Q8N163_CCAR2_HUMAN | CCAR2 | 102.84 | 0 | 9 |
| Q8NAV1_PR38A_HUMAN | PRPF38A | 37.45 | 0 | 9 |
| Q9UHX1_PUF60_HUMAN | PUF60 | 59.84 | 0 | 9 |
| P11388_TOP2A_HUMAN | TOP2A | 174.28 | 1 | 9 |
| Q9Y559_RBM8A_HUMAN | RBM8A | 19.88 | 1 | 9 |
| P25205_MCM3_HUMAN | MCM3 | 90.92 | 2 | 9 |
| P26599_PTBP1_HUMAN | PTBP1 | 57.19 | 2 | 9 |
| P33993_MCM7_HUMAN | MCM7 | 81.26 | 2 | 9 |
| P55795_HNRH2_HUMAN | HNRNPH2 | 49.23 | 3 | 9 |
| Q00839_HNRPU_HUMAN | HNRNPU | 90.53 | 4 | 9 |
| P07437_TBB5_HUMAN | TUBB | 49.64 | 5 | 9 |
| Q8WUA2_PPIL4_HUMAN | PPIL4 | 57.19 | 5 | 9 |
| P06733_ENOA_HUMAN | ENO1 | 47.14 | 13 | 9 |
| P06396_GELS_HUMAN | GSN | 85.64 | 54 | 9 |
| P52907_CAZA1_HUMAN | CAPZA1 | 32.9 | 63 | 9 |
| P17987_TCPA_HUMAN | TCP1 | 60.31 | 0 | 8 |
| P26196_DDX6_HUMAN | DDX6 | 54.38 | 0 | 8 |
| Q15428_SF3A2_HUMAN | SF3A2 | 49.22 | 0 | 8 |
| Q96A72_MGN2_HUMAN | MAGOHB | 17.26 | 0 | 8 |
| Q9UNF1_MAGD2_HUMAN | MAGED2 | 64.91 | 0 | 8 |
| Q9Y3Y2_CHTOP_HUMAN | CHTOP | 26.38 | 0 | 8 |
| P38159_RBMX_HUMAN | RBMX | 42.31 | 1 | 8 |
| P08107_HSP71_HUMAN | HSPA1A | 70.01 | 2 | 8 |
| P62753_RS6_HUMAN | RPS6 | 28.66 | 2 | 8 |
| Q96PK6_RBM14_HUMAN | RBM14 | 69.45 | 2 | 8 |
| P62269_RS18_HUMAN | RPS18 | 17.71 | 3 | 8 |
| Q9H4B7_TBB1_HUMAN | TUBB1 | 50.29 | 4 | 8 |
| Q14247_SRC8_HUMAN | CTTN | 61.55 | 6 | 8 |
| P21333_FLNA_HUMAN | FLNA | 280.56 | 46 | 8 |
| O75569_PRKRA_HUMAN | PRKRA | 34.38 | 0 | 7 |
| O75934_SPF27_HUMAN | BCAS2 | 26.11 | 0 | 7 |
| P62263_RS14_HUMAN | RPS14 | 16.26 | 0 | 7 |
| Q02878_RL6_HUMAN | RPL6 | 32.71 | 0 | 7 |
| Q15427_SF3B4_HUMAN | SF3B4 | 44.36 | 0 | 7 |
| Q16629_SRSF7_HUMAN | SRSF7 | 27.35 | 0 | 7 |
| Q9BUQ8_DDX23_HUMAN | DDX23 | 95.52 | 0 | 7 |
| Q9H0D6_XRN2_HUMAN | XRN2 | 108.51 | 0 | 7 |
| Q9NW64_RBM22_HUMAN | RBM22 | 46.87 | 0 | 7 |
| Q9Y4Z0_LSM4_HUMAN | LSM4 | 15.34 | 0 | 7 |
| Q9Y6M1_IF2B2_HUMAN | IGF2BP2 | 66.08 | 0 | 7 |
| Q13242_SRSF9_HUMAN | SRSF9 | 25.53 | 1 | 7 |
| Q9Y3B4_SF3B6_HUMAN | SF3B6 | 14.58 | 1 | 7 |
| P40926_MDHM_HUMAN | MDH2 | 35.48 | 2 | 7 |
| P00558_PGK1_HUMAN | PGK1 | 44.59 | 3 | 7 |
| P62244_RS15A_HUMAN | RPS15A | 14.83 | 3 | 7 |
| P62249_RS16_HUMAN | RPS16 | 16.44 | 3 | 7 |
| P00338_LDHA_HUMAN | LDHA | 36.67 | 5 | 7 |
| P33778_H2B1B_HUMAN | HIST1H2BB | 13.94 | 5 | 7 |
| Q58FF7_H90B3_HUMAN | HSP90AB3P | 68.28 | 5 | 7 |
| P07195_LDHB_HUMAN | LDHB | 36.62 | 7 | 7 |
| P30876_RPB2_HUMAN | POLR2B | 133.81 | 7 | 7 |
| P54652_HSP72_HUMAN | HSPA2 | 69.98 | 7 | 7 |
| P07900_HS90A_HUMAN | HSP90AA1 | 84.61 | 8 | 7 |
| Q99873_ANM1_HUMAN | PRMT1 | 41.49 | 11 | 7 |
| O43707_ACTN4_HUMAN | ACTN4 | 104.79 | 43 | 7 |
| P12814_ACTN1_HUMAN | ACTN1 | 102.99 | 59 | 7 |
| A8MWD9_RUXGL_HUMAN | SNRPGP15 | 8.54 | 0 | 6 |
| O75494_SRS10_HUMAN | SRSF10 | 31.28 | 0 | 6 |
| O95926_SYF2_HUMAN | SYF2 | 28.7 | 0 | 6 |
| P09651_ROA1_HUMAN | HNRNPA1 | 38.72 | 0 | 6 |
| P39023_RL3_HUMAN | RPL3 | 46.08 | 0 | 6 |
| P41227_NAA10_HUMAN | NAA10 | 26.44 | 0 | 6 |
| P48634_PRC2A_HUMAN | PRRC2A | 228.72 | 0 | 6 |
| P61247_RS3A_HUMAN | RPS3A | 29.93 | 0 | 6 |
| P62424_RL7A_HUMAN | RPL7A | 29.98 | 0 | 6 |
| Q1KMD3_HNRL2_HUMAN | HNRNPUL2 | 85.05 | 0 | 6 |
| Q4G0J3_LARP7_HUMAN | LARP7 | 66.86 | 0 | 6 |
| Q99729_ROAA_HUMAN | HNRNPAB | 36.2 | 0 | 6 |
| Q9NZB2_F120A_HUMAN | FAM120A | 121.81 | 0 | 6 |
| Q9UNP9_PPIE_HUMAN | PPIE | 33.41 | 0 | 6 |
| Q9Y333_LSM2_HUMAN | LSM2 | 10.83 | 0 | 6 |
| P36578_RL4_HUMAN | RPL4 | 47.67 | 1 | 6 |
| P84098_RL19_HUMAN | RPL19 | 23.45 | 1 | 6 |
| B4DY08_B4DY08_HUMAN | HNRNPC | 31.95 | 2 | 6 |
| O15523_DDX3Y_HUMAN | DDX3Y | 73.11 | 2 | 6 |
| P23526_SAHH_HUMAN | AHCY | 47.69 | 2 | 6 |
| P62304_RUXE_HUMAN | SNRPE | 10.8 | 3 | 6 |
| P19338_NUCL_HUMAN | NCL | 76.57 | 4 | 6 |
| P26373_RL13_HUMAN | RPL13 | 24.25 | 4 | 6 |
| Q15084_PDIA6_HUMAN | PDIA6 | 48.09 | 4 | 6 |
| Q16695_H31T_HUMAN | HIST3H3 | 15.5 | 4 | 6 |
| Q9BQA1_MEP50_HUMAN | WDR77 | 36.7 | 4 | 6 |
| Q9BXP5_SRRT_HUMAN | SRRT | 100.6 | 4 | 6 |
| P14625_ENPL_HUMAN | HSP90B1 | 92.41 | 5 | 6 |
| P08238_HS90B_HUMAN | HSP90AB1 | 83.21 | 7 | 6 |
| P54886_P5CS_HUMAN | ALDH18A1 | 87.25 | 9 | 6 |
| Q9BYX7_ACTBM_HUMAN | POTEKP | 41.99 | 22 | 6 |
| O15042_SR140_HUMAN | U2SURP | 118.22 | 0 | 5 |
| O43660_PLRG1_HUMAN | PLRG1 | 57.16 | 0 | 5 |
| O60828_PQBP1_HUMAN | PQBP1 | 30.45 | 0 | 5 |
| O75489_NDUS3_HUMAN | NDUFS3 | 30.22 | 0 | 5 |
| O95391_SLU7_HUMAN | SLU7 | 68.34 | 0 | 5 |
| O95872_GPAN1_HUMAN | GPANK1 | 39.29 | 0 | 5 |
| P16989_YBOX3_HUMAN | YBX3 | 40.07 | 0 | 5 |
| P31689_DNJA1_HUMAN | DNAJA1 | 44.84 | 0 | 5 |
| P34897_GLYM_HUMAN | SHMT2 | 55.96 | 0 | 5 |
| P39019_RS19_HUMAN | RPS19 | 16.05 | 0 | 5 |
| P41223_BUD31_HUMAN | BUD31 | 16.99 | 0 | 5 |
| P55081_MFAP1_HUMAN | MFAP1 | 51.93 | 0 | 5 |
| Q10570_CPSF1_HUMAN | CPSF1 | 160.78 | 0 | 5 |
| Q15637_SF01_HUMAN | SF1 | 68.29 | 0 | 5 |
| Q86V81_THOC4_HUMAN | ALYREF | 26.87 | 0 | 5 |
| Q9HCG8_CWC22_HUMAN | CWC22 | 105.4 | 0 | 5 |
| Q9P013_CWC15_HUMAN | CWC15 | 26.61 | 0 | 5 |
| Q9UH17_ABC3B_HUMAN | APOBEC3B | 45.89 | 0 | 5 |
| Q9Y3C6_PPIL1_HUMAN | PPIL1 | 18.23 | 0 | 5 |
| Q9Y5A9_YTHD2_HUMAN | YTHDF2 | 62.3 | 0 | 5 |
| P02786_TFR1_HUMAN | TFRC | 84.82 | 1 | 5 |
| P16403_H12_HUMAN | HIST1H1C | 21.35 | 1 | 5 |
| P27708_PYR1_HUMAN | CAD | 242.83 | 1 | 5 |
| P35232_PHB_HUMAN | PHB | 29.79 | 1 | 5 |
| Q09028_RBBP4_HUMAN | RBBP4 | 47.63 | 1 | 5 |
| Q13148_TADBP_HUMAN | TARDBP | 44.71 | 1 | 5 |
| Q7KZ85_SPT6H_HUMAN | SUPT6H | 198.95 | 1 | 5 |
| P48643_TCPE_HUMAN | CCT5 | 59.63 | 2 | 5 |
| P04350_TBB4A_HUMAN | TUBB4A | 49.55 | 3 | 5 |
| P62241_RS8_HUMAN | RPS8 | 24.19 | 3 | 5 |
| P63173_RL38_HUMAN | RPL38 | 8.21 | 3 | 5 |
| Q14204_DYHC1_HUMAN | DYNC1H1 | 532.07 | 3 | 5 |
| P22314_UBA1_HUMAN | UBA1 | 117.77 | 4 | 5 |
| P46821_MAP1B_HUMAN | MAP1B | 270.47 | 4 | 5 |
| Q14257_RCN2_HUMAN | RCN2 | 36.85 | 4 | 5 |
| Q9Y265_RUVB1_HUMAN | RUVBL1 | 50.2 | 4 | 5 |
| O43175_SERA_HUMAN | PHGDH | 56.61 | 5 | 5 |
| P55884_EIF3B_HUMAN | EIF3B | 92.42 | 6 | 5 |
| Q9UDY2_ZO2_HUMAN | TJP2 | 133.88 | 6 | 5 |
| P24928_RPB1_HUMAN | POLR2A | 217.04 | 8 | 5 |
| Q9HCD5_NCOA5_HUMAN | NCOA5 | 65.5 | 8 | 5 |
| Q00610_CLH1_HUMAN | CLTC | 191.49 | 13 | 5 |
| P61158_ARP3_HUMAN | ACTR3 | 47.34 | 15 | 5 |
| P47756_CAPZB_HUMAN | CAPZB | 31.33 | 24 | 5 |
| P00966_ASSY_HUMAN | ASS1 | 46.5 | 0 | 4 |
| P02452_CO1A1_HUMAN | COL1A1 | 138.86 | 0 | 4 |
| P12004_PCNA_HUMAN | PCNA | 28.75 | 0 | 4 |
| P29374_ARI4A_HUMAN | ARID4A | 142.66 | 0 | 4 |
| P32969_RL9_HUMAN | RPL9 | 21.85 | 0 | 4 |
| P33991_MCM4_HUMAN | MCM4 | 96.5 | 0 | 4 |
| P40939_ECHA_HUMAN | HADHA | 82.95 | 0 | 4 |
| P42166_LAP2A_HUMAN | TMPO | 75.45 | 0 | 4 |
| P52292_IMA1_HUMAN | KPNA2 | 57.83 | 0 | 4 |
| P57721_PCBP3_HUMAN | PCBP3 | 39.44 | 0 | 4 |
| P62314_SMD1_HUMAN | SNRPD1 | 13.27 | 0 | 4 |
| P62917_RL8_HUMAN | RPL8 | 28.01 | 0 | 4 |
| P82650_RT22_HUMAN | MRPS22 | 41.25 | 0 | 4 |
| Q07021_C1QBP_HUMAN | C1QBP | 31.34 | 0 | 4 |
| Q14157_UBP2L_HUMAN | UBAP2L | 114.47 | 0 | 4 |
| Q14576_ELAV3_HUMAN | ELAVL3 | 39.52 | 0 | 4 |
| Q15233_NONO_HUMAN | NONO | 54.2 | 0 | 4 |
| Q15424_SAFB1_HUMAN | SAFB | 102.58 | 0 | 4 |
| Q3KQU3_MA7D1_HUMAN | MAP7D1 | 92.76 | 0 | 4 |
| Q53GS9_SNUT2_HUMAN | USP39 | 65.34 | 0 | 4 |
| Q6UX04_CWC27_HUMAN | CWC27 | 53.81 | 0 | 4 |
| Q8IWZ8_SUGP1_HUMAN | SUGP1 | 72.43 | 0 | 4 |
| Q96DF8_DGC14_HUMAN | DGCR14 | 52.54 | 0 | 4 |
| Q96DH6_MSI2H_HUMAN | MSI2 | 35.17 | 0 | 4 |
| Q9H2U1_DHX36_HUMAN | DHX36 | 114.69 | 0 | 4 |
| Q9P2K5_MYEF2_HUMAN | MYEF2 | 64.08 | 0 | 4 |
| Q9ULX6_AKP8L_HUMAN | AKAP8L | 71.6 | 0 | 4 |
| Q9Y3I0_RTCB_HUMAN | RTCB | 55.17 | 0 | 4 |
| Q9Y5B9_SP16H_HUMAN | SUPT16H | 119.84 | 0 | 4 |
| O14979_HNRDL_HUMAN | HNRNPDL | 46.41 | 1 | 4 |
| P08195_4F2_HUMAN | SLC3A2 | 67.95 | 1 | 4 |
| P15880_RS2_HUMAN | RPS2 | 31.3 | 1 | 4 |
| P17844_DDX5_HUMAN | DDX5 | 69.1 | 1 | 4 |
| P31150_GDIA_HUMAN | GDI1 | 50.55 | 1 | 4 |
| P63104_1433Z_HUMAN | YWHAZ | 27.73 | 1 | 4 |
| P83731_RL24_HUMAN | RPL24 | 17.77 | 1 | 4 |
| Q13595_TRA2A_HUMAN | TRA2A | 32.67 | 1 | 4 |
| Q15293_RCN1_HUMAN | RCN1 | 38.87 | 1 | 4 |
| Q99714_HCD2_HUMAN | HSD17B10 | 26.91 | 1 | 4 |
| O000303_EIF3F_HUMAN | EIF3F | 37.54 | 2 | 4 |
| O43809_CPSF5_HUMAN | NUDT21 | 26.21 | 2 | 4 |
| Q07020_RL18_HUMAN | RPL18 | 21.62 | 2 | 4 |
| Q14697_GANAB_HUMAN | GANAB | 106.81 | 2 | 4 |
| Q92598_HS105_HUMAN | HSPH1 | 96.8 | 2 | 4 |
| P04075_ALDOA_HUMAN | ALDOA | 39.4 | 3 | 4 |
| P06744_G6PI_HUMAN | GPI | 63.11 | 3 | 4 |
| P26641_EF1G_HUMAN | EEF1G | 50.09 | 3 | 4 |
| Q58FF8_H90B2_HUMAN | HSP90AB2P | 44.32 | 3 | 4 |
| Q8NHW5_RLA0L_HUMAN | RPLP0P6 | 34.34 | 3 | 4 |
| P08865_RSSA_HUMAN | RPSA | 32.83 | 4 | 4 |
| P53396_ACLY_HUMAN | ACLY | 120.76 | 4 | 4 |
| Q06830_PRDX1_HUMAN | PRDX1 | 22.1 | 5 | 4 |
| Q96QV6_H2A1A_HUMAN | HIST1H2AA | 14.22 | 6 | 4 |
| P47755_CAZA2_HUMAN | CAPZA2 | 32.93 | 17 | 4 |
| O14556_G3PT_HUMAN | GAPDHS | 44.47 | 0 | 3 |
| O14929_HAT1_HUMAN | HAT1 | 49.48 | 0 | 3 |
| O15226_NKRF_HUMAN | NKRF | 77.62 | 0 | 3 |
| O43242_PSMD3_HUMAN | PSMD3 | 60.94 | 0 | 3 |
| O75940_SPF30_HUMAN | SMNDC1 | 26.69 | 0 | 3 |
| O95777_LSM8_HUMAN | LSM8 | 10.4 | 0 | 3 |
| O95831_AIFM1_HUMAN | AIFM1 | 66.86 | 0 | 3 |
| P04792_HSPB1_HUMAN | HSPB1 | 22.77 | 0 | 3 |
| P04843_RPN1_HUMAN | RPN1 | 68.53 | 0 | 3 |
| P05388_RLA0_HUMAN | RPLP0 | 34.25 | 0 | 3 |
| P09012_SNRPA_HUMAN | SNRPA | 31.26 | 0 | 3 |
| P19474_RO52_HUMAN | TRIM21 | 54.14 | 0 | 3 |
| P21796_VDAC1_HUMAN | VDAC1 | 30.75 | 0 | 3 |
| P22695_QCR2_HUMAN | UQCRC2 | 48.41 | 0 | 3 |
| P23246_SFPQ_HUMAN | SFPQ | 76.1 | 0 | 3 |
| P27694_RFA1_HUMAN | RPA1 | 68.1 | 0 | 3 |
| P42766_RL35_HUMAN | RPL35 | 14.54 | 0 | 3 |
| P43686_PRS6B_HUMAN | PSMC4 | 47.34 | 0 | 3 |
| P49756_RBM25_HUMAN | RBM25 | 100.12 | 0 | 3 |
| P50991_TCPD_HUMAN | CCT4 | 57.89 | 0 | 3 |
| P55060_XPO2_HUMAN | CSE1L | 110.35 | 0 | 3 |
| P61026_RAB10_HUMAN | RAB10 | 22.53 | 0 | 3 |
| P62191_PRS4_HUMAN | PSMC1 | 49.15 | 0 | 3 |
| P62277_RS13_HUMAN | RPS13 | 17.21 | 0 | 3 |
| P62306_RUXF_HUMAN | SNRPF | 9.72 | 0 | 3 |
| P62701_RS4X_HUMAN | RPS4X | 29.58 | 0 | 3 |
| P62906_RL10A_HUMAN | RPL10A | 24.82 | 0 | 3 |
| P84103_SRSF3_HUMAN | SRSF3 | 19.32 | 0 | 3 |
| Q00577_PURA_HUMAN | PURA | 34.89 | 0 | 3 |
| Q08170_SRSF4_HUMAN | SRSF4 | 56.65 | 0 | 3 |
| Q14151_SAFB2_HUMAN | SAFB2 | 107.41 | 0 | 3 |
| Q14671_PUM1_HUMAN | PUM1 | 126.39 | 0 | 3 |
| Q16531_DDB1_HUMAN | DDB1 | 126.89 | 0 | 3 |
| Q16630_CPSF6_HUMAN | CPSF6 | 59.17 | 0 | 3 |
| Q6NYC1_JMJD6_HUMAN | JMJD6 | 46.43 | 0 | 3 |
| Q6NZY4_ZCHC8_HUMAN | ZCCHC8 | 78.53 | 0 | 3 |
| Q8IXT5_RB12B_HUMAN | RBM12B | 118.03 | 0 | 3 |
| Q8TAA3_PSA7L_HUMAN | PSMA8 | 28.51 | 0 | 3 |
| Q8WUD4_CCD12_HUMAN | CCDC12 | 19.17 | 0 | 3 |
| Q8WUQ7_CATIN_HUMAN | CACTIN | 88.65 | 0 | 3 |
| Q96A08_H2B1A_HUMAN | HIST1H2BA | 14.16 | 0 | 3 |
| Q96BP3_PPWD1_HUMAN | PPWD1 | 73.53 | 0 | 3 |
| Q96KR1_ZFR_HUMAN | ZFR | 116.94 | 0 | 3 |
| Q99962_SH3G2_HUMAN | SH3GL2 | 39.94 | 0 | 3 |
| Q9BQ04_RBM4B_HUMAN | RBM4B | 40.12 | 0 | 3 |
| Q9BYJ9_YTHD1_HUMAN | YTHDF1 | 60.84 | 0 | 3 |
| Q9NWB1_RFOX1_HUMAN | RBFOX1 | 42.76 | 0 | 3 |
| Q9UKV3_ACINU_HUMAN | ACIN1 | 151.77 | 0 | 3 |
| Q9ULR0_ISY1_HUMAN | ISY1 | 32.97 | 0 | 3 |
| Q9UQ35_SRRM2_HUMAN | SRRM2 | 299.44 | 0 | 3 |
| Q9UQ80_PA2G4_HUMAN | PA2G4 | 43.76 | 0 | 3 |
| Q9Y262_EIF3L_HUMAN | EIF3L | 66.68 | 0 | 3 |
| Q9Y2Z0_SUGT1_HUMAN | SUGT1 | 41 | 0 | 3 |
| Q9Y3X0_CCDC9_HUMAN | CCDC9 | 59.67 | 0 | 3 |
| O75083_WDR1_HUMAN | WDR1 | 66.15 | 1 | 3 |
| P08243_ASNS_HUMAN | ASNS | 64.33 | 1 | 3 |
| P27635_RL10_HUMAN | RPL10 | 24.59 | 1 | 3 |
| P35268_RL22_HUMAN | RPL22 | 14.78 | 1 | 3 |
| P45880_VDAC2_HUMAN | VDAC2 | 31.55 | 1 | 3 |
| P52756_RBM5_HUMAN | RBM5 | 92.1 | 1 | 3 |
| P61313_RL15_HUMAN | RPL15 | 24.13 | 1 | 3 |
| P61353_RL27_HUMAN | RPL27 | 15.79 | 1 | 3 |
| Q14103_HNRPD_HUMAN | HNRNPD | 38.41 | 1 | 3 |
| Q86VP6_CAND1_HUMAN | CAND1 | 136.29 | 1 | 3 |
| Q9Y3F4_STRAP_HUMAN | STRAP | 38.41 | 1 | 3 |
| G3V0J0_G3V0J0_HUMAN | FMR1 | 66.74 | 2 | 3 |
| P06753_TPM3_HUMAN | TPM3 | 32.93 | 2 | 3 |
| P12235_ADT1_HUMAN | SLC25A4 | 33.04 | 2 | 3 |
| P27824_CALX_HUMAN | CANX | 67.53 | 2 | 3 |
| P33992_MCM5_HUMAN | MCM5 | 82.23 | 2 | 3 |
| P40227_TCPZ_HUMAN | CCT6A | 57.99 | 2 | 3 |
| P41252_SYIC_HUMAN | IARS | 144.41 | 2 | 3 |
| P49750_YLPM1_HUMAN | YLPM1 | 219.85 | 2 | 3 |
| P60866_RS20_HUMAN | RPS20 | 13.36 | 2 | 3 |
| P62318_SMD3_HUMAN | SNRPD3 | 13.91 | 2 | 3 |
| P62851_RS25_HUMAN | RPS25 | 13.73 | 2 | 3 |
| P62987_RL40_HUMAN | UBA52 | 14.72 | 2 | 3 |
| P68366_TBA4A_HUMAN | TUBA4A | 49.89 | 2 | 3 |
| Q02539_H11_HUMAN | HIST1H1A | 21.83 | 2 | 3 |
| Q9H0G5_NSRP1_HUMAN | NSRP1 | 66.35 | 2 | 3 |
| O75367_H2AY_HUMAN | H2AFY | 39.59 | 3 | 3 |
| P05141_ADT2_HUMAN | SLC25A5 | 32.83 | 3 | 3 |
| P09493_TPM1_HUMAN | TPM1 | 32.69 | 3 | 3 |
| P20700_LMNB1_HUMAN | LMNB1 | 66.37 | 3 | 3 |
| P62826_RAN_HUMAN | RAN | 24.41 | 3 | 3 |
| Q9BQ39_DDX50_HUMAN | DDX50 | 82.51 | 3 | 3 |
| P11586_C1TC_HUMAN | MTHFD1 | 101.5 | 4 | 3 |
| P32119_PRDX2_HUMAN | PRDX2 | 21.88 | 4 | 3 |
| P25789_PSA4_HUMAN | PSMA4 | 29.47 | 5 | 3 |
| P31946_1433B_HUMAN | YWHAB | 28.06 | 5 | 3 |
| Q9Y657_SPIN1_HUMAN | SPIN1 | 29.58 | 7 | 3 |
| Q8NEY8_PPHLN_HUMAN | PPHLN1 | 52.71 | 9 | 3 |
| Q5JUX0_SPIN3_HUMAN | SPIN3 | 29.19 | 10 | 3 |
| Q9NZR1_TMOD2_HUMAN | TMOD2 | 39.57 | 23 | 3 |
| Q9NYL9_TMOD3_HUMAN | TMOD3 | 39.57 | 27 | 3 |
| Q9UHB6_LIMA1_HUMAN | LIMA1 | 85.17 | 55 | 3 |
| O43447_PPIH_HUMAN | PPIH | 19.2 | 0 | 2 |
| O60832_DKC1_HUMAN | DKC1 | 57.64 | 0 | 2 |
| O60884_DNJA2_HUMAN | DNAJA2 | 45.72 | 0 | 2 |
| O75390_CISY_HUMAN | CS | 51.68 | 0 | 2 |
| O75400_PR40A_HUMAN | PRPF40A | 108.74 | 0 | 2 |
| O95793_STAU1_HUMAN | STAU1 | 63.14 | 0 | 2 |
| P00367_DHE3_HUMAN | GLUD1 | 61.36 | 0 | 2 |
| P00505_AATM_HUMAN | GOT2 | 47.49 | 0 | 2 |
| P05387_RLA2_HUMAN | RPLP2 | 11.66 | 0 | 2 |
| P09234_RU1C_HUMAN | SNRPC | 17.38 | 0 | 2 |
| P17612_KAPCA_HUMAN | PRKACA | 40.56 | 0 | 2 |
| P18621_RL17_HUMAN | RPL17 | 21.38 | 0 | 2 |
| P22234_PUR6_HUMAN | PAICS | 47.05 | 0 | 2 |
| P23381_SYWC_HUMAN | WARS | 53.13 | 0 | 2 |
| P24534_EF1B_HUMAN | EEF1B2 | 24.75 | 0 | 2 |
| P26639_SYTC_HUMAN | TARS | 83.38 | 0 | 2 |
| P28066_PSA5_HUMAN | PSMA5 | 26.39 | 0 | 2 |
| P30041_PRDX6_HUMAN | PRDX6 | 25.02 | 0 | 2 |
| P34896_GLYC_HUMAN | SHMT1 | 53.05 | 0 | 2 |
| P35637_FUS_HUMAN | FUS | 53.39 | 0 | 2 |
| P36957_ODO2_HUMAN | DLST | 48.72 | 0 | 2 |
| P40429_RL13A_HUMAN | RPL13A | 23.56 | 0 | 2 |
| P46777_RL5_HUMAN | RPL5 | 34.34 | 0 | 2 |
| P46778_RL21_HUMAN | RPL21 | 18.55 | 0 | 2 |
| P51398_RT29_HUMAN | DAP3 | 45.54 | 0 | 2 |
| P51571_SSRD_HUMAN | SSR4 | 18.99 | 0 | 2 |
| P54136_SYRC_HUMAN | RARS | 75.33 | 0 | 2 |
| P55209_NP1L1_HUMAN | NAP1L1 | 45.35 | 0 | 2 |
| P62310_LSM3_HUMAN | LSM3 | 11.84 | 0 | 2 |
| P62312_LSM6_HUMAN | LSM6 | 9.12 | 0 | 2 |
| P62750_RL23A_HUMAN | RPL23A | 17.68 | 0 | 2 |
| P62847_RS24_HUMAN | RPS24 | 15.41 | 0 | 2 |
| P67775_PP2AA_HUMAN | PPP2CA | 35.57 | 0 | 2 |
| Q01081_U2AF1_HUMAN | U2AF1 | 27.85 | 0 | 2 |
| Q07666_KHDR1_HUMAN | KHDRBS1 | 48.2 | 0 | 2 |
| Q13185_CBX3_HUMAN | CBX3 | 20.8 | 0 | 2 |
| Q13188_STK3_HUMAN | STK3 | 56.26 | 0 | 2 |
| Q13247_SRSF6_HUMAN | SRSF6 | 39.56 | 0 | 2 |
| Q14194_DPYL1_HUMAN | CRMP1 | 62.14 | 0 | 2 |
| Q14683_SMC1A_HUMAN | SMC1A | 143.14 | 0 | 2 |
| Q58FG0_HS905_HUMAN | HSP90AA5P | 38.71 | 0 | 2 |
| Q659C4_LAR1B_HUMAN | LARP1B | 105.26 | 0 | 2 |
| Q6NVV1_R13P3_HUMAN | RPL13AP3 | 12.13 | 0 | 2 |
| Q6UN15_FIP1_HUMAN | FIP1L1 | 66.49 | 0 | 2 |
| Q71RC2_LARP4_HUMAN | LARP4 | 80.55 | 0 | 2 |
| Q7L2J0_MEPCE_HUMAN | MEPCE | 74.31 | 0 | 2 |
| Q8IUH3_RBM45_HUMAN | RBM45 | 53.47 | 0 | 2 |
| Q8IYB3_SRRM1_HUMAN | SRRM1 | 102.27 | 0 | 2 |
| Q8TB72_PUM2_HUMAN | PUM2 | 114.14 | 0 | 2 |
| Q8WXX5_DNJC9_HUMAN | DNAJC9 | 29.89 | 0 | 2 |
| Q8WYA6_CTBL1_HUMAN | CTNNBL1 | 65.13 | 0 | 2 |
| Q92917_GPKOW_HUMAN | GPKOW | 52.2 | 0 | 2 |
| Q93009_UBP7_HUMAN | USP7 | 128.22 | 0 | 2 |
| Q96K80_ZC3HA_HUMAN | ZC3H10 | 46.02 | 0 | 2 |
| Q96SI9_STRBP_HUMAN | STRBP | 73.61 | 0 | 2 |
| Q99623_PHB2_HUMAN | PHB2 | 33.28 | 0 | 2 |
| Q99700_ATX2_HUMAN | ATXN2 | 140.2 | 0 | 2 |
| Q9BWF3_RBM4_HUMAN | RBM4 | 40.29 | 0 | 2 |
| Q9H2H8_PPIL3_HUMAN | PPIL3 | 18.14 | 0 | 2 |
| Q9H5Z1_DHX35_HUMAN | DHX35 | 78.86 | 0 | 2 |
| Q9NQ39_RS10L_HUMAN | RPS10P5 | 20.11 | 0 | 2 |
| Q9NR56_MBNL1_HUMAN | MBNL1 | 41.79 | 0 | 2 |
| Q9NUD5_ZCHC3_HUMAN | ZCCHC3 | 43.59 | 0 | 2 |
| Q9NW82_WDR70_HUMAN | WDR70 | 73.16 | 0 | 2 |
| Q9NX05_F120C_HUMAN | FAM120C | 120.51 | 0 | 2 |
| Q9P0M6_H2AW_HUMAN | H2AFY2 | 40.03 | 0 | 2 |
| Q9UBS4_DJB11_HUMAN | DNAJB11 | 40.49 | 0 | 2 |
| Q9UNX3_RL26L_HUMAN | RPL26L1 | 17.25 | 0 | 2 |
| Q9Y5B6_PAXB1_HUMAN | PAXBP1 | 104.74 | 0 | 2 |
| E9PAV3_NACAM_HUMAN | NACA | 205.29 | 1 | 2 |
| P0CW22_RS17L_HUMAN | RPS17L | 15.54 | 1 | 2 |
| P13804_ETFA_HUMAN | ETFA | 35.06 | 1 | 2 |
| P31153_METK2_HUMAN | MAT2A | 43.63 | 1 | 2 |
| P40925_MDHC_HUMAN | MDH1 | 36.4 | 1 | 2 |
| P41250_SYG_HUMAN | GARS | 83.11 | 1 | 2 |
| P46783_RS10_HUMAN | RPS10 | 18.89 | 1 | 2 |
| P53621_COPA_HUMAN | COPA | 138.26 | 1 | 2 |
| P62807_H2B1C_HUMAN | HIST1H2BC | 13.9 | 1 | 2 |
| P62899_RL31_HUMAN | RPL31 | 14.45 | 1 | 2 |
| P63244_GBLP_HUMAN | GNB2L1 | 35.05 | 1 | 2 |
| Q15758_AAAT_HUMAN | SLC1A5 | 56.56 | 1 | 2 |
| Q71UI9_H2AV_HUMAN | H2AFV | 13.5 | 1 | 2 |
| Q99613_EIF3C_HUMAN | EIF3C | 105.28 | 1 | 2 |
| Q99832_TCPH_HUMAN | CCT7 | 59.33 | 1 | 2 |
| P19387_RPB3_HUMAN | POLR2C | 31.42 | 2 | 2 |
| P20618_PSB1_HUMAN | PSMB1 | 26.47 | 2 | 2 |
| P23284_PPIB_HUMAN | PPIB | 23.73 | 2 | 2 |
| P30050_RL12_HUMAN | RPL12 | 17.81 | 2 | 2 |
| P62195_PRS8_HUMAN | PSMC5 | 45.6 | 2 | 2 |
| P62491_RB11A_HUMAN | RAB11A | 24.38 | 2 | 2 |
| P62913_RL11_HUMAN | RPL11 | 20.24 | 2 | 2 |
| P84077_ARF1_HUMAN | ARF1 | 20.68 | 2 | 2 |
| Q08945_SSRP1_HUMAN | SSRP1 | 81.02 | 2 | 2 |
| Q71UM5_RS27L_HUMAN | RPS27L | 9.47 | 2 | 2 |
| Q9P2J5_SYLC_HUMAN | LARS | 134.38 | 2 | 2 |
| P62258_1433E_HUMAN | YWHAE | 29.16 | 3 | 2 |
| Q13162_PRDX4_HUMAN | PRDX4 | 30.52 | 3 | 2 |
| Q14568_HS902_HUMAN | HSP90AA2 | 39.34 | 3 | 2 |
| Q5HYB6_Q5HYB6_HUMAN | DKFZp686J1372 | 27.16 | 3 | 2 |
| P05023_AT1A1_HUMAN | ATP1A1 | 112.82 | 4 | 2 |
| P07237_PDIA1_HUMAN | P4HB | 57.08 | 4 | 2 |
| P29401_TKT_HUMAN | TKT | 67.83 | 4 | 2 |
| Q99865_SPI2A_HUMAN | SPIN2A | 29.17 | 4 | 2 |
| P25311_ZA2G_HUMAN | AZGP1 | 34.24 | 5 | 2 |
| P55072_TERA_HUMAN | VCP | 89.27 | 5 | 2 |
| P07196_NFL_HUMAN | NEFL | 61.48 | 6 | 2 |
| P62136_PP1A_HUMAN | PPP1CA | 37.49 | 6 | 2 |
| O15145_ARPC3_HUMAN | ARPC3 | 20.53 | 8 | 2 |
| P15311_EZRI_HUMAN | EZR | 69.37 | 10 | 2 |
| Q7Z353_HDX_HUMAN | HDX | 77.16 | 31 | 2 |
| P35580_MYH10_HUMAN | MYH10 | 228.86 | 37 | 2 |
| A6NHL2_TBAL3_HUMAN | TUBAL3 | 49.88 | 0 | 1 |
| A6NMZ7_CO6A6_HUMAN | COL6A6 | 247.02 | 0 | 1 |
| B7Z645_B7Z645_HUMAN | SYNCRIP | 52.01 | 0 | 1 |
| C9JUF0_C9JUF0_HUMAN | EIF4A2 | 6.32 | 0 | 1 |
| D6RBZ0_D6RBZ0_HUMAN | HNRNPAB | 35.66 | 0 | 1 |
| E9PCK9_E9PCK9_HUMAN | MLIP | 59.08 | 0 | 1 |
| F8VRH0_F8VRH0_HUMAN | PCBP2 | 32.02 | 0 | 1 |
| G3V542_G3V542_HUMAN | TUBB3 | 4.97 | 0 | 1 |
| H7BZJ3_H7BZJ3_HUMAN | PDIA3 | 13.51 | 0 | 1 |
| H7C1M2_H7C1M2_HUMAN | SON | 159.92 | 0 | 1 |
| O00231_PSD11_HUMAN | PSMD11 | 47.43 | 0 | 1 |
| O00487_PSDE_HUMAN | PSMD14 | 34.55 | 0 | 1 |
| O14980_XPO1_HUMAN | XPO1 | 123.31 | 0 | 1 |
| O14983_AT2A1_HUMAN | ATP2A1 | 110.18 | 0 | 1 |
| O15234_CASC3_HUMAN | CASC3 | 76.23 | 0 | 1 |
| O15355_PPM1G_HUMAN | PPM1G | 59.23 | 0 | 1 |
| O15371_EIF3D_HUMAN | EIF3D | 63.93 | 0 | 1 |
| O15372_EIF3H_HUMAN | EIF3H | 39.91 | 0 | 1 |
| O43251_RFOX2_HUMAN | RBFOX2 | 41.35 | 0 | 1 |
| O43347_MSI1H_HUMAN | MSI1 | 39.1 | 0 | 1 |
| O43684_BUB3_HUMAN | BUB3 | 37.13 | 0 | 1 |
| O43776_SYNC_HUMAN | NARS | 62.9 | 0 | 1 |
| O43823_AKAP8_HUMAN | AKAP8 | 76.06 | 0 | 1 |
| O75417_DPOLQ_HUMAN | POLQ | 289.44 | 0 | 1 |
| O75531_BAF_HUMAN | BANF1 | 10.05 | 0 | 1 |
| O75554_WBP4_HUMAN | WBP4 | 42.48 | 0 | 1 |
| O75638_CTAG2_HUMAN | CTAG2 | 21.05 | 0 | 1 |
| O76021_RL1D1_HUMAN | RSL1D1 | 54.94 | 0 | 1 |
| O95299_NDUAA_HUMAN | NDUFA10 | 40.72 | 0 | 1 |
| O95433_AHSA1_HUMAN | AHSA1 | 38.25 | 0 | 1 |
| O95477_ABCA1_HUMAN | ABCA1 | 254.14 | 0 | 1 |
| O95672_ECEL1_HUMAN | ECEL1 | 87.74 | 0 | 1 |
| O95758_PTBP3_HUMAN | PTBP3 | 59.65 | 0 | 1 |
| O95816_BAG2_HUMAN | BAG2 | 23.76 | 0 | 1 |
| P02792_FRIL_HUMAN | FTL | 20.01 | 0 | 1 |
| P03989_1B27_HUMAN | HLA-B | 40.4 | 0 | 1 |
| P04181_OAT_HUMAN | OAT | 48.5 | 0 | 1 |
| P04818_TYSY_HUMAN | TYMS | 35.69 | 0 | 1 |
| P05067_A4_HUMAN | APP | 86.89 | 0 | 1 |
| P05198_IF2A_HUMAN | EIF2S1 | 36.09 | 0 | 1 |
| P08559_ODPA_HUMAN | PDHA1 | 43.27 | 0 | 1 |
| P08754_GNAI3_HUMAN | GNAI3 | 40.51 | 0 | 1 |
| P09960_LKHA4_HUMAN | LTA4H | 69.24 | 0 | 1 |
| P09972_ALDOC_HUMAN | ALDOC | 39.43 | 0 | 1 |
| P10515_ODP2_HUMAN | DLAT | 68.95 | 0 | 1 |
| P11177_ODPB_HUMAN | PDHB | 39.21 | 0 | 1 |
| P11233_RALA_HUMAN | RALA | 23.55 | 0 | 1 |
| P12236_ADT3_HUMAN | SLC25A6 | 32.85 | 0 | 1 |
| P13473_LAMP2_HUMAN | LAMP2 | 44.93 | 0 | 1 |
| P14406_CX7A2_HUMAN | COX7A2 | 9.39 | 0 | 1 |
| P18077_RL35A_HUMAN | RPL35A | 12.53 | 0 | 1 |
| P19367_HXK1_HUMAN | HK1 | 102.42 | 0 | 1 |
| P21281_VATB2_HUMAN | ATP6V1B2 | 56.46 | 0 | 1 |
| P25398_RS12_HUMAN | RPS12 | 14.51 | 0 | 1 |
| P29692_EF1D_HUMAN | EEF1D | 31.1 | 0 | 1 |
| P30443_1A01_HUMAN | HLA-A | 40.82 | 0 | 1 |
| P31941_ABC3A_HUMAN | APOBEC3A | 23 | 0 | 1 |
| P33240_CSTF2_HUMAN | CSTF2 | 60.92 | 0 | 1 |
| P34932_HSP74_HUMAN | HSPA4 | 94.27 | 0 | 1 |
| P35998_PRS7_HUMAN | PSMC2 | 48.6 | 0 | 1 |
| P37837_TALDO_HUMAN | TALDO1 | 37.52 | 0 | 1 |
| P38432_COIL_HUMAN | COIL | 62.57 | 0 | 1 |
| P40937_RFC5_HUMAN | RFC5 | 38.47 | 0 | 1 |
| P42167_LAP2B_HUMAN | TMPO | 50.64 | 0 | 1 |
| P42224_STAT1_HUMAN | STAT1 | 87.28 | 0 | 1 |
| P42677_RS27_HUMAN | RPS27 | 9.45 | 0 | 1 |
| P43034_LIS1_HUMAN | PAFAH1B1 | 46.61 | 0 | 1 |
| P43355_MAGA1_HUMAN | MAGEA1 | 34.32 | 0 | 1 |
| P46013_KI67_HUMAN | MKI67 | 358.47 | 0 | 1 |
| P46379_BAG6_HUMAN | BAG6 | 119.33 | 0 | 1 |
| P46782_RS5_HUMAN | RPS5 | 22.86 | 0 | 1 |
| P47914_RL29_HUMAN | RPL29 | 17.74 | 0 | 1 |
| P48047_ATPO_HUMAN | ATP5O | 23.26 | 0 | 1 |
| P48444_COPD_HUMAN | ARCN1 | 57.17 | 0 | 1 |
| P48735_IDHP_HUMAN | IDH2 | 50.88 | 0 | 1 |
| P49207_RL34_HUMAN | RPL34 | 13.28 | 0 | 1 |
| P49721_PSB2_HUMAN | PSMB2 | 22.82 | 0 | 1 |
| P52298_NCBP2_HUMAN | NCBP2 | 17.99 | 0 | 1 |
| P52565_GDIR1_HUMAN | ARHGDIA | 23.19 | 0 | 1 |
| P52815_RM12_HUMAN | MRPL12 | 21.33 | 0 | 1 |
| P55769_NH2L1_HUMAN | NHP2L1 | 14.16 | 0 | 1 |
| P56385_ATP5I_HUMAN | ATP5I | 7.93 | 0 | 1 |
| P56556_NDUA6_HUMAN | NDUFA6 | 17.86 | 0 | 1 |
| P60900_PSA6_HUMAN | PSMA6 | 27.38 | 0 | 1 |
| P61009_SPCS3_HUMAN | SPCS3 | 20.3 | 0 | 1 |
| P61019_RAB2A_HUMAN | RAB2A | 23.53 | 0 | 1 |
| P61088_UBE2N_HUMAN | UBE2N | 17.13 | 0 | 1 |
| P61513_RL37A_HUMAN | RPL37A | 10.27 | 0 | 1 |
| P61764_STXB1_HUMAN | STXBP1 | 67.53 | 0 | 1 |
| P62273_RS29_HUMAN | RPS29 | 6.67 | 0 | 1 |
| P62875_RPAB5_HUMAN | POLR2L | 7.64 | 0 | 1 |
| P62888_RL30_HUMAN | RPL30 | 12.78 | 0 | 1 |
| P68402_PA1B2_HUMAN | PAFAH1B2 | 25.55 | 0 | 1 |
| P82912_RT11_HUMAN | MRPS11 | 20.6 | 0 | 1 |
| P83876_TXN4A_HUMAN | TXNL4A | 16.78 | 0 | 1 |
| Q01130_SRSF2_HUMAN | SRSF2 | 25.46 | 0 | 1 |
| Q01844_EWS_HUMAN | EWSR1 | 68.44 | 0 | 1 |
| Q02543_RL18A_HUMAN | RPL18A | 20.75 | 0 | 1 |
| Q05519_SRS11_HUMAN | SRSF11 | 53.51 | 0 | 1 |
| Q06210_GFPT1_HUMAN | GFPT1 | 78.76 | 0 | 1 |
| Q08J23_NSUN2_HUMAN | NSUN2 | 86.42 | 0 | 1 |
| Q13200_PSMD2_HUMAN | PSMD2 | 100.14 | 0 | 1 |
| Q14331_FRG1_HUMAN | FRG1 | 29.15 | 0 | 1 |
| Q14444_CAPR1_HUMAN | CAPRIN1 | 78.32 | 0 | 1 |
| Q14566_MCM6_HUMAN | MCM6 | 92.83 | 0 | 1 |
| Q14980_NUMA1_HUMAN | NUMA1 | 238.12 | 0 | 1 |
| Q15008_PSMD6_HUMAN | PSMD6 | 45.5 | 0 | 1 |
| Q15057_ACAP2_HUMAN | ACAP2 | 87.97 | 0 | 1 |
| Q15369_ELOC_HUMAN | TCEB1 | 12.46 | 0 | 1 |
| Q16658_FSCN1_HUMAN | FSCN1 | 54.5 | 0 | 1 |
| Q16718_NDUA5_HUMAN | NDUFA5 | 13.45 | 0 | 1 |
| Q3MHD2_LSM12_HUMAN | LSM12 | 21.69 | 0 | 1 |
| Q5JNZ5_RS26L_HUMAN | RPS26P11 | 12.99 | 0 | 1 |
| Q5JVF3_PCID2_HUMAN | PCID2 | 46 | 0 | 1 |
| Q5VT06_CE350_HUMAN | CEP350 | 350.72 | 0 | 1 |
| Q5VU97_CAHD1_HUMAN | CACHD1 | 142.2 | 0 | 1 |
| Q5VWT5_CA168_HUMAN | C1orf168 | 82.02 | 0 | 1 |
| Q6P2E9_EDC4_HUMAN | EDC4 | 151.57 | 0 | 1 |
| Q6UB35_C1TM_HUMAN | MTHFD1L | 105.72 | 0 | 1 |
| Q6ZSN1_YI023_HUMAN | | 16.65 | 0 | 1 |
| Q7KZF4_SND1_HUMAN | SND1 | 101.93 | 0 | 1 |
| Q7RTV0_PHF5A_HUMAN | PHF5A | 12.4 | 0 | 1 |
| Q8N684_CPSF7_HUMAN | CPSF7 | 52.02 | 0 | 1 |
| Q8N6T0_CK080_HUMAN | C11orf80 | 74.59 | 0 | 1 |
| Q8NCA5_FA98A_HUMAN | FAM98A | 55.37 | 0 | 1 |
| Q8NEQ5_CA162_HUMAN | C1orf162 | 16.88 | 0 | 1 |
| Q8NI27_THOC2_HUMAN | THOC2 | 182.66 | 0 | 1 |
| Q8TBK6_ZCH10_HUMAN | ZCCHC10 | 20.95 | 0 | 1 |
| Q8WWF6_DNJB3_HUMAN | DNAJB3 | 16.55 | 0 | 1 |
| Q8WXA9_SREK1_HUMAN | SREK1 | 59.35 | 0 | 1 |
| Q8WZ60_KLHL6_HUMAN | KLHL6 | 70.31 | 0 | 1 |
| Q92552_RT27_HUMAN | MRPS27 | 47.58 | 0 | 1 |
| Q92796_DLG3_HUMAN | DLG3 | 90.26 | 0 | 1 |
| Q92820_GGH_HUMAN | GGH | 35.94 | 0 | 1 |
| Q92879_CELF1_HUMAN | CELF1 | 52.03 | 0 | 1 |
| Q96AY4_TTC28_HUMAN | TTC28 | 270.71 | 0 | 1 |
| Q96EP5_DAZP1_HUMAN | DAZAP1 | 43.36 | 0 | 1 |
| Q96EY1_DNJA3_HUMAN | DNAJA3 | 52.46 | 0 | 1 |
| Q96RY7_IF140_HUMAN | IFT140 | 165.09 | 0 | 1 |
| Q96S59_RANB9_HUMAN | RANBP9 | 77.8 | 0 | 1 |
| Q99536_VAT1_HUMAN | VAT1 | 41.89 | 0 | 1 |
| Q9BQG0_MBB1A_HUMAN | MYBBP1A | 148.76 | 0 | 1 |
| Q9BRD0_BUD13_HUMAN | BUD13 | 70.48 | 0 | 1 |
| Q9BU40_CRDL1_HUMAN | CHRDL1 | 51.13 | 0 | 1 |
| Q9BWU0_NADAP_HUMAN | SLC4A1AP | 88.76 | 0 | 1 |
| Q9BYD3_RM04_HUMAN | MRPL4 | 34.9 | 0 | 1 |
| Q9BZZ5_API5_HUMAN | API5 | 58.97 | 0 | 1 |
| Q9H116_GZF1_HUMAN | GZF1 | 80.44 | 0 | 1 |
| Q9H175_CSRN2_HUMAN | CSRNP2 | 59.55 | 0 | 1 |
| Q9H3G5_CPVL_HUMAN | CPVL | 54.13 | 0 | 1 |
| Q9H4B6_SAV1_HUMAN | SAV1 | 44.61 | 0 | 1 |
| Q9H5V9_CX056_HUMAN | CXorf56 | 25.61 | 0 | 1 |
| Q9H814_PHAX_HUMAN | PHAX | 44.38 | 0 | 1 |
| Q9H9B4_SFXN1_HUMAN | SFXN1 | 35.6 | 0 | 1 |
| Q9H9J4_UBP42_HUMAN | USP42 | 145.3 | 0 | 1 |
| Q9HB71_CYBP_HUMAN | CACYBP | 26.19 | 0 | 1 |
| Q9NPQ8_RIC8A_HUMAN | RIC8A | 59.67 | 0 | 1 |
| Q9NS69_TOM22_HUMAN | TOMM22 | 15.51 | 0 | 1 |
| Q9NWF6_Q9NWF6_HUMAN | SPATA6L | 21.91 | 0 | 1 |
| Q9P035_HACD3_HUMAN | PTPLAD1 | 43.13 | 0 | 1 |
| Q9UI42_CBPA4_HUMAN | CPA4 | 47.32 | 0 | 1 |
| Q9UJZ1_STML2_HUMAN | STOML2 | 38.51 | 0 | 1 |
| Q9UK45_LSM7_HUMAN | LSM7 | 11.6 | 0 | 1 |
| Q9UKB3_DJC12_HUMAN | DNAJC12 | 23.4 | 0 | 1 |
| Q9ULI4_KI26A_HUMAN | KIF26A | 194.47 | 0 | 1 |
| Q9Y2R5_RT17_HUMAN | MRPS17 | 14.49 | 0 | 1 |
| Q9Y3D9_RT23_HUMAN | MRPS23 | 21.76 | 0 | 1 |
| Q9Y3E5_PTH2_HUMAN | PTRH2 | 19.18 | 0 | 1 |
| Q9Y3U8_RL36_HUMAN | RPL36 | 12.25 | 0 | 1 |
| Q9Y5V3_MAGD1_HUMAN | MAGED1 | 86.11 | 0 | 1 |
| O00410_IPO5_HUMAN | IPO5 | 123.55 | 1 | 1 |
| O00571_DDX3X_HUMAN | DDX3X | 73.2 | 1 | 1 |
| O75223_GGCT_HUMAN | GGCT | 20.99 | 1 | 1 |
| P04908_H2A1B_HUMAN | HIST1H2AB | 14.13 | 1 | 1 |
| P05386_RLA1_HUMAN | RPLP1 | 11.51 | 1 | 1 |
| P06748_NPM_HUMAN | NPM1 | 32.55 | 1 | 1 |
| P14649_MYL6B_HUMAN | MYL6B | 22.75 | 1 | 1 |
| P17174_AATC_HUMAN | GOT1 | 46.22 | 1 | 1 |
| P19388_RPAB1_HUMAN | POLR2E | 24.54 | 1 | 1 |
| P25787_PSA2_HUMAN | PSMA2 | 25.88 | 1 | 1 |
| P28074_PSB5_HUMAN | PSMB5 | 28.46 | 1 | 1 |
| P30048_PRDX3_HUMAN | PRDX3 | 27.68 | 1 | 1 |
| P31483_TIA1_HUMAN | TIA1 | 42.94 | 1 | 1 |
| P46060_RAGP1_HUMAN | RANGAP1 | 63.5 | 1 | 1 |
| P49411_EFTU_HUMAN | TUFM | 49.51 | 1 | 1 |
| P49759_CLK1_HUMAN | CLK1 | 57.25 | 1 | 1 |
| P50914_RL14_HUMAN | RPL14 | 23.42 | 1 | 1 |
| P54105_ICLN_HUMAN | CLNS1A | 26.2 | 1 | 1 |
| P56537_IF6_HUMAN | EIF6 | 26.58 | 1 | 1 |
| P60953_CDC42_HUMAN | CDC42 | 21.25 | 1 | 1 |
| P61626_LYSC_HUMAN | LYZ | 16.53 | 1 | 1 |
| P62266_RS23_HUMAN | RPS23 | 15.8 | 1 | 1 |
| P62333_PRS10_HUMAN | PSMC6 | 44.15 | 1 | 1 |
| P62834_RAP1A_HUMAN | RAP1A | 20.97 | 1 | 1 |
| P62854_RS26_HUMAN | RPS26 | 13.01 | 1 | 1 |
| P62857_RS28_HUMAN | RPS28 | 7.84 | 1 | 1 |
| P62873_GBB1_HUMAN | GNB1 | 37.35 | 1 | 1 |
| P68363_TBA1B_HUMAN | TUBA1B | 50.12 | 1 | 1 |
| P78559_MAP1A_HUMAN | MAP1A | 305.3 | 1 | 1 |
| Q04837_SSBP_HUMAN | SSBP1 | 17.25 | 1 | 1 |
| B9A064_IGLL5_HUMAN | IGLL5 | 23.05 | 2 | 0 |
| E7EVS6_E7EVS6_HUMAN | ACTB | 17.87 | 1 | 0 |
| E7EX90_E7EX90_HUMAN | DCTN1 | 139.01 | 1 | 0 |
| F8W0Q9_F8W0Q9_HUMAN | PPHLN1 | 44.66 | 1 | 0 |
| F8W6A0_F8W6A0_HUMAN | PPHLN1 | 41.13 | 2 | 0 |
| H0YBL2_H0YBL2_HUMAN | SORBS3 | 16.86 | 1 | 0 |
| H0YGV5_H0YGV5_HUMAN | CYP2E1 | 4.76 | 1 | 0 |
| I3L4N8_I3L4N8_HUMAN | ACTG1 | 26.86 | 1 | 0 |
| IGHG_RABIT | | 35.38 | 1 | 0 |
| O00159_MYO1C_HUMAN | MYO1C | 121.61 | 7 | 0 |
| O00399_DCTN6_HUMAN | DCTN6 | 20.73 | 1 | 0 |
| O00451_GFRA2_HUMAN | GFRA2 | 51.51 | 1 | 0 |
| O14639_ABLM1_HUMAN | ABLIM1 | 87.63 | 7 | 0 |
| O14818_PSA7_HUMAN | PSMA7 | 27.87 | 1 | 0 |
| O15020_SPTN2_HUMAN | SPTBN2 | 271.16 | 22 | 0 |
| O15061_SYNEM_HUMAN | SYNM | 172.66 | 16 | 0 |
| O15143_ARC1B_HUMAN | ARPC1B | 40.92 | 2 | 0 |
| O15144_ARPC2_HUMAN | ARPC2 | 34.31 | 8 | 0 |
| O15294_OGT1_HUMAN | OGT | 116.85 | 6 | 0 |
| O15484_CAN5_HUMAN | CAPN5 | 73.12 | 1 | 0 |
| O15511_ARPC5_HUMAN | ARPC5 | 16.31 | 2 | 0 |
| O15514_RPB4_HUMAN | POLR2D | 16.3 | 2 | 0 |
| O43166_SI1L1_HUMAN | SIPA1L1 | 199.9 | 13 | 0 |
| O43426_SYNJ1_HUMAN | SYNJ1 | 172.99 | 1 | 0 |
| O43795_MYO1B_HUMAN | MYO1B | 131.9 | 3 | 0 |
| O60256_KPRB_HUMAN | PRPSAP2 | 40.9 | 2 | 0 |
| O60264_SMCA5_HUMAN | SMARCA5 | 121.83 | 1 | 0 |
| O60292_SI1L3_HUMAN | SIPA1L3 | 194.49 | 4 | 0 |
| O60361_NDK8_HUMAN | NME2P1 | 15.52 | 1 | 0 |
| O60784_TOM1_HUMAN | TOM1 | 53.78 | 1 | 0 |
| O75369_FLNB_HUMAN | FLNB | 277.99 | 26 | 0 |
| O75556_SG2A1_HUMAN | SCGB2A1 | 10.88 | 4 | 0 |
| O75688_PPM1B_HUMAN | PPM1B | 52.61 | 2 | 0 |
| O75955_FLOT1_HUMAN | FLOT1 | 47.33 | 1 | 0 |
| O94832_MYO1D_HUMAN | MYO1D | 116.13 | 1 | 0 |
| O94901_SUN1_HUMAN | SUN1 | 90.01 | 1 | 0 |
| O94915_FRYL_HUMAN | FRYL | 339.38 | 3 | 0 |
| O95425_SVIL_HUMAN | SVIL | 247.59 | 34 | 0 |
| O95573_ACSL3_HUMAN | ACSL3 | 80.37 | 1 | 0 |
| P00492_HPRT_HUMAN | HPRT1 | 24.56 | 2 | 0 |
| P00739_HPTR_HUMAN | HPR | 39 | 2 | 0 |
| P00751_CFAB_HUMAN | CFB | 85.48 | 1 | 0 |
| P01009_A1AT_HUMAN | SERPINA1 | 46.71 | 8 | 0 |
| P01023_A2MG_HUMAN | A2M | 163.19 | 4 | 0 |
| P01024_CO3_HUMAN | C3 | 187.03 | 10 | 0 |
| P01036_CYTS_HUMAN | CST4 | 16.2 | 1 | 0 |
| P01591_IGJ_HUMAN | IGJ | 18.09 | 10 | 0 |
| P01596_KV104_HUMAN | | 11.7 | 1 | 0 |
| P01620_KV302_HUMAN | | 11.77 | 4 | 0 |
| P01765_HV304_HUMAN | | 12.35 | 1 | 0 |
| P01766_HV305_HUMAN | | 13.22 | 1 | 0 |
| P01834_IGKC_HUMAN | IGKC | 11.6 | 5 | 0 |
| P01857_IGHG1_HUMAN | IGHG1 | 36.08 | 5 | 0 |
| P01859_IGHG2_HUMAN | IGHG2 | 35.88 | 3 | 0 |
| P01871_IGHM_HUMAN | IGHM | 49.28 | 3 | 0 |
| P01876_IGHA1_HUMAN | IGHA1 | 37.63 | 7 | 0 |
| P01877_IGHA2_HUMAN | IGHA2 | 36.5 | 1 | 0 |
| P01880_IGHD_HUMAN | IGHD | 42.23 | 1 | 0 |
| P02545_LMNA_HUMAN | LMNA | 74.09 | 2 | 0 |
| P02647_APOA1_HUMAN | APOA1 | 30.76 | 3 | 0 |
| P02675_FIBB_HUMAN | FGB | 55.89 | 2 | 0 |
| P02679_FIBG_HUMAN | FGG | 51.48 | 2 | 0 |
| P02763_A1AG1_HUMAN | ORM1 | 23.5 | 1 | 0 |
| P02766_TTHY_HUMAN | TTR | 15.88 | 2 | 0 |
| P02787_TRFE_HUMAN | TF | 77.01 | 7 | 0 |
| P02788_TRFL_HUMAN | LTF | 78.13 | 16 | 0 |
| P04004_VTNC_HUMAN | VTN | 54.27 | 1 | 0 |
| P04083_ANXA1_HUMAN | ANXA1 | 38.69 | 5 | 0 |
| P04844_RPN2_HUMAN | RPN2 | 69.24 | 1 | 0 |
| P04899_GNAI2_HUMAN | GNAI2 | 40.43 | 1 | 0 |
| P05164_PERM_HUMAN | MPO | 83.81 | 9 | 0 |
| P06312_KV401_HUMAN | IGKV4-1 | 13.37 | 1 | 0 |
| P08133_ANXA6_HUMAN | ANXA6 | 75.83 | 2 | 0 |
| P09104_ENOG_HUMAN | ENO2 | 47.24 | 1 | 0 |
| P09211_GSTP1_HUMAN | GSTP1 | 23.34 | 1 | 0 |
| P09471_GNAO_HUMAN | GNAO1 | 40.02 | 1 | 0 |
| P0C0L4_CO4A_HUMAN | C4A | 192.66 | 1 | 0 |
| P0CG05_LAC2_HUMAN | IGLC2 | 11.29 | 3 | 0 |
| P10599_THIO_HUMAN | TXN | 11.73 | 2 | 0 |
| P10909_CLUS_HUMAN | CLU | 52.46 | 4 | 0 |
| P11277_SPTB1_HUMAN | SPTB | 246.32 | 2 | 0 |
| P11908_PRPS2_HUMAN | PRPS2 | 34.75 | 1 | 0 |
| P12036_NFH_HUMAN | NEFH | 112.41 | 2 | 0 |
| P12429_ANXA3_HUMAN | ANXA3 | 36.35 | 2 | 0 |
| P13489_RINI_HUMAN | RNH1 | 49.94 | 2 | 0 |
| P13637_AT1A3_HUMAN | ATP1A3 | 111.68 | 1 | 0 |
| P13667_PDIA4_HUMAN | PDIA4 | 72.89 | 2 | 0 |
| P13796_PLSL_HUMAN | LCP1 | 70.24 | 3 | 0 |
| P13797_PLST_HUMAN | PLS3 | 70.77 | 6 | 0 |
| P14780_MMP9_HUMAN | MMP9 | 78.41 | 1 | 0 |
| P15531_NDKA_HUMAN | NME1 | 17.14 | 3 | 0 |
| P16615_AT2A2_HUMAN | ATP2A2 | 114.68 | 1 | 0 |
| P17980_PRS6A_HUMAN | PSMC3 | 49.17 | 1 | 0 |
| P18206_VINC_HUMAN | VCL | 123.72 | 2 | 0 |
| P18669_PGAM1_HUMAN | PGAM1 | 28.79 | 1 | 0 |
| P19105_ML12A_HUMAN | MYL12A | 19.78 | 5 | 0 |
| P22102_PUR2_HUMAN | GART | 107.7 | 1 | 0 |
| P27797_CALR_HUMAN | CALR | 48.11 | 2 | 0 |
| P28289_TMOD1_HUMAN | TMOD1 | 40.54 | 7 | 0 |
| P29992_GNA11_HUMAN | GNA11 | 42.1 | 1 | 0 |
| P30040_ERP29_HUMAN | ERP29 | 28.98 | 1 | 0 |
| P30101_PDIA3_HUMAN | PDIA3 | 56.75 | 1 | 0 |
| P30153_2AAA_HUMAN | PPP2R1A | 65.27 | 1 | 0 |
| P30566_PUR8_HUMAN | ADSL | 54.85 | 1 | 0 |
| P31146_COR1A_HUMAN | CORO1A | 50.99 | 1 | 0 |
| P31939_PUR9_HUMAN | ATIC | 64.58 | 1 | 0 |
| P35241_RADI_HUMAN | RDX | 68.52 | 1 | 0 |
| P35579_MYH9_HUMAN | MYH9 | 226.39 | 25 | 0 |
| P35611_ADDA_HUMAN | ADD1 | 80.9 | 2 | 0 |
| P35749_MYH11_HUMAN | MYH11 | 227.2 | 1 | 0 |
| P36873_PP1G_HUMAN | PPP1CC | 36.96 | 1 | 0 |
| P43363_MAGAA_HUMAN | MAGEA10 | 40.75 | 1 | 0 |
| P46940_IQGA1_HUMAN | IQGAP1 | 189.13 | 13 | 0 |
| P48681_NEST_HUMAN | NES | 177.33 | 1 | 0 |
| P50990_TCPQ_HUMAN | CCT8 | 59.58 | 1 | 0 |
| P51531_SMCA2_HUMAN | SMARCA2 | 181.17 | 2 | 0 |
| P51532_SMCA4_HUMAN | SMARCA4 | 184.53 | 3 | 0 |
| P51610_HCFC1_HUMAN | HCFC1 | 208.6 | 1 | 0 |
| P55786_PSA_HUMAN | NPEPPS | 103.21 | 1 | 0 |
| P56192_SYMC_HUMAN | MARS | 101.05 | 1 | 0 |
| P57678_GEMI4_HUMAN | GEMIN4 | 119.96 | 2 | 0 |
| P60228_EIF3E_HUMAN | EIF3E | 52.19 | 2 | 0 |
| P61160_ARP2_HUMAN | ACTR2 | 44.73 | 14 | 0 |
| P61981_1433G_HUMAN | YWHAG | 28.28 | 1 | 0 |
| P62158_CALM_HUMAN | CALM1 | 16.83 | 6 | 0 |
| P62487_RPB7_HUMAN | POLR2G | 19.28 | 1 | 0 |
| P62820_RAB1A_HUMAN | RAB1A | 22.66 | 1 | 0 |
| P62937_PPIA_HUMAN | PPIA | 18 | 3 | 0 |
| P63000_RAC1_HUMAN | RAC1 | 21.44 | 1 | 0 |
| P68871_HBB_HUMAN | HBB | 15.99 | 2 | 0 |
| P78371_TCPB_HUMAN | CCT2 | 57.45 | 2 | 0 |
| P80188_NGAL_HUMAN | LCN2 | 22.57 | 3 | 0 |
| P80303_NUCB2_HUMAN | NUCB2 | 50.16 | 1 | 0 |
| Q00169_PIPNA_HUMAN | PITPNA | 31.79 | 1 | 0 |
| Q01082_SPTB2_HUMAN | SPTBN1 | 274.44 | 55 | 0 |
| Q01518_CAP1_HUMAN | CAP1 | 51.87 | 1 | 0 |
| Q02880_TOP2B_HUMAN | TOP2B | 183.15 | 1 | 0 |
| Q03001_DYST_HUMAN | DST | 860.13 | 1 | 0 |
| Q03252_LMNB2_HUMAN | LMNB2 | 67.65 | 1 | 0 |
| Q03393_PTPS_HUMAN | PTS | 16.38 | 2 | 0 |
| Q08380_LG3BP_HUMAN | LGALS3BP | 65.29 | 1 | 0 |
| Q12824_SNF5_HUMAN | SMARCB1 | 44.11 | 1 | 0 |
| Q13045_FLII_HUMAN | FLII | 144.66 | 8 | 0 |
| Q13510_ASAH1_HUMAN | ASAH1 | 44.63 | 1 | 0 |
| Q13561_DCTN2_HUMAN | DCTN2 | 44.2 | 3 | 0 |
| Q13867_BLMH_HUMAN | BLMH | 52.53 | 2 | 0 |
| Q14203_DCTN1_HUMAN | DCTN1 | 141.61 | 1 | 0 |
| Q14558_KPRA_HUMAN | PRPSAP1 | 39.37 | 1 | 0 |
| Q14573_ITPR3_HUMAN | ITPR3 | 303.91 | 3 | 0 |
| Q14651_PLSI_HUMAN | PLS1 | 70.21 | 9 | 0 |
| Q15019_SEPT2_HUMAN | 2-Sep | 41.46 | 3 | 0 |
| Q15517_CDSN_HUMAN | CDSN | 51.49 | 2 | 0 |
| Q16181_SEPT7_HUMAN | 7-Sep | 50.65 | 1 | 0 |
| Q27J81_INF2_HUMAN | INF2 | 135.54 | 3 | 0 |
| Q32M84_BTBDG_HUMAN | BTBD16 | 58.44 | 1 | 0 |
| Q53QZ3_RHG15_HUMAN | ARHGAP15 | 54.51 | 1 | 0 |
| Q56A73_SPIN4_HUMAN | SPIN4 | 28.64 | 1 | 0 |
| Q5M9N0_CD158_HUMAN | CCDC158 | 127.06 | 1 | 0 |
| Q5UIP0_RIF1_HUMAN | RIF1 | 274.29 | 1 | 0 |
| Q5VUJ6_LRCH2_HUMAN | LRCH2 | 84.54 | 6 | 0 |
| Q5VWN6_F208B_HUMAN | FAM208B | 268.68 | 6 | 0 |
| Q5W0B1_RN219_HUMAN | RNF219 | 81.07 | 1 | 0 |
| Q6P4F7_RHGBA_HUMAN | ARHGAP11A | 113.8 | 4 | 0 |
| Q6PJ61_FBX46_HUMAN | FBXO46 | 64.59 | 1 | 0 |
| Q76I76_SSH2_HUMAN | SSH2 | 158.12 | 2 | 0 |
| Q7Z7M9_GALT5_HUMAN | GALNT5 | 106.2 | 1 | 0 |
| Q86WI1_PKHL1_HUMAN | PKHD1L1 | 465.44 | 1 | 0 |
| Q86X67_NUD13_HUMAN | NUDT13 | 39.66 | 1 | 0 |
| Q86XJ1_GA2L3_HUMAN | GAS2L3 | 75.17 | 2 | 0 |
| Q8IZU2_WDR17_HUMAN | WDR17 | 147.61 | 6 | 0 |
| Q8N3V7_SYNPO_HUMAN | SYNPO | 99.4 | 4 | 0 |
| Q8N556_AFAP1_HUMAN | AFAP1 | 80.67 | 2 | 0 |
| Q8NF91_SYNE1_HUMAN | SYNE1 | 1010.46 | 1 | 0 |
| Q8NFW8_NEUA_HUMAN | CMAS | 48.35 | 1 | 0 |
| Q8NHQ1_CEP70_HUMAN | CEP70 | 69.71 | 6 | 0 |
| Q8TAQ2_SMRC2_HUMAN | SMARCC2 | 132.8 | 2 | 0 |
| Q8TCU4_ALMS1_HUMAN | ALMS1 | 460.68 | 6 | 0 |
| Q8TDL5_BPIB1_HUMAN | BPIFB1 | 52.41 | 5 | 0 |
| Q8WU79_SMAP2_HUMAN | SMAP2 | 46.75 | 1 | 0 |
| Q8WWI1_LMO7_HUMAN | LMO7 | 192.58 | 4 | 0 |
| Q8WXF1_PSPC1_HUMAN | PSPC1 | 58.71 | 2 | 0 |
| Q8WYL5_SSH1_HUMAN | SSH1 | 115.44 | 1 | 0 |
| Q8WZ42_TITIN_HUMAN | TTN | 3813.65 | 1 | 0 |
| Q92547_TOPB1_HUMAN | TOPBP1 | 170.57 | 1 | 0 |
| Q92747_ARC1A_HUMAN | ARPC1A | 41.54 | 3 | 0 |
| Q92785_REQU_HUMAN | DPF2 | 44.13 | 1 | 0 |
| Q92828_COR2A_HUMAN | CORO2A | 59.73 | 2 | 0 |
| Q96C19_EFHD2_HUMAN | EFHD2 | 26.68 | 4 | 0 |
| Q96DA0_ZG16B_HUMAN | ZG16B | 22.72 | 2 | 0 |
| Q96FF7_YS003_HUMAN | | 24.01 | 1 | 0 |
| Q96GM5_SMRD1_HUMAN | SMARCD1 | 58.2 | 1 | 0 |
| Q96HP0_DOCK6_HUMAN | DOCK6 | 229.41 | 3 | 0 |
| Q96N67_DOCK7_HUMAN | DOCK7 | 242.41 | 37 | 0 |
| Q96SB3_NEB2_HUMAN | PPP1R9B | 89.14 | 22 | 0 |
| Q9BPX5_ARP5L_HUMAN | ARPC5L | 16.93 | 2 | 0 |
| Q9BQI0_AIF1L_HUMAN | AIF1L | 17.06 | 4 | 0 |
| Q9BR76_COR1B_HUMAN | CORO1B | 54.2 | 1 | 0 |
| Q9BTE1_DCTN5_HUMAN | DCTN5 | 20.11 | 1 | 0 |
| Q9BUP0_EFHD1_HUMAN | EFHD1 | 26.91 | 15 | 0 |
| Q9BVI4_NOC4L_HUMAN | NOC4L | 58.43 | 1 | 0 |
| Q9C0G6_DYH6_HUMAN | DNAH6 | 475.68 | 2 | 0 |
| Q9GZZ8_LACRT_HUMAN | LACRT | 14.24 | 2 | 0 |
| Q9H160_ING2_HUMAN | ING2 | 32.79 | 3 | 0 |
| Q9H1A7_RPB1C_HUMAN | POLR2J3 | 13.08 | 1 | 0 |
| Q9HAR2_LPHN3_HUMAN | LPHN3 | 161.71 | 1 | 0 |
| Q9HAZ1_CLK4_HUMAN | CLK4 | 57.46 | 3 | 0 |
| Q9HC77_CENPJ_HUMAN | CENPJ | 152.91 | 1 | 0 |
| Q9NP55_BPIA1_HUMAN | BPIFA1 | 26.7 | 3 | 0 |
| Q9NR28_DBLOH_HUMAN | DIABLO | 27.11 | 1 | 0 |
| Q9NVR2_INT10_HUMAN | INTS10 | 82.18 | 1 | 0 |
| Q9NZ32_ARP10_HUMAN | ACTR10 | 46.28 | 3 | 0 |
| Q9P1U1_ARP3B_HUMAN | ACTR3B | 47.58 | 4 | 0 |
| Q9P1Y6_PHRF1_HUMAN | PHRF1 | 178.56 | 8 | 0 |
| Q9P2M7_CING_HUMAN | CGN | 136.3 | 3 | 0 |
| Q9UBC5_MYO1A_HUMAN | MYO1A | 118.33 | 2 | 0 |
| Q9UHD8_SEPT9_HUMAN | 9-Sep | 65.36 | 1 | 0 |
| Q9UJC5_SH3L2_HUMAN | SH3BGRL2 | 12.32 | 2 | 0 |
| Q9UJW0_DCTN4_HUMAN | DCTN4 | 52.3 | 6 | 0 |
| Q9ULJ8_NEB1_HUMAN | PPP1R9A | 123.27 | 17 | 0 |
| Q9ULV4_COR1C_HUMAN | CORO1C | 53.22 | 2 | 0 |
| Q9UM54_MYO6_HUMAN | MYO6 | 149.6 | 2 | 0 |
| Q9Y230_RUVB2_HUMAN | RUVBL2 | 51.12 | 3 | 0 |
| Q9Y2H1_ST38L_HUMAN | STK38L | 53.97 | 4 | 0 |
| Q9Y2L9_LRCH1_HUMAN | LRCH1 | 80.82 | 3 | 0 |
| Q9Y490_TLN1_HUMAN | TLN1 | 269.6 | 1 | 0 |
| Q9Y4I1_MYO5A_HUMAN | MYO5A | 215.27 | 15 | 0 |
| Q9Y608_LRRF2_HUMAN | LRRFIP2 | 82.12 | 2 | 0 |
| X1WI33_X1WI33_HUMAN | DYDC1 | 18.03 | 2 | 0 |

1. A method for treating cancer in a subject, comprising:
(1) identifying in the subject the presence of a mutation in a splicing factor selected from the group consisting of U2AF1, SF3B1, SRSF2, and ZRSR2; and/or determining in the subject an increased amount of DCAF15 compared to a control; and
(2) inhibiting an activity of RBM39 in the subject.

2. The method of claim 1, wherein the inhibiting step comprises promoting RBM39 degradation, preferably in a DCAF15-dependent manner.

3. The method of claim 2, wherein the promoting step comprises administering an effective amount of a compound that targets RBM39, preferably an aryl sulfonamide selected from the group consisting of indisulam, tasisulam, chloroquinoxaline sulfonamide, and analogues of each of the foregoing.

4. A method for determining whether or not a cancer patient is likely to respond to treatment, comprising the step of determining whether the patient's cancer cells have (1) a mutation in a splicing factor selected from the group consisting of U2AF1, SF3B1, SRSF2, and ZRSR2; and/or (2) an increased amount of DCAF1.5 compared to a control, wherein the mutation or increased amount indicates that the patient is likely to respond to treatment with a compound that targets RBM39, preferably an aryl sulfonamide selected from the group consisting of indisulam, tasisulam, chloroquinoxaline sulfonamide, and analogues of each of the foregoing.

5. A method for selectively treating a patient with cancer, comprising:
a. identifying a patient having (1) a mutation in a splicing factor selected from the group consisting of U2AF1, SF3B1, SRSF2, and ZRSR2; and/or (2) an increased amount of DCAF15 in the patient's cancer cells compared to a control; and
b. administering to the patient a therapeutically effective amount of a compound that targets RBM39, preferably an aryl sulfonamide selected from the group consisting of indisulam, tasisulam, chloroquinoxaline sulfonamide, and analogues of each of the foregoing.

6. The method of claim 1, wherein the mutation is a point mutation, deletion or insertion, wherein preferably the mutation is detected by sequencing.

7. The method of claim 1, wherein the increased amount of DCAF15 is an increase in gene copy number and/or nucleic acid expression and is determined using one or more of real-time (RT)-PCR, RNA sequencing (RNA-seq), microarray analysis, serial analysis of gene expression (SAGE), MassARRAY® technique, immunohistochemistry and fluorescence in situ hybridization (FISH).

8. The method of claim 1, wherein the control is from a non-cancerous sample of the patient.

9. The method of claim 1, wherein the cancer is carcinoma, lymphoma, blastoma (including medulloblastoma and retinoblastoma), sarcoma (including liposarcoma and synovial cell sarcoma), neuroendocrine tumors (including carcinoid tumors, gastrinoma, and islet cell cancer), mesothelioma, schwannoma (including acoustic neuroma), meningioma, adenocarcinoma, melanoma, and leukemia or lymphoid malignancies. More particular examples of such cancers include squamous cell cancer (e.g., epithelial squamous cell cancer), lung cancer including small-cell lung cancer (SCLC), non-small cell lung cancer (NSCLC), adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer (including metastatic breast cancer), colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma; penile carcinoma, testicular cancer, esophageal cancer, tumors of the biliary tract, as well as head and neck cancer. In some embodiments, the cancer is triple-negative metastatic breast cancer, including any histologically confirmed triple-negative (ER-, PR-, HER2-) adenocarcinoma of the breast with locally recurrent or metastatic disease (where the locally recurrent disease is not amenable to resection with curative intent).

10. The method of claim 1, wherein the cancer is leukemia or lymphoma.

11. A diagnostic kit comprising one or more reagent for determining (1) a mutation in a splicing factor selected from the group consisting of U2AF1, SF3B1, SRSF2, and ZRSR2; and/or (2) a level of DCAF15 in a sample from a cancer patient, wherein the presence of the mutation and/or an increased amount of DCAF15 compared to a control indicates responsiveness to treatment with a compound that targets RBM39, preferably an aryl sulfonamide selected from the group consisting of indisulam, tasisulam, chloroquinoxaline sulfonamide, and analogues of each of the foregoing.

12. The kit of claim 11, wherein the mutation is a point mutation, deletion or insertion, wherein preferably the mutation is detected by sequencing.

13. The kit of claim 11, wherein the increased amount of DCAF15 is an increase in gene copy number and/or nucleic acid expression and is determined using one or more of RT-PCR, RNA-seq, microarray analysis, SAGE, MassARRAY® technique, immunohistochemistry and FISH.

14. The kit of claim 11, wherein the control is from a non-cancerous sample of the patient.

15. The kit of claim 11, wherein the cancer is carcinoma, lymphoma, blastoma (including medulloblastoma and retinoblastoma), sarcoma (including liposarcoma and synovial cell sarcoma), neuroendocrine tumors (including carcinoid tumors, gastrinoma, and islet cell cancer), mesothelioma, schwannoma (including acoustic neuroma), meningioma, adenocarcinoma, melanoma, and leukemia or lymphoid malignancies. More particular examples of such cancers include squamous cell cancer (e.g., epithelial squamous cell cancer), lung cancer including small-cell lung cancer (SCLC), non-small cell lung cancer (NSCLC), adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer (including metastatic breast cancer), colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, testicular cancer, esophageal cancer, tumors of the biliary tract, as well as head and neck cancer. In some embodiments, the cancer is triple-negative metastatic breast cancer, including any histologically confirmed triple-negative (ER-, PR-, HER2-) adenocarcinoma of the breast with locally recurrent or metastatic disease (where the locally recurrent disease is not amenable to resection with curative intent).

16. The kit of claim 11, wherein the cancer is leukemia or lymphoma.
